(19) United States
(12) Patent Application Publication
(10) Pub. No.: US 2005/0276785 A1
(43) Pub. Date: Dec. 15, 2005

(54) Treatment of cardiomyopathy and of endothelial dysfunction

(75) Inventors: Georg Groetzbach, Niemgk (DE); Joachim-Friedrich Kapp, Berlin (DE); Uwe Kuehl, Berlin (DE); Heinz-Peter Schultheiss, Berlin (DE); Olaf Sowade, Berlin (DE); Claus-Steffen Stuerzebecher, Berlin (DE)

Correspondence Address: BERLEX BIOSCIENCES;PATENT DEPARTMENT, 2600 HILLTOP DRIVE, P.O. BOX 4099, RICHMOND, CA, 94804-0099, US

(73) Assignee: Schering Aktiengesellschaft, Berlin (DE)

(21) Appl. No.: 11/147,492

(22) Filed: Jun. 7, 2005

Related U.S. Application Data
(60) Provisional application No. 60/579,024, filed on Jun. 9, 2004.

(51) Int. Cl. A61K 38/21

(52) U.S. Cl. 424/85.6

(57) ABSTRACT

The present invention relates to pharmaceutical compositions comprising a therapeutically effective dose of an isolated interferon (IFN) or IFN mutein for treatment of cardiomyopathy and of endothelial dysfunction, and to methods of treating cardiomyopathy and methods of treating endothelial dysfunction using such pharmaceutical compositions. Particularly, the pharmaceutical compositions of the present invention comprise a therapeutically effective dose of an isolated, human IFN, or IFN mutein that is a variant of an isolated human IFN, e.g., an isolated, native, human IFNβ or an isolated, native, human IFNα.

This application claims priority of U.S. Provisional Application Ser. No. 60/579,024, filed 9 Jun. 2004, which is incorporated by reference herein in its entirety.

### FIELD OF THE INVENTION

The present invention is directed to pharmaceutical compositions comprising a therapeutically effective dose of an isolated interferon (IFN) or IFN mutein for treatment of cardiomyopathy and for treatment of endothelial dysfunction, and to methods of treating cardiomyopathy and methods of treating endothelial dysfunction using such pharmaceutical compositions. Particularly, the pharmaceutical compositions of the present invention comprise a therapeutically effective dose of an isolated, human IFN, or IFN mutein that is a variant of an isolated human IFN, e.g., an isolated, native, human IFNβ or an isolated, native, human IFNα.

### BACKGROUND OF THE INVENTION

Chronic inflammatory cardiomyopathy is a focal or diffuse, chronic recurrent inflammatory process in the myocardium which may be induced, e.g., by physical, chemical, and/or infectious agents. Infections, such as acute rheumatic fever and viral infections, may cause a number of types of cardiomyopathies. However, some cardiomyopathies are apparently not related to an infectious process. Moreover, cardiomyopathy can result from a variety of structural or functional abnormalities of the ventricular myocardium. Some cardiomyopathies are congenital and may cause enlargement of the heart. Metabolic diseases associated with endocrine disorders may also cause cardiomyopathies. Cardiomyopathy may also occur as a manifestation of a generalized hypersensitivity, allergic or immunologic reaction. Also, cardiomyopathy may occur as a result of collagen diseases (see e.g., Circulation (1996) 93: 841-842).

In many patients suffering from cardiomyopathy, Enteroviruses, especially group B Coxsackie-viruses, and Adenoviruses are the pathologic agents (see e.g., Kandolf, R. (1998) Med. Klin. 93: 215-222; Woodruff et al. (1980) Am. J. Pathol. 101: 425-484; Stille-Siegener et al. (1993) Idiopathic Dilated Cardiomyopathy. Springer-Verlag Berlin Heidelberg, Figulla et al. (Eds.), pp 369-372; Heim et al. (1994) Clin. Cardiol. 17: 563-565). Moreover, Adenovirus, Enterovirus, Herpesvirus, and Parvovirus have been reported in endomyocardial biopsies of patients with cardiomyopathy (see e.g., Kandolf et al. (1993) Idiopathic Dilated Cardiomyopathy. Springer-Verlag Berlin Heidelberg, Figulla et al. (Eds.), pp. 267-283; Pauschinger et al. (1999) Circulation 99:889-895; Pauschinger et al. (1999) Circulation 99:1348-1354). However, clinically, cardiomyopathy may appear in a wide variety of forms, ranging from a total lack of clinical manifestations to progressive heart failure and sudden death.

Examples of clinical manifestations of cardiomyopathy include, but are not limited to, hypertrophic cardiomyopathy, restrictive cardiomyopathy, and dilated cardiomyopathy. Dilated cardiomyopathy is a disorder of myocardial function where ventricular dilation occurs, and is known to follow viral infection. Restrictive cardiomyopathy may occur as a consequence of the ventricular walls becoming rigid so that the chambers are unable to fill adequately and can be idiopathic. Hypertrophic cardiomyopathy can be characterized by ventricular hypertrophy and may be congenital or acquired.

Currently, patients with cardiomyopathy having chronic heart failure are treated symptomatically and/or surgically for heart failure. Known treatments of cardiomyopathy involve restricted activity, stress avoidance, treatment with β-blockers, prophylactic antibiotic therapy, use of anti-coagulants, calcium channel blockers, surgery, and cardiac transplantation, but these treatments have limited efficacy and, in the case of surgery and cardiac transplantation, rely on major and potentially debilitating invasive procedures. In addition, some reports have focused on the use of somatotropin, a growth hormone, for the treatment of cardiomyopathy (see e.g., N.E. J. of Med. (1996) 334(13): 811-814). However, there is some likelihood, of inducing cancer, arrhythmias, and other problems with growth hormone therapy using the doses used in such studies.

The standard known therapy for treatment of e.g., dilated cardiomyopathy consists of e.g. ACE inhibitor, diuretic, digitalis, β-blocker, aldosterone antagonist, anticoagulant, and/or antiarrhythmic drug. However, optimal treatment with ACE inhibitors, glycosides, β-blockers, and diuretics does not directly influence specific underlying pathomechanisms of chronic heart disease and therefore may delay but not prevent progression of the disease. Moreover, such therapy is not specific and, consequently, virus-positive cardiomyopathy patients may benefit from specific antiviral strategies.

There are some reports that suggest that interferons may have an effect on chronic inflammatory cardiomyopathy (without Adenovirus, Enterovirus, Parvovirus, Human Herpes Virus 6). However, in most of these studies (see e.g., Miric et al. (1995) Eur. Heart J. 16(0): 150-152;), Miric et al. (1996) Heart 75: 596-601) there was no distinction between inflammatory cardiomyopathy caused by viruses or by other agents. The increase of natural killer cell activity and the concurrent decrease in neutralizing antibody viral titers in the IFNβ-treated cohort was interpreted as induction of antiviral immune activation (see e.g., Miric et al. (1996) Heart 75: 596-601). However, no proof of virus elimination was presented, because viral genomes were not documented before and after treatment. Thus, an effective treatment for cardiomyopathy using interferons has not been demonstrated.

Endothelium dependent flow-mediated vasodilation of systemic arteries was found to be impaired in patients with non-ischemic cardiomyopathy and virus persistence (see Vallbracht et al. (2004) Circulation 110: 2938-2945). In some cases and to different degrees this may be associated with inflammatory signs and is also seen in the coronary microcirculation ( see Vallbracht et al. (2002) JACC 40.3: 510-520, Vallbracht et al. (2004) Circulation 111: 1784-1791). The finding of impaired endothelial function in systemic or coronary blood vessels in patients with cardiomyopathy is apparently independent of the type of virus found in the endomyocardial specimen of these patients with cardiomyopathy. An apparent correlation of the degree of endothelial dysfunction to inflammatory markers found systemically or in the cardiac tissue would contribute to assuming a major role of secondary inflammatory mechanisms that lead to the manifestation of endothelial dysfunction and ist sequealae. Such mechanisms can be triggered by a virus, but also other pathogeneic mechanisms independent of virus.

Parvovirus, as one example, directly affects endothelium of arteries, arterial and venous side of capilaries and venules. In many cases this finding of virus presence in this tissue is not associated with disease. However, if symptoms occur, the resulting vascular and endothelial functional impairement may be related to immunological defense triggered by neo-antigen formation and leading to autoimmune disease. There may be infection, or presence of viral genome, without direct cytopathogenic effect. In several described cases there was parvovirus associated endothelial affection connected with secondary skin or organ damage assuming a secondary inflammatory damage induced by the virus.

Parvovirus mediated vascular pathology—in absence of a direct cytopathogenic effect—was also associated with increased e-selectin. This pro-inflammatory adhesion molecule e-selectin is one of the markers associated specifically with arterial hypertension but also found in association with atherosclerosis.

Therapeutic agents in artherosclerosis and hypertension target also endothelial dysfunction. For example, long established therapy with ACE inhibitors reduce elevated serum markers in hypertensive patients but do not affect their level in normal controls. This may be mediated by reducing damaging factors to the endothelium by the Renin-angiotensin-bradykinin system.

Organ damage may occur disjunct to measurable IgM serum antibodies but IgG antibodies are usually found. However, a majority of adults probably has IgG antibodies to Parvovirus B19 but no direct disease correlate. Organ involvement of a Parvovirus infection is not predictable and the epidmiologically seen pattern of infection leading to organ damage in adults that are non-immunocompromised cannot be well correlated with common markers. Genetic factors may play a role. The association of Parvovirus infection with chronic heart disease mediated by endothelia cell affection has been assumed.

Parvovirus diagnostics play a broad role in blood product screening but is otherwise uncommon. For example donors for blood products in whom parvoviral genome can be found to a certain degree are normally found healthy. An association with hypertension or atherosclerosis has not been reported.

There is no known direct data that would permit assessing whether chronic endothelial damage by parvovirus is a pathogeneic mechanism inducing a subset of or contributing to arterial hypertension or emerging artheroscierosis, particularly in non-diabetics. Neither has been reported that antiviral therapy would affect these conditions, an association of improvements of endothelial function under IFNβ therapy in human has never been considered.

Interferons are a family of small immunomodulatory proteins. Three types of interferons have been identified: IFNα, derived from leukocytes, IFNβ derived from fibroblasts, and IFNγ derived from lymphocytes. The important role played by IFNs as a natural defense against inflammation is documented by the observation that the inhibition of IFN production or action enhances the severity of infection.

Interferons react with specific receptors on cell surfaces to activate cytoplasmic signal proteins. These proteins enter the nucleus to stimulate cellular genes encoding a number of other proteins responsible for the defensive activity, including antiviral, antiproliferative, antitumor, and immunomodulatory effects. The important role played by the IFNs as a natural defense against viruses is documented by three types of experimental and clinical observations: in many viral infections, a strong correlation has been established between IFN production and natural recovery; inhibition of IFN production or action enhances the severity of infection, and treatment with IFN protects against viral infection.

Immunomodulatory effects of interferons include activation of macrophages and natural killer cells as well as enhancement of major histocompatibility complex (MHC) antigen expression (Baron et al. 1992). One hypothesis as to how IFNβ affects inflammation focuses on the immunomodulatory effect of the compound and is based on the findings that inflammation in the myocardium with a T-cell infiltrate biased towards a Th2 subtype is associated with a reduced spontaneous healing of myocarditis. IFNγ can, at low doses, act synergistically with (low concentrations) of IFNγ while being an antagonist of IFNγ at higher doses, e.g. with regard to the expression of MHC II. There is an initial increase of IFNγ (also) observed in MS patients when IFNβ therapy is started. IFNβ may modulate the immune system in a baseline-dependent fashion, i.e. redirect a Th1 biased immune system towards a Th2 pattern whereas a Th2 biased baseline could be redirected to a Th1 pattern. This dual mechanism may further depend on the underlying disease pathology, i.e., virus infection in chronic viral cardiomyopathy (CVC) or endothelial dysfunction connected to cardiomyopathy or other disease versus autoimmune attack e.g., in multiple sclerosis(MS).

Antiviral effects of IFN in an in vitro model of persistent enteroviral infection and its efficacy in preventing viral myocarditis in susceptible rodents have been reported (see e.g., Heim et al. (1992) J. Infect. Dis. 166: 958-965; Matsumori et al. (1988) Am. Heart J. 115: 1229-1232; Yamamoto et al. (1998) Circulation 97: 1017-1023; Wessely et al. (2001) Circulation 103: 756-761; Horwitz et al. (2000) Nature Med. 6: 693-697). Moreover, interferons, e.g., IFNα, IFNβ, and IFNγ, have been tested in in vitro assays with cultured myocardial fibroblasts. Cells were exposed to Coxsackie B3 viruses and after 7 days, when a persistent infection had developed, interferon was added. Recombinant IFNβ and IFNγ were both active and reduced virus yields. Recombinant IFNα-2a was at least 120 times less active than IFNβ (Heim et al. (1996) J Interferon Cytokine Res 16: 283-287). Apart from the direct virostatic effect of IFNβ, immunomodulatory effects are induced that may suppress virus spreading and facilitate virus clearance (see e.g., Ramshaw et al. (1997) Immunol. Rev. 159: 119-135). However, thus far no effective treatment for viral cardiomyopathy or endothelial dysfunction e.g., endothelial dysfunction connected to cardiomyopathy, using interferon has been demonstrated.

Accordingly, there remains a need for a safe and therapeutically effective treatment for cardiomyopathy and for endothelial dysfunction e.g., endothelial dysfunction connected to cardiomyopathy, that does not suffer from the limitations of the prior methods.

### SUMMARY OF THE INVENTION

The present invention provides pharmaceutical compositions comprising a therapeutically effective dose of an isolated interferon (IFN) or IFN mutein for treatment of cardiomyopathy and for treatment of endothelial dysfunction e.g., endothelial dysfunction connected to cardiomyopathy, and methods of treating cardiomyopathy and methods of treating endothelial dysfunction e.g., endothelial dysfunction connected to cardiomyopathy, using such pharmaceutical compositions. Preferably, the isolated IFN is an isolated, human IFN and the IFN mutein is a variant of an isolated, human IFN. Examples of an isolated, human IFN of the present invention include, but are not limited to IFNα, IFNβ (e.g., IFNβ-1a), and IFNγ. An example of an IFN mutein, include, but is not limited to, an IFNβ mutein, e.g., IFNβ-1 b (also termed IFNβₛₑᵣ₁₇-1b, Betaseron®, Betaferon®). In a preferred aspect, the isolated IFN is an isolated, native, human IFNβ or an isolated, native human IFNα. In another preferred aspect, the IFN mutein is a variant of an isolated, native, human IFNβ or a variant of an isolated, native human IFNα.

In some aspects, the therapeutically effective amount of IFN or IFN mutein of the present invention is in a range that is from about 30 mcg to at least about 1000 mcg.

In some aspects, the therapeutically effective amount of the IFN or IFN mutein of the present invention is in a range from about 30 mcg to about 500 mcg. For example, in some aspects the therapeutically effective amount is about 30 mcg to about 50 mcg, about 50 mcg to about 75 mcg, about 75 mcg to about 100 mcg, or about 100 mcg to about 125 mcg, about 125 mcg to about 150 mcg, about 150 mcg to about 175 mcg, about 175 mcg to about 200 mcg, about 200 mcg to about 225 mcg, about 225 mcg to about 250 mcg, about 250 mcg to about 275 mcg, about 275 mcg to about 300 mcg, about 300 mcg to about 325 mcg, about 325 mcg to about 350 mcg, about 350 mcg to about 375 mcg, about 375 mcg to about 400 mcg, about 400 mcg to about 425 mcg, about 425 mcg to about 450 mcg, about 450 mcg to about 475 mcg, or about 475 mcg to about 500 mcg. In a preferred aspect, the therapeutically effective amount is about 30 mcg. In another preferred aspect, the therapeutically effective amount is about 250 mcg. In another preferred aspect, the therapeutically effective amount is about 500 mcg.

In some aspects, the therapeutically effective amount of the IFN or IFN mutein of the present invention is in a range that is at least about 500 mcg to at least about 1000 mcg. For example, in some aspects, the therapeutically effective amount of IFN or IFN mutein is at least about 500 mcg to at least about 525 mcg, at least about 525 mcg to at least about 550 mcg, at least about 550 mcg to at least about 575 mcg, at least about 575 mcg to about at least about 600 mcg, at least about 600 mcg to at least about 625 mcg, at least about 625 mcg to at least about 650 mcg, at least about 650 mcg to at least about 675 mcg, at least about 675 mcg to at least about 700 mcg, at least about 700 mcg to at least about 725 mcg, at least about 725 mcg to at least about 750 mcg, at least about 750 mcg to at least about 775 mcg, at least about 800 mcg to at least about 825 mcg, at least about 825 mcg to at least about 850 mcg, at least about 850 mcg to at least about 875 mcg, at least about 875 mcg to at least about 900 mcg, at least about 900 mcg to at least about 925 mcg, at least about 925 mcg to at least about 950 mcg, at least about 950 mcg to at least about 975 mcg, or at least about 975 mcg to at least about 1000 mcg.

The isolated IFN or IFN mutein of the present invention is preferably a synthetic or recombinant polypeptide. In one aspect, the isolated IFN of the present invention is an isolated, native, human IFNβ or an isolated, native, human IFNα. In another aspect, the IFN mutein of the present invention is a variant of a biologically active, native, human IFN, e.g., a native, human IFNβ or native, human IFNα. In a preferred aspect, the IFN mutein of the present invention is IFNβ-1b.

In one aspect, the pharmaceutical compositions of the present invention are stabilized, human serum albumin-free (HSA-free) pharmaceutical compositions. Preferably, the stabilized, HSA-free pharmaceutical compositions of the present invention comprise an IFN or IFN mutein that is substantially monomeric and solubilized in a low-ionic-strength formulation. In a preferred aspect, the IFN is a native, human IFNβ or a native, human IFNα, and the IFN mutein is a variant of a native, human IFNβ or a variant of a native human IFNα.

In one aspect, the isolated IFN or IFN mutein of the present invention is PEGylated. In a preferred aspect, the PEGylated IFN is a PEGylated native, human IFNβ or a PEGylated native, human IFNα, and the PEGylated IFN mutein is a variant of a native, human IFNβ or a variant of a native human IFα.

In one aspect, the IFN mutein of the present invention (e.g., IFNβ mutein) is a variant of a biologically active, native IFNβ, where: 1) the native IFNβ has at least one cysteine residue that is free to form a disulfide link and is nonessential to the biological activity of the native IFNβ; 2) the amino acid positions of the IFN mutein are numbered in accordance with the native IFNβ; and 3) the IFN mutein has at least one cysteine residue deleted or replaced by another amino acid residue, and exhibits the biological activity of native IFNβ. Preferably, the native IFNβ is a native, human IFNβ.

In another aspect, the isolated IFN or IFN mutein of the present invention have other modifications. For example, the IFN or IFN mutein of the present invention can lack an N-terminal methionine, may or may not be glycosylated, and/or may have a secretion signal sequence or other additional sequences e.g., fused thereto.

In one asepct, the invention provides a pharmaceutical composition having IFNβ activity and comprising a therapeutically effective amount of an isolated IFNβ or IFNβ mutein, wherein the therapeutically effective amount is in a range from about 30 mcg to about 500 mcg. In one aspect, the therapeutically effective amount is about 30 mcg, about 250 mcg, or about 500 mcg.

In one asepct, the invention provides a pharmaceutical composition having IFNβ activity and comprising a therapeutically effective amount of an isolated IFNα or IFNα mutein, wherein the therapeutically effective amount is in a range from about 30 mcg to about 500 mcg. In one aspect, the therapeutically effective amount is about 30 mcg, about 250 mcg, or about 500 mcg.

In another aspect, the IFNβ mutein of the present invention has a cysteine at position 17 that is replaced by a neutral amino acid selected from a group consisting of serine, threonine, glycine, alanine, valine, leucine, isoleucine, histidine, tyrosine, phenylalanine, tryptophan, and methionine. In one aspect, the neutral amino acid is serine.

In another aspect, the isolated IFN or IFN mutein of the present invention lacks an N-terminal methionine. In a preferred aspect, the IFN is a native, human IFNβ and the IFN mutein is a variant of a native, human IFNβ. In another preferred aspect, the IFN is a native, human IFNα and the IFN mutein is a variant of a native, human IFNα.

In one aspect, the invention provides a pharmaceutical composition having IFNβ activity and comprising a therapeutically effective amount of an isolated IFNβ or IFNβ mutein, wherein the therapeutically effective amount is about 500 mcg, and wherein the isolated IFNβ mutein is a variant of a human IFNβ where a cysteine at position 17 is replaced by a serine.

In another aspect, the isolated IFNβ mutein of the present invention is Betaseron®.

In one aspect, the present invention provides a stabilized, HSA-free pharmaceutical composition comprising an IFNβ (e.g., IFNβ-1a) or IFNβ mutein (e.g., IFNβ-1b). In another aspect, the stabilized, HSA-free pharmaceutical composition of the present invention comprises an IFNβ (e.g., IFNβ-1a) or IFNβ mutein (e.g., IFNβ-1b) that is substantially monomeric and solubilized in a low-ionic-strength formulation. In a related aspect, the low-ionic-strength formulation is a solution having a pH from about 2 to about 5, and an ionic strength from about 1 mM to about 100 mM.

In one aspect, the present invention provides a stabilized, HSA-free pharmaceutical composition comprising an IFNα or IFNα mutein. In another aspect, the stabilized, HSA-free pharmaceutical composition of the present invention comprises an IFNα or IFNα mutein that is substantially monomeric and solubilized in a low-ionic-strength formulation. In a related aspect, the low-ionic-strength formulation is a solution having a pH from about 2 to about 5, and an ionic strength from about 1 mM to about 100 mM.

In one aspect, the present invention provides a method of treating a patient for cardiomyopathy and/or endothelial dysfunction e.g., endothelial dysfunction connected to cardiomyopathy, comprising administering to the patient a pharmaceutical composition of the present invention (as described herein).

### BRIEF DESCRIPTION OF THE DRAWINGS

The foregoing and other objects of the present invention, the various features thereof, as well as the invention itself may be more fully understood from the following description.

FIG. 1 is a schematic illustrating the therapeutic effects of murine IFNα-2 (“IFNα-2”) and murine IFNβ (“IFNβ”) on the reduction of coxsackievirus B3-induced weight loss in Balb/c mice. The percent change of body weight is calculated from pre-treatment values. The time course is illustrated in FIG. 1A, and the corresponding dose-response curve of the change in body weight on day 7 is illustrated in FIG. 1B.

Dose (MIU/kg) murine IFNβ (¢IFNb”) and murine IFNα-2 (“IFNa”): murine IFNβ<0.01 MIU/kg (“IFNb<0.01”); murine IFNβ 2.5 MIU/kg (“IFNb2.5”); murine IFNβ 5 MIU/kg (“IFNb5”); murine IFNβ10 MIU/kg (“IFNb10”); and murine IFNα-2 10 MIU/kg (“IFNa10”).

FIG. 2 are photos illustrating histopathology cross-sections for the heart from naive or coxsackievirus B3-infected Balb/c mice treated with or without 10 MIU/kg murine IFNα-2 (“IFN α”) and murine IFNβ (“IFN β”).

FIG. 3 is a schematic illustrating that murine IFNα-2 (“Interferon a-2”) and murine IFNβ (“Interferon b”) prevented coxsackievirus B-3-induced death in Balb/c mice.

FIGS. 4A and 4B are schematics illustrating that murine IFNβ (“IFNb” or “IFN β”) and murine IFNα-2 (“IFNa” or “IFN α-2”) dose-dependently attenuated coxsackievirus B3-induced cardiac thrombosis (% incidence) in Balb/c mice. The time course is illustrated in FIG. 4A, and the corresponding dose-response curve of the average incidence of thrombosis on day 4 and 7 is illustrated in FIG. 4B.

Dose (MIU/kg) murine IFNβ (“IFNb”) and murine IFNα-2 (“IFNa”): murine IFNβ<0.01 MIU/kg (“IFNb<0.01”); murine IFNβ 2.5 MIU/kg (“IFNb2.5”); murine IFNβ 5 MIU/kg (“IFNb5”); murine IFNβ 10 MIU/kg (“IFNb10”); and murine IFNα-2 10 MIU/kg (“IFNa10”).

FIGS. 5A to 5H are schematics illustrating that murine IFNβ (“IFNb” or “IFN β”) and murine IFNα-2 (“IFNa” or “IFN α-2”) dose-dependently attenuated coxsackievirus B3-induced myocarditis semi-quantified by 3 different pathology scores (inflammatory infiltration, myocyte injury and fluorescence) in Balb/c mice. An average score was used to measure the overall severity of myocarditis. The time course is illustrated in FIGS. 5A, C, E, and G and the corresponding dose-response curve on day 7 is illustrated in FIGS. 5B, D, F, and H, respectively.

Dose (MIU/kg) murine IFNβ (“IFNb”) and murine IFNα-2 (“IFNa”): murine IFNβ<0.01 MIU/kg (“IFNb<0.01”); murine IFNβ 2.5 MIU/kg (“IFNb2.5”); murine IFNβ 5 MIU/kg (“IFNb5”); murine IFNβ 10. MIU/kg (“IFNb10”); and murine IFNα-2 10 MIU/kg (“IFNa10”).

FIGS. 6A to 6D are schematics illustrating that murine IFNβ (“IFNb” or “IFN β”) and murine IFNα-2 (“IFNa” or “IFN α-2”) dose-dependently reduced cardiac viral load determined by plaque-forming assay (FIGS. 6A and 6B), and CVB3 mRNA (FIGS. 6C and 6D) in Balb/c mice inoculated with coxsackievirus B3. The time course is illustrated in FIGS. 6A and 6C, and the corresponding dose-response curve at day 4 is illustrated in FIG. 6B and 6D, respectively.

Dose (MIU/kg) murine IFNβ (“IFNb”) and murine IFNα-2 (“IFNa”): murine IFNβ<0.01 MIU/kg (“IFNb<0.01”); murine IFNβ 2.5 MIU/kg (“IFNb2.5”); murine IFNβ 5 MIU/kg (“IFNb5”); murine IFNβ 10 MIU/kg (“IFNb10”); and murine IFNα-2 10 MIU/kg (“IFNa10”).

### DETAILED DESCRIPTION OF THE INVENTION

The present invention provides pharmaceutical compositions comprising a therapeutically effective amount of an isolated interferon (IFN) or IFN mutein for use in the treatment of cardiomyopathy and treatment of endothelial dysfunction e.g., endothelial dysfunction connected to cardiomyopathy. Preferably, the isolated IFN is an isolated, human IFN and the IFN mutein is a variant of an isolated, human IFN. Examples of an isolated, human IFN of the present invention include, but are not limited to IFNα, IFNβ, (e.g., IFNβ-1a), and IFNγ. An example of an IFN mutein, include, but is not limited to, an IFNβ mutein, e.g., IFNβ-1b (also termed IFNβₛₑᵣ₁₇-1b, Betaseron®, Betaferon®), or an IFNα mutein. In some embodiments, the isolated IFN is an isolated, native, human IFNβ, and preferably, the IFN mutein is a variant of an isolated, native, human IFNβ. In other embodiments, the isolated IFN is an isolated, native, human IFNα, and preferably, the IFN mutein is a variant of an isolated, native, human IFNα.

The present invention further provides methods of treating cardiomyopathy and methods of treating endothelial dysfunction e.g., endothelial dysfunction connected to cardiomyopathy, using such a pharmaceutical composition comprising a therapeutically effective amount of an isolated IFN or IFN mutein. In some embodiments, the therapeutically effective amount of IFN or IFN mutein of the present invention is in a range that is from about 30 mcg to at least about 1000 mcg. In one embodiment, the therapeutically effective amount is in a range from about 30 mcg to about 500 mcg. In a preferred embodiment, the therapeutically effective amount is about 30 mcg. In another preferred embodiment, the therapeutically effective amount is about 250 mcg. In another preferred embodiment, the therapeutically effective amount is about 500 mcg.

The present invention provides pharmaceutical compositions and therapeutic methods that provide improved efficacy for treatment of a patient with cardiomyopathy and/or endothelial dysfunction e.g., endothelial dysfunction connected to cardiomyopathy, that are safe, well-tolerated, and demonstrate a positive trend towards beneficial effects for use in the treatment of patients with cardiomyopathy and/or endothelial dysfunction e.g., endothelial dysfunction connected to cardiomyopathy. Thus, the pharmaceutical compositions and methods of the present invention can increase the possibility of benefits from treatment of cardiomyopathy and treatment of endothelial dysfunction e.g., endothelial dysfunction connected to cardiomyopathy, using an IFNβ or IFNβ mutein and, also, can increase the number of patients that benefit from treatment.

The references cited herein, including journal articles, patents, and patent applications, are incorporated by reference, in their entirety.

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Standard reference works setting forth the general principles of recombinant DNA technology include Sambrook, J., et al. (1989) Molecular Cloning: A Laboratory Manual, 2d Ed., Cold Spring Harbor Laboratory Press, Planview, N.Y.; McPherson, M. J. Ed. (1991) Directed Mutagenesis: A Practical Approach, IRL Press, Oxford; Jones, J. (1992) Amino Acid and Peptide Synthesis, Oxford Science Publications, Oxford; Austen, B. M. and Westwood, O. M. R. (1991) Protein Targeting and Secretion, IRL Press, Oxford. Any suitable materials and/or methods known to those of ordinary skill in the art can be utilized in carrying out the present invention. However, preferred materials and methods are described. Materials, reagents and the like to which reference is made in the following description and examples are obtainable from commercial sources, unless otherwise noted.

Abbreviations
AE adverse event
BL baseline
BP blood pressure
BSA body surface area
CCS Canadian Cardiovascular Society
CD cluster determinant
CVB coxsackievirus B
CVC chronic viral cardiomyopathy
ECG electrocardiography
EF ejection fraction
ESR erythrocyte sedimentation rate
Fig. figure
FU follow up
GCP good clinical practice
GGT gamma-glutamyl transpeptidase
HARTS Hoechst Adverse Reaction Terminology System
HDL high density lipoproteins
HHV6 human herpes virus 6
HIV human immunodeficiency virus
HLA human leukocyte antigen system
HSA human serum albumin
HSA± human serum albumin free/containing
ICAM intercellular adhesion molecule
IFN interferon
INF interferon
INR international normalized ratio
ITT intention to treat
kD kilo Dalton
LDL low density lipoproteins
LV left ventricular
LVEDD left ventricular enddiastolic diameter
LVEF left ventricular ejection fraction
LVESD left ventricular endsystolic diameter
MIU million international units
mmHg millimeter mercury
NAB neutralizing antibody
nPCR nested polymerase chain reaction
NYHA New York Heart Association
PCR polymerase chain reaction
PE pretreatment event
PI patient information
PP per protocol
PT patient
PTS patients
rIFN recombinant interferon
RNV radionuclide ventriculography
SAE serious adverse event
s.c. subcutaneous

Treatment of Cardiomyopathy and of Endothelial Dysfunction

The pharmaceutical compositions and methods of the present invention are for use in the treatment of patients suffering from various clinically recognized forms of cardiomyopathy, including, but not limited to, e.g., non-specific cardiomyopathies and cardiomyopathies associated with specific cardiac or systemic disorders. Also, the pharmaceutical compositions and methods of the present invention are for use in the treatment of patients suffering from various clinically recognized forms of endothelial dysfunction, including, but not limited to, e.g., endothelial dysfunction connected to cardiomyopathy.

Treatment of patients suffering from cardiomyopathy associated with specific cardiac or systemic disorders, include those patients with, but not limited to, e.g., chronic inflammatory cardiomyopathy with or without the presence of virus, chronic viral cardiomyopathy, valvular cardiomyopathy, ischemic cardiomyopathy, hypertensive cardiomyopathy and having cardiac dysfunction resulting from, but are not limited to, e.g., myocardial virus presence, myocardial inflammation, coronary artery disease, heart valve disease, and arterial hypertension. Examples of non-specific cardiomyopathies include, but are not limited to, hypertrophic cardiomyopathy, restrictive cardiomyopathy, dilated cardiomyopathy and unclassified cardiomyopathies (see e.g., Circulation (1996) 93: 841-842).

Chronic inflammatory cardiomyopathy (myocarditis with cardiac dysfunction) is a focal or diffuse, chronic recurrent inflammatory process in the myocardium which may be induced, e.g., by a physical, chemical, and/or infectious agent. In many patients suffering from cardiomyopathy, Enteroviruses, especially group B Coxsackie-viruses, and Adenoviruses are the pathologic agents (see e.g., Kandolf, R. (1998) Med. Klin. 93: 215-222; Woodruff et al. (1980) Am. J. Pathol. 101: 425-484; Stille-Siegener et al. (1993) Idiopathic Dilated Cardiomyopathy. Springer-Verlag Berlin Heidelberg, Figulla et al. (Eds.), pp 369-372; Heim et al. (1 994) Clin. Cardiol. 17: 563-565). Moreover, Adenovirus, Enterovirus, Herpesvirus, and Parvovirus have been reported in endomyocardial biopsies of patients with cardiomyopathy (see e.g., Kandolf et al. (1993) Idiopathic Dilated Cardiomyopathy. Springer-Verlag Berlin Heidelberg, Figulla et al. (Eds.), pp. 267-283; Pauschinger et al. (1999) Circulation 99:889-895; Pauschinger et al. (1999) Circulation 99:1348-1354). However, clinically, cardiomyopathy may appear in a wide variety of forms, ranging from a total lack of clinical manifestations to progressive myocardial failure and sudden death.

Clinically isolated syndromes suggestive of cardiomyopathy include, but are not limited to, a general viral infection (e.g., hyposthenia, tiredness, and impairment of function) coincident with cardiac symptoms e.g., chest pain, arrhythmia and/or dyspnea, palpitations and syncopes due to arrhythmia, impairment of systolic or diastolic myocardial function with or without increase in ventricular size or disorder of a pace-setting or conduction system (see e.g., Schultheiss et al. (1998) Med. Klin. 93: 229-235), and precordial pain (e.g., as a result of raised filling pressure) which differs from a typical angina pectoris due to lack of exercise dependency (see e.g., Pankuweit et al. (1998) Med. Klin. 93: 223-228). Symptoms associated with cardiomyopathy also include, e.g., angina, fatigue, loss of strength, respiratory insufficiency, edema, interrupted sleep, and recurrent respiratory infection. For purposes of the present invention, the term “cardiomyopathy” is intended to encompass each of these clinical manifestations and associated symptoms of the disease and clinically isolated syndromes suggestive of cardiomyopathy unless otherwise specified.

As used herein, a “therapeutically effective dose” or “therapeutically effective amount” of an IFN or IFN mutein of the present invention is a dose or amount, when administered to a patient with cardiomyopathy as described herein, provides for treatment of cardiomyopathy and/or endothelial dysfunction e.g., endothelial dysfunction associated with cardiomyopathy.

As used herein, “treating” or “treatment” of cardiomyopathy and/or endothelial dysfunction using the pharmaceutical compositions and methods of the present invention result in an improvement in the respective disease or respective associated symptoms in a patient with cardiomyopathy and/or endothelial dysfunction. Thus, when a patient suffering from cardiomyopathy and/or endothelial dysfunction undergoes treatment in accordance with the pharmaceutical compositions and methods of the present invention, treatment can result in the prevention and/or amelioration of cardimyopathy and/or endothelial dysfunction disease symptoms, disease severity, and/or recurrence of the disease, i.e., treatment of cardiomyopathy cardiomyopathy and/or endothelial dysfunction using the compositions and methods of the present invention can result in lengthening the time period between episodes in which symptoms flare, and/or can suppress the symptoms associated with the respective disease, which, left untreated, can enhance disease progression and disability.

As used herein “patient” refers to a subject, preferably a human, who is in need of treatment. For example, a subject having cardiomyopathy or symptoms associated with cardiomyopathy is a patient in need of treatment of cardiomyopathy or associated symptoms of cardiomyopathy. Also, for example, a subject having endothelial dysfunction or symptoms associated with endothelial dysfunction is a patient in need of treatment of endothelial dysfucntion or associated symptoms of endothelial dysfucntion.

Factors influencing the amount of an IFN or IFN mutein of the present invention that constitutes a therapeutically effective dose include, but are not limited to, the severity of the disease (i.e., cardiomyopathy and/or endothelial dysfunction e.g., endothelial dysfunction connected to cardiomyopathy), the history of the disease, and the age, health, and physical condition of the individual undergoing therapy. Generally, a higher-dose of an IFN or IFN mutein of the present invention is preferred as tolerated and safe. In preferred embodiments, the IFN is IFNβ and the IFN mutein is an IFNβ mutein.

As used herein, a therapeutically effective amount of an IFN or IFN mutein of the present invention refers to an amount of the IFN or IFN mutein that is in a range that is from about 30 mcg to at least about 1000 mcg.

In some embodiments, the therapeutically effective amount of the IFN or IFN mutein of the present invention is in a range from about 30 mcg to about 500 mcg. For example, in some embodiments the therapeutically effective amount is about 30 mcg to about 50 mcg, about 50 mcg to about 75 mcg, about 75 mcg to about 100 mcg, or about 100 mcg to about 125 mcg, about 125 mcg to about 150 mcg, about 150 mcg to about 175 mcg, about 175 mcg to about 200 mcg, about 200 mcg to about 225 mcg, about 225 mcg to about 250 mcg, about 250 mcg to about 275 mcg, about 275 mcg to about 300 mcg, about 300 mcg to about 325 mcg, about 325 mcg to about 350 mcg, about 350 mcg to about 375 mcg, about 375 mcg to about 400 mcg, about 400 mcg to about 425 mcg, about 425 mcg to about 450 mcg, about 450 mcg to about 475 mcg, or about 475 mcg to about 500 mcg. In a preferred embodiment, the therapeutically effective amount is about 30 mcg. In another preferred embodiment, the therapeutically effective amount is about 250 mcg. In another preferred embodiment, the therapeutically effective amount is about 500 mcg.

In some embodiments, the therapeutically effective amount of the IFN or IFN mutein of the present invention is in a range that is at least about 500 mcg to at least about 1000 mcg. For example, in some embodiments, the therapeutically effective amount of IFN or IFN mutein is at least about 500 mcg to at least about 525 mcg, at least about 525 mcg to at least about 550 mcg, at least about 550 mcg to at least about 575 mcg, at least about 575 mcg to about at least about 600 meg, at least about 600 mcg to at least about 625 mcg, at least about 625 mcg to at least about 650 mcg, at least about 650 mcg to at least about 675 mcg, at least about 675 mcg to at least about 700 mcg, at least about 700 mcg to at least about 725 mcg, at least about 725 mcg to at least about 750 mcg, at least about 750 mcg to at least about 775 mcg, at least about 800 mcg to at least about 825 mcg, at least about 825 mcg to at least about 850 mcg, at least about 850 mcg to at least about 875 mcg, at least about 875 mcg to at least about 900 mcg, at least about 900 mcg to at least about 925 mcg, at least about 925 mcg to at least about 950 mcg, at least about 950 mcg to at least about 975 mcg, or at least about 975 mcg to at least about 1000 mcg.

Further, a therapeutically effective dose of an IFN or IFN mutein of the present invention can also depend upon the dosing frequency and severity of the disease in the patient undergoing treatment who has cardiomyopathy and/or endothelial dysfunction e.g., endothelial dysfunction connected to cardiomyopathy. Preferably, the IFN is an IFNβ or an IFNα, and the IFN mutein is an IFNβ mutein or an IFNα mutein.

In a preferred embodiment, the therapeutically effective dose of an IFN or IFN mutein of the present invention can be administered subcutaneously with a dosing frequency of every other day. In another embodiment, the dosing frequency can be once to twice a week, three to four times a week, or five to six times a week, or daily.

The dosing regimen can be continued for as long as is required to achieve the desired effect, i.e., for example amelioration of the disease, symptoms associated with the disease, disease severity, and/or periodicity of the recurrence of the disease, as described herein. In one embodiment, the dosing regimen is continued for a period of up to one year to indefinitely, such as for one month to 30 years, about three months to about 20 years, about 6 months to about 10 years.

Symptoms of cardiomyopathy and/or endothelial dysfunction e.g., endothelial dysfunction connected to cardiomyopathy, that are prevented, ameliorated, or treated, when a patient undergoes therapy in accordance with the methods of the present invention include those described herein and known in the art. A patient having cardiomyopathy and/or endothelial dysfunction e.g., endothelial dysfunction connected to cardiomyopathy, may have one or more of the symptoms associated with cardiomyopathy and/or endothelial dysfunction e.g., endothelial dysfunction connected to cardiomyopathy, and one or more can be ameliorated by the pharmaceutical compositions and methods of the present invention.

The pharmaceutical compositions of the present invention can also block the pathophysiological pathway associated with cardiomyopathy and/or endothelial dysfunction e.g., endothelial dysfunction connected to cardiomyopathy. Beneficial effects of the pharmaceutical compositions and methods of the present invention include, e.g., slowing the onset of the established disease, ameliorating symptoms of the disease, slowing the progression of the disease, or reducing the appearance of the symptoms of the disease, and postponing or preventing disability caused by the disease.

Adverse effects due to some cardiomyopathy and/or endothelial dysfunction e.g., endothelial dysfunction connected to cardiomyopathy, treatment regimens are known in the art and described herein. For example, some of the adverse effects due to treatment of cardiomyopathy and/or endothelial dysfunction e.g., endothelial dysfunction connected to cardiomyopathy, include, but are not limited to, those described herein in Example 1. Suitable co-medications and the use of these co-medications for treating adverse effects due to treatment of cardiomyopathy and/or endothelial dysfunction e.g., endothelial dysfunction connected to cardiomyopathy, can be determined according to co-medications generally known in the art for treatment of such effects. Doses and dosing regimens for such co-medications are also generally known. Examples of such co-medications include, but are not limited to, analgesics, steroids, and non-steroidal anti-inflammatory drugs (NSAIDs).

Suitable examples of co-medications also include, but are not limited to, e.g., ibuprofen, acetaminophen, acetylsalicyclic acid, prednisone, pentoxifylline, baclofen, steroids, antibacterial agents, and antidepressants (see e.g., Walther et al. (1999) Neurology 52: 1622-1627). For example, flu-like symptoms can be treated with NSAIDs (e.g., ibuprofen or acetylsalicylic acid) or with paracetamol or with pentoxifylline; injection site reactions can be treated with systemic NSAIDs and/or steroids (e.g., hydrocotisone); cutaneous or subcutaneous necrosis can be treated with antibacterial agents and depression can be treated with antidepressants (see e.g., Walther et al. (1999) Neurology 53: 1622-1627).

Combination therapies with the pharmaceutical compositions and methods of the present invention and other drugs and therapies which are effective in the treatment of cardiomyopathy and/or endothelial dysfunction e.g., endothelial dysfunction connected to cardiomyopathy, and have a different adverse event, may increase the treatment effect and level out the adverse event profile. Suitable examples of combination therapies include, but are not limited to, e.g., glatiramer acetate (Copaxone), mitoxantrone, cyclophosphamide, cyclosporine A, cladribine, monoclonal antibodies (e.g., Campath-H1® or Antegren®/Natazulimab®), and statins.

Standard known therapies for treatment of e.g., dilated cardiomyopathy consist of e.g., ACE inhibitor, diuretic, digitalis, β-blocker, aldosterone antagonist, anticoagulant, and/or antiarrhythmic drug which may also be useful in combination therapies with the pharmaceutical compositions and methods of the present invention.

Effective treatment of cardiomyopathy in a patient using the methods of the invention can be examined in using methods known in the art and those described herein. Such methods can be used for the evaluation and monitoring of treatment of cardiomyopathies and include, but are not limited to, e.g., chest roentgenogram, electrocardiogram, echocardiogram, radionuclide studies, cardiac catheterization, and detection of antibodies associated with cardiomyopathy (see e.g., U.S. Pat. Ser. No. 5,418,139).

Tentative diagnosis of viral cardiomyopathy may be given when symptoms of a general viral infection (hyposthenia, tiredness and impairment of functional capacity) coincide with atypical cardiac symptoms (chest pain, arrhythmia) or progressive dyspnea in formerly healthy individuals. The cardiac symptoms indicate impairment of systolic or diastolic myocardial function with or without increase in ventricular size or disorder of the pace-setting or conduction system. Patients mainly complain about symptoms of heart failure (e.g. fatigue, dyspnea).

Virus persistence may result in other symptoms indicative of cardiomyopathy, including, but not limited to, e.g., the further loss of myocytes, hypertrophy and degeneration of the myocardial contractile apparatus, leading to myocardial fibrosis and myocardial dysfunction. Besides necrobiosis of myocytes due to acute virus replication and virus-induced inflammatory processes, the virus seems to be a causative agent for progressive cardiac damage due to a further effect. In vivo studies indicate that dystrophin and the dystrophin-associated glycoproteins are morphologically disrupted in infected myocytes, leading to impaired dystrophin function. Impaired dystrophin function may contribute to the pathogenesis of acquired dilated cardiomyopathy which in the final stage requires heart transplantation. Consequently, impaired dystrophin function may also be a symptom indicative of cardiomyopathy.

In many patients suffering from cardiomyopathy, Enteroviruses, especially group B Coxsackie-viruses, and Adenoviruses are the pathologic agents (see e.g., Kandolf, R. (1998) Med. Klin. 93: 215-222; Woodruff et al. (1980) Am. J. Pathol. 101: 425-484; Stille-Siegener et al. (1993) Idiopathic Dilated Cardiomyopathy. Springer-Verlag Berlin Heidelberg, Figulla et al. (Eds.), pp 369-372; Heim et al. (1994) Clin. Cardiol. 17: 563-565). Consequently, the diagnosis of some cardiomyopathies, e.g., idiopathic dilated cardiomyopathy, can be confirmed by biopsy, and the presence of cardiotropic virus can be evaluated measuring virus neutralizing antibody titers in blood (see e.g., Miric et al. (1996) Heart 75: 596-601). Moreover, molecular biological techniques can be use to detect viruses (e.g., Adenovirus, Enterovirus, Herpesvirus, and Parvovirus) in endomyocardial biopsies of patients with cardiomyopathy (see e.g., Kandolf et a. (1993) Idiopathic Dilated Cardiomyopathy. Springer-Verlag Berlin Heidelberg, Figulla et al. (Eds.), pp. 267-283; Pauschinger et al. (1999) Circulation 99:889-895; Pauschinger et al. (1999) Circulation 99:1348-1354).

As mentioned, the pharmaceutical compositions and methods of the present invention are for use in the treatment of patients suffering from various clinically recognized forms of endothelial dysfunction, including, but not limited to, e.g., endothelial dysfunction connected to cardiomyopathy.

Endothelium dependent flow-mediated vasodilation of systemic arteries was found to be impaired in patients with non-ischemic cardiomyopathy and virus persistence. (see Vallbracht et al. (2004) Circulation 110: 2938-2945). In some cases and to different degrees this may be associated with inflammatory signs and is also seen in the coronary microcirculation ( see Vallbracht et al. (2002) JACC 40.3: 510-520, Vallbracht et al. (2004) Circulation 111: 1784-1791). Presence of virus may also be possible in patients who have cardiomyopathy due to other reasons.

Viral mediated, in particular parvovirus mediated endothelial dysfunction can be considered a relevant factor in the pathology of cardiomyopathy as decribed herein.

Therapeutic agents in heart failure therapy, including the therapy of cardiomyopathy, atherosclerosis and hypertension target also endothelial dysfunction. For example, long established therapy with ACE inhibitors reduces elevated serum markers in hypertensive patients but do not affect their level in normal controls. This may be mediated by reducing damaging factors to the endothelium ba the Renin-angiotensin-bradykinin system.

Infectious agents were thought to be associated with atherosclerosis. Herpesviridae, Chlamydia pneumoniae or Helicobacter appear to be linked in a number of studies but their pathogenic role, or that of a bystander, is not clear and there is negative precedence of antimicrobial therapy that could have strenghtened the pathogenesis link. A therapeutic benefit can be derived if studies with an antiviral and immunomudulatory therapy with an interferon showed a reduction of virus or its elimination in cardiac patients, particularly including the parvovirus that is dwelling in the endothelial cells and vascular wall.

Interferon can improve management of patients with conditions that are due to, are leading to, or are significantly associated with endothelial dysfunction, like cardiomyopathy but also arterial hypertension or atherosclerosis, by an anti-inflammatory and antiviral mechanism and by contributing to restoration of normal endothelial function, particularly when endothelial function is altered by viral (Parvovirus B19) affection. In the latter case a reduction of virus load would contribute to a causal therapy.

In case an otherwise non-recognized infection of the endothelium or elements of the vascular wall were recognized in the rank of risk factor, or significant contributing factor, for cardiac or vascular disease, there would be a rationale for diagnosing and treating this condition early, when recognized.

A study of an interferon therapy in patients who suffer from cardiomyopathy with evidence of presence of viral genome in the heart or in patients with inflammatory findings in the heart or peripheral vessels and who have an endothelial dysfunction can demonstrate improvement the clinical condition and heart function, as well as in repeated assessments of endothelial function and would therewith show the therapeutic benefit that would make such a therapy eligible for cardiomyopathy in which a coronary and capillary endothelial dysfunction contributes significantly, for example as can be assumed for parvovirus associated cardiomyopathy, and such therapy can also be eligible for other vascular or microcirculatory conditions in which endothelial dysfunction is a pathogeneic element or symptom.

The beneficial effect of the interferon therapy can be demonstrated by a wide range of variables. These include improvements in the assessments of symptoms and markers of of cardiomyopathy as decribed herein, direct measurement of the Flow Mediated Dysfunction (FMD) of the vascular wall compared to the endothelium independent vasoreactivity (e.g., GTN-MD), improvements of circulating or vascular inflammatory markers ( e.g., e-selectin, adhesion molecules), improvements of thrombogenicity of the vascular wall—for example measured by thrombus formation or indirect indicators, reduced vascular wall remodeling and other therapeutic or diagnostic variables or markers common in arterial hypertension or atherosclerosis by which endothelial dysfunction is assessed.

Interferons

The present invention provides pharmaceutical compositions comprising a therapeutically effective amount of an isolated IFN or IFN mutein that is a variant of an IFN. Examples of suitable IFN for use in the pharmaceutical compositions of the present invention include, but are not limited to, e.g., IFNα, IFNβ, and IFNγ. Preferably, the IFN is a human IFN and more preferably a native, human IFN. In some embodiments, the IFN is a native, human IFNβ or a native, human IFNα. In one embodiment, the IFNβ is IFNβ-1a.

Interferons are well known and can be isolated, produced, and tested for activity using known methods in the art. For example, the sequence of an IFN or IFN mutein suitable for use in the compositions and methods of the present invention can be of, or derived from, a native IFN gene sequence (or other nucleic acid sequence encoding an IFN, e.g., an IFN RNA sequence) or IFN protein sequence of e.g., a vertebrate, preferably a mammal, and most preferably a human, and can be obtained using methods well-known in the art e.g., as described e.g., in U.S. Pat. Ser. Nos. 4,727,138 and 4,970,161 (human IFN-γ); U.S. Pat. No. 5,071,761 (human lymphoblastoid interferons LyIFNα-2 and LyIFNα-3); U.S. Pat. No. 4,695,543 (human IFNα-Gx-1). Examples of such suitable nucleic acid sequences encoding an IFN include, but are not limited to: SEQ ID NO: 3 (human IFN-γ); SEQ ID NO: 5 (human IFNα-1); SEQ ID NO: 7 (human IFNα-2); SEQ ID NO: 9 (IFNα-4); SEQ ID NO: 11 (human IFNα-5); SEQ ID NO: 13 (human IFNα-6); SEQ ID NO: 15 (human IFNα-7); SEQ ID NO: 17 (human IFNα-8); SEQ ID NO: 19 (human IFNα-10); SEQ ID NO: 21 (human IFNα-13); SEQ ID NO: 23 (human IFNα-14); SEQ ID NO: 25 (human IFNα-16); SEQ ID NO: 27 (human IFNα-17); SEQ ID NO: 29 (human IFNα-21); SEQ ID NO: 31 (mouse IFNα-2); and SEQ ID NO: 33 (mouse IFNβ3). Examples of such suitable IFN amino acid sequences include, but are not limited to: SEQ ID NO: 1 (human IFNβ-1a); SEQ ID NO: 2 (human IFNβ-1b); SEQ ID NO: 4 (human IFN-γ); SEQ ID NO: 6 (human IFNα-1); SEQ ID NO: 8 (human IFNα-2); SEQ ID NO: 10 (IFNα-4); SEQ ID NO: 12 (human IFNα-5); SEQ ID NO: 14 (human IFNα-6); SEQ ID NO: 16 (human IFNα-7); SEQ ID NO: 18 (human IFNα-8); SEQ ID NO: 20 (human IFNα-10); SEQ ID NO: 22 (human IFNα-13); SEQ ID NO: 24 (human IFNα-14); SEQ ID NO: 26 (human IFNα-16); SEQ ID NO: 28 (human IFNα-17); SEQ ID NO: 30 (human IFNα-21); SEQ ID NO: 32 (mouse IFNα-2); and SEQ ID NO: 34 (mouse IFNβ).

Recombinant DNA methods for producing proteins, including interferons are known (see e.g., U.S. Pat. Nos. 4,399,216; 5,149,636; 5,179,017; and 4,470,461). Further, cysteine-depleted variants to minimize formation of unwanted inter- or intra-molecular disulfide bonds (see e.g., U.S. Pat. Nos. 4,518, 584;, 4,588,585;, 4,959,314) and, also, methionine-depleted variants to minimize susceptibility to oxidation, have been reported (see e.g., EPO No. 260350).

Further, interferons with modified activity have been reported (see e.g., U.S. Pat. Nos. 6,514,729; 4,738,844; 4,738,845; 4,753,795; 4,766,106; WO 00/78266). Also, substitution mutants of IFNβ at position 101 have been reported (see e.g., U.S. Pat. Nos. 5,545,723 and 6,127,332). Chimeric interferons comprising sequences from one or more interferons have also been reported (see e.g., Chang et. al. Nature Biotech. 17: 793-797 (1999), U.S. Pat. Nos. 4, 758,428; 4,885,166; 5,382,657; 5,738,846). Substitution mutations to IFNβ at positions 49 and 51 have also been reported (see e.g., U.S. Pat. No. 6,531,122). Further, IFNβ variants with enhanced stability have been reported, in which the hydrophobic core was optimized using rational design methods (see e.g., WO 00/68387). Alternate formulations that promote interferon stability or solubility have also been reported (see e.g., U.S. Pat. Nos. 4,675,483; 5,730, 969; 5,766,582; and PCT Application No. WO 02/38170). Also reported are IFNβ muteins with enhanced solubility have been claimed, in which several leucine and phenylalanine residues are replaced with serine, threonine, or tyrosine residues (see e.g., WO 98/48018). IFNα and IFNβ variants with reduced immunogenicity have also been reported (see e.g., WO 02/085941 and WO 02/074783). Further, it has been reported that human fibroblast interferon has antiviral activity, and is a polypeptide of about 20,000 Daltons induced by viruses and double-stranded RNAs. From the nucleotide sequence of the gene for fibroblast interferon, cloned by recombinant DNA technology, Derynk et al. (Derynk R. et al., Nature 285, 542-547, 1980) reportedly deduced the amino acid sequence of the protein.

Further, recombinant interferon, for example a recombinant IFNα (e.g., 2a or 2b, IFNβ (e.g., 1a or 1b), or IFNγ, have been cloned and expressed in E. coli (e.g., Weissmann et al., Science, 209:1343-1349 (1980); Sreuli et al., Science, 209:1343-1347 (1980); Goeddel et al., Nature, 290:20-26 (1981); Henco et al., J. Mol. Biol., 185:227-260 (1985)). In some embodiments, the interferon is a human IFNβ-1a or IFNβ-1b; or a human IFNα-2a or IFNα-2b (see e.g., WO 91/18927); or a human IFNγ. In some embodiments, the inteferon has anti-viral, anti-cancer, anti- infective, and/or immune modulating activity. In some embodiments the administered interferon protein is a semisynthetic protein-polymer conjugate (e.g., a PEGylated interferon). In one embodiment the IFNα is a PEGylated human IFNα-2b. In another embodiment the IFNβ is a PEGylated human IFNβ-1a or IFNβ-1b. A 12,000-Da monomethoxypolyethylene glycol (PEG-12000) polymer can be attached. PEG conjugation is thought to increase the serum half-life and thereby prolong patient exposure to the administered IFN protein without altering the biologic potency of the protein.

In preferred embodiments, the pharmaceutical composition of the present invention comprises a therapeutically effective amount of a native, human IFNβ or an IFNβ mutein that is a variant of a native, human IFNβ. Native, human IFNβ is a regulatory polypeptide with a molecular weight of 22 kDa consisting of 166 amino acid residues (see e.g., SEQ ID NO: 1). The polypeptide can be produced by most cells in the body, in particular fibroblasts, in response to viral infection or exposure to other biologics. Further, IFNβ binds to a multimeric cell surface receptor, and productive receptor binding results in a cascade of intracellular events leading to the expression of IFNB inducible genes which in turn produces effects which can be classified as antiviral, antiproliferative and immunomodulatory.

Human IFNβ is a well-characterized polypeptide. The amino acid sequence of human IFNβ is known (see e.g., Gene 10:11-15,1980, and in EP 83069, EP 41313and U.S. Pat. No. 4,686,191). Also, crystal structures have been reported for human and murine IFNβ, respectively (see e.g., Proc. Natl. Acad. Sci. USA 94:11813-11818,1997. J. Mol. Biol. 253:187-207,1995; reviewed in Cell Mol. Life Sci. 54:1203-1206, 1998). In addition, protein-engineered variants of IFNβ have been reported (see e.g., WO 9525170, WO 9848018, U.S. Pat. No. 5,545,723, U.S. Pat. No. 4,914,033, EP 260350, U.S. Pat. No. 4,588,585, U.S. Pat. No. 4,769,233, Stewart et al, DNA Vol. 6 No. 2 1987 pp. 119-128, Runkel et al, 1998, Jour. Biol. Chem. 273, No. 14, pp. 8003-8008). Also, the expression of IFNβ in CHO cells has been reported (see e.g., U.S. Pat. No. 4,966,843, U.S. Pat. No. 5,376,567 and U.S. Pat. No. 5,795,779).

Further, IFNβ molecules with a particular glycosylation pattern and methods for their preparation have been reported (see e.g., EP 287075 and EP 529300). Also reported is the modification of polypeptides by polymer conjugation or glycosylation. For example, polymer modification of native IFNβ or a C17S variant thereof has been reported (see e.g., EP 229108, U.S. Pat. No. 5,382,657, EP 593868, U.S. Pat. No. 4,917,888 and WO 99/55377). PEGylated lysine depleted polypeptides have also been reported, wherein at least one lysine residue has been deleted or replaced with any other amino acid residue (see e.g., U.S. Pat. No. 4,904,584). Further processes for conjugating a protein with PEG have been reported (see e.g., WO 99/67291), wherein at least one amino acid residue on the protein is deleted and the protein is contacted with PEG under conditions sufficient to achieve conjugation to the protein.

PEGylated variants of polypeptides belonging to the growth hormone superfamily have also been reported, wherein a cysteine residue has been substituted with a non-essential amino acid residue located in a specified region of the polypeptide and IFNβ has been reported as one example of a polypeptide belonging to the growth hormone superfamily (see e.g., WO 99/03887). Glycosylated and PEGylated IFNβ are reported e.g., in WO 00/23114. WO 00/26354 reports a method of producing a glycosylated polypeptide variant with reduced allergenicity, which as compared to a corresponding parent polypeptide comprises at least one additional glycosylation site.

Also reported is the modification of granulocyte colony stimulating factor (G-CSF) and other polypeptides so as to introduce at least one additional carbohydrate chain as compared to the native polypeptide (see e.g., U.S. Pat. No. 5,218,092). IFNβ is mentioned as one example among many polypeptides that allegedly can be modified according to the technology described in U.S. Pat. No. 5,218,092.

Further, IFNβ fusion proteins are reported, e.g., in WO 00/23472.

Commercial preparations of IFNβ or IFNβmutein are approved for the treatment of patients with multiple sclerosis and sold under the names Betaseron® (also termed Betaferon® or IFNβ-1b_{ser17,} which is non-glycosylated IFNβ mutein, produced using recombinant bacterial cells, has a deletion of the N-terminal methionine residue and the C17S mutation), Avonex® and Rebif® (also termed IFNβ-1a, which is glycosylated, produced using recombinant mammalian cells. Further, a comparison of IFNβ-1a and IFNβ-1b with respect to structure and function has been presented in Pharm. Res. 15:641-649, 1998.

IFNβ has inhibitory effects on the proliferation of leukocytes and antigen presentation. Furthermore, IFNβ may modulate the profile of cytokine production towards an anti-inflammatory phenotype. Finally, IFNβ can reduce T-cell migration by inhibiting the activity of T-cell matrix metalloproteases. Such IFNβ activities are likely to act in concert to account for the beneficial effect of IFNβ in the treatment of patients with cardiomyopathy and/or endothelial dysfunction e.g., endothelial dysfunction connected to cardiomyopathy.

Interferon Muteins

As used herein, “IFN mutein” refers to variants of a native IFN, and can also be referred to as IFN-like polypeptides. For example, the IFN muteins of the present invention can be variants of an IFNα, IFNβ, or IFNγ. Preferably, the IFN mutein is a human IFN mutein and more preferably is a human IFNβ mutein or a human IFNα mutein. In a preferred embodiment, the human IFNβ mutein is IFNβ-1b (also termed IFNβₛₑᵣ₁₇-1b, Betaseron®, Betaferon®). Variants of a native, human IFN (e.g., IFNβ), which may be naturally occurring (e.g., allelic variants that occur at an IFNβ locus) or recombinantly or synthetically produced, have amino acid sequences that are similar to, or substantially similar to a mature, native IFN sequence. The IFN muteins of the present invention also encompass fragments of an IFN or truncated forms of an IFN that retain IFN activity. These biologically active fragments or truncated forms of an IFN can be generated by removing amino acid residues from the full-length IFN amino acid sequence using recombinant DNA techniques well known in the art. Also, the IFN or IFN muteins of the present invention may be glycosylated or not glycosylated.

An example of an amino acid sequence of a mature, native, human IFNβ is SEQ ID NO: 1. In a preferred embodiment, the IFNβ mutein of the present invention is a variant of a mature, native, human IFNβ sequence, wherein one or more cysteine residues that are not essential to IFNβ biological activity have been deliberately deleted or replaced with other amino acids to eliminate sites for either intermolecular crosslinking or incorrect intramolecular disulfide bond formation (e.g., IFNβ-1b). IFNβ muteins of this type include those containing a glycine, valine, alanine, leucine, isoleucine, tyrosine, phenylalanine, histidine, tryptophan, serine, threonine, or methionine substituted for the cysteine found at amino acid 17 of the mature, native IFNβ amino acid sequence. Serine and threonine are the more preferred replacements because of their chemical analogy to cysteine. Serine substitutions are most preferred. An example of an amino acid sequence of an IFNβ mutein of the present invention is SEQ ID NO: 2. In a preferred embodiment, the IFNβ mutein is Betaseron® (see e.g., U.S. Pat. Nos 4,588,585; 4,959,314; 4,737,462; L. Lin (1998) Dev. Biol. Stand. 96: 97-104).

In one embodiment, the cysteine found at amino acid 17 of the mature native sequence is replaced with serine. Cysteine 17 may also be deleted using methods known in the art (see, for e.g., U.S. Pat. No. 4,588,585), resulting in an IFNβ mutein that is one amino acid shorter than the mature native IFNβ (see also, e.g., U.S. Pat. Nos. 4,530,787; 4,572,798; and 4,588,585). Thus, IFN muteins (e.g., IFNβ-1b) with one or more mutations that improve the therapeutic utility of an IFN are encompassed by the present invention.

Additional changes can be introduced by mutation into the nucleotide sequences encoding an IFN, thereby leading to changes in the IFN amino acid sequence, without altering the biological activity of the interferon. Thus, an isolated nucleic acid molecule encoding, e.g., an IFNβ mutein having a sequence that differs from the amino acid sequence for the mature, native IFNβ can be created by introducing one or more nucleotide substitutions, additions, or deletions into the corresponding nucleotide sequence (see, e.g., U.S. Pat. No. 5,588,585), such that one or more amino acid substitutions, additions or deletions are introduced into the encoded IFNβ. Mutations can be introduced by standard techniques, such as site-directed mutagenesis and PCR-mediated mutagenesis. Such IFN muteins are also encompassed by the present invention.

As an example, conservative amino acid substitutions may be made at one or more predicted, preferably nonessential amino acid residues. As used herein, a “nonessential” amino acid residue is a residue that can be altered from the wild-type sequence of an IFN without altering its biological activity, whereas an “essential” amino acid residue is required for the biological activity of the IFN. As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine). In preferred embodiments, such substitutions are not made for conserved amino acid residues, or for amino acid residues residing within a conserved motif.

Alternatively, IFN mutein nucleotide sequences can be made by introducing mutations randomly along all or part of an IFN coding sequence (e.g., IFNβ coding sequence), such as by saturation mutagenesis, and the resultant mutants can be screened for IFN biological activity (e.g., IFNβ biological activity) to identify mutants that retain such IFN activity. Following mutagenesis, the encoded protein can be expressed recombinantly, and the activity of the protein can be determined using standard assay techniques described herein.

In preferred embodiments, biologically active IFN muteins of the present invention have at least 80%, more preferably about 90% to about 95% or more, and most preferably about 96% to about 99% or more amino acid sequence identity to the amino acid sequence of a mature, native IFN, which serves as the basis for comparison or reference. As used herein “sequence identity” is the same amino acid residues that are found within the variant polypeptide and the polypeptide molecule that serves as a reference when a specified, contiguous segment of the amino acid sequence of the variant is aligned and compared to the amino acid sequence of the reference molecule.

For the optimal alignment of two sequences for the purposes of sequence identity determination, the contiguous segment of the amino acid sequence of the mutein may have additional amino acid residues or deleted amino acid residues with respect to the amino acid sequence of the reference molecule. The contiguous segment used for comparison to the reference amino acid sequence will comprise at least 20 contiguous amino acid residues. Corrections for increased sequence identity associated with inclusion of gaps in the amino acid sequence of the mutein can be made by assigning gap penalties. Methods of sequence alignment are well known in the art.

For example, the determination of percent identity between any two sequences can be accomplished using a mathematical algorithm. One preferred, non-limiting example of a mathematical algorithm utilized for the comparison of sequences is e.g., the algorithm of Myers and Miller (1988) Comput. Appl. Biosci. 4:11-7. Such an algorithm is utilized in the ALIGN program (version 2.0), which is part of the GCG alignment software package. A PAM120 weight residue table, a gap length penalty of 12, and a gap penalty of 4 can be used with the ALIGN program when comparing amino acid sequences. Another preferred, non-limiting example of a mathematical algorithm for use in comparing two sequences is the algorithm of Karlin and Altschul (1990) Proc. Natl. Acad. Sci. USA 90:5873-5877, modified as in Karlin and Altschul (1993) Proc. Natl. Acad. Sci USA 90:5873-5877. Such an algorithm is incorporated into the NBLAST and XBLAST programs of Altschul et al. (1990) J. Mol. Biol. 215:403-410. BLAST amino acid sequence searches can be performed with the XBLAST program, score=50, wordlength=3, to obtain amino acid sequence similar to the polypeptide of interest.

To obtain gapped alignments for comparison purposes, gapped BLAST can be utilized as described in Altschul et al. (1997) Nucleic Acids Res. 25:3389-3402. Alternatively, PSI-BLAST can be used to perform an integrated search that detects distant relationships between molecules (see e.g., Altschul et al. (1997) supra.). When utilizing BLAST, gapped BLAST, or PSI-BLAST programs, the default parameters can be used (see e.g., www.ncbi.nlm.nih.gov). Also see the ALIGN program (Dayhoff (1978) in Atlas of Protein Sequence and Structure 5:Suppl. 3, National Biomedical Research Foundation, Washington, D.C.) and programs in the Wisconsin Sequence Analysis Package, Version 8 (available from Genetics Computer Group, Madison, Wis.), for example, the GAP program, where default parameters of the programs are utilized.

When considering percentage of amino acid sequence identity, some amino acid residue positions may differ as a result of conservative amino acid substitutions, which do not affect properties of protein function. In these instances, percent sequence identity may be adjusted upwards to account for the similarity in conservatively substituted amino acids. Such adjustments are well known in the art (see, e.g., Myers and Miller (1988) Comput. Appl. Biosci. 4:11-17).

Biologically active IFN or IFN muteins encompassed by the present invention also include IFN or IFN muteins that are covalently linked with, e.g., polyethylene glycol (PEG) or albumin. These covalent hybrid IFN or IFN mutein molecules can have certain desirable pharmaceutical properties such as an extended serum half-life after administration to a patient. For example, PEG can be used to impart water solubility, size, slow rate of kidney clearance, and reduced immunogenicity to the fusion protein (see e.g., U.S. Pat. No. 6,214,966). Such IFN or IFN muteins of the present invention having PEG fused thereto are called herein “PEGylated IFN” or “PEGylated IFN muteins”, respectively.

Methods for creating PEG-IFN adducts involve chemical modification of monomethoxypolethylene glycol to create an activated compound that will react with an IFN or IFN mutein. Methods for making and using PEG-linked polypeptides are reported, e.g., in Delgado et al. (1992) Crit. Rev. Ther. Drug. Carrier Syst. 9:249-304 (and as described herein in the Background). Methods for creating albumin fusion polypeptides involve fusion of the coding sequences for the polypeptide of interest (e.g., IFN or IFN mutein) and albumin and are reported, e.g., in U.S. Pat. No. 5,876,969. Methods known in the art can be used to covalently link or fuse compounds to the IFN or IFN muteins of the present invention.

Biologically active IFN or IFN muteins encompassed by the present invention also include, e.g., fusion proteins that have been fused with another compound, such as a compound to increase the half-life of the polypeptide and/or to reduce potential immunogenicity of the polypeptide. Methods known in the art for making fusion proteins can be used to make the IFN fusion protein or IFN mutein fusion protein of the present invention.

Biologically active IFN muteins (e.g., IFNβ muteins or IFNα muteins) encompassed by the invention preferably retain IFN activities, particularly the ability to bind to cognate IFN receptors. In some embodiments, the IFN mutein retains at least about 25%, about 50%, about 75%, about 85%, about 90%, about 95%, about 98%, about 99% or more of the biologically activity of the reference IFN polypeptides. IFN muteins whose activity is increased in comparison with the activity of the reference polypeptides are also encompassed. The biological activity of IFN variants can be measured by any method known in the art (see e.g., assays described in Fellous et al. (1982) Proc. Natl. Acad. Sci USA 79:3082-3086; Czerniecki et al. (1984) J. Virol. 49(2):490-496; Mark et al. (1984) Proc. Natl Acad. Sci. USA 81:5662-5666; Branca et al. (1981) Nature 277:221-223; Williams et al. (1979) Nature 282:582-586; Herberman et al. (1979) Nature 277:221-223; Anderson et al. (1982) J. Biol. Chem. 257(19):11301-11304).

Suitable IFN muteins for use in the pharmaceutical compositions and methods of the present invention can be variants of a native IFN of any mammalian species including, but not limited to, avian, canine, bovine, porcine, equine, and human. Preferably, the IFN mutein of the present invention is a variant of a native human IFN, in either its glycosylated or unglycosylated form. More preferably, the IFN mutein of the present invention is a variant of a human IFN. In some embodiments, the IFN mutein of the present invention is a variant of a native, human IFNβ or a variant of a native, human IFNα. In one embodiment, the IFN mutein is IFNβ-1b.

Non-limiting examples of IFN muteins encompassed by the invention are set forth in, e.g., Nagata et al. (1980) Nature 284:316-320; Goeddel et al. (1980) Nature 287:411-416; Yelverton et al. (1981) Nucleic Acids Res. 9:731-741; Streuli et al. (1981) Proc. Natl. Acad. Sci. U.S.A. 78:2848-2852; EP028033B1, and EP109748B1. See also, e.g., U.S. Pat. Nos. 4,518,584; 4,569,908; 4,588,585; 4,738,844; 4,753,795; 4,769,233; 4,793,995; 4,914,033; 4,959,314; 5,545,723; and 5,814,485. These citations also provide guidance regarding residues and regions of IFN polypeptides that can be altered without the loss of biological activity.

In some embodiments of the present invention, the IFN or IFN mutein is recombinantly produced. As used herein, “recombinantly produced” IFN or IFN mutein of the present invention is IFN or IFN mutein, respectively, that has comparable biological activity to mature, native IFN and that has been prepared by recombinant DNA techniques. The IFN or IFN mutein of the present invention can be produced by culturing a host cell transformed with an expression vector comprising a nucleotide sequence that encodes the IFN or IFN mutein polypeptide. The host cell is one that can transcribe the nucleotide sequence and produce the desired protein, and can be prokaryotic (see, e.g., E. coli) or eukaryotic (e.g., a yeast, insect, or mammalian cell). Examples of recombinant production of IFN or IFN muteins, including suitable expression vectors, are provided in, e.g., Mantei et al. (1982) Nature 297:128; Ohno et al. (1982) Nucleic Acids Res. 10:967; Smith et al. (1983) Mol. Cell. Biol. 3:2156, and U.S. Pat. Nos. 4,462,940, 5,702,699, and 5,814,485; herein incorporated by reference. Also, e.g., see U.S. Pat. No. 5,795,779, where IFNβ is recombinantly produced in Chinese hamster ovary (CHO) cells.

Human IFN genes have been cloned using recombinant DNA (“rDNA”) technology and have been expressed in E. coli (see e.g., Nagola et al. (1980) Nature 284:316; Goeddel et al. (1980) Nature 287:411; Yelverton et al. (1981) Nuc. Acid Res. 9:731; Streuli et al. (1981) Proc. Natl. Acad. Sci. U.S.A. 78:2848). Alternatively, the IFN or IFN muteins of the present invention can be produced, e.g., by a transgenic animal or plant that has been genetically engineered to express the IFN or IFN mutein polypeptide of interest in accordance with methods known in the art.

Proteins or polypeptides that exhibit native IFN or IFN-like properties may also be produced with rDNA technology by extracting poly-A-rich 12S messenger RNA from virally induced human cells, synthesizing double-stranded cDNA using the mRNA as a template, introducing the cDNA into an appropriate cloning vector, transforming suitable microorganisms with the vector, harvesting the microorganisms, and extracting the interferon therefrom (see, e.g., European Patent Application No.s 28033 (published May 6, 1981); 32134 (published Jul. 15, 1981); and 34307 (published Aug. 26, 1981)), which describe various methods for the production of IFNβ employing rDNA techniques.

Alternatively, the IFN or IFN mutein of the present invention can be synthesized chemically, by any of several techniques that are known to those skilled in the peptide art (see e.g., Li et al. (1983) Proc. Natl. Acad. Sci. USA 80:2216-2220, Steward and Young (1984) Solid Phase Peptide Synthesis (Pierce Chemical Company, Rockford, Ill.), and Baraney and Merrifield (1980) The Peptides: Analysis, Synthesis, Biology, ed. Gross and Meinhofer, Vol. 2 (Academic Press, N. Y., 1980), pp. 3-254, discussing solid-phase peptide synthesis techniques; and Bodansky (1984) Principles of Peptide Synthesis (Springer-Verlag, Berlin) and Gross and Meinhofer, eds. (1980), The Peptides. Analysis, Synthesis, Biology, Vol.1 (Academic Press, New York, discussing classical solution synthesis). The IFN or IFN mutein of the present invention can also be chemically prepared by the method of simultaneous multiple peptide synthesis. See, for example, Houghten (1984) Proc. Natl. Acad. Sci. USA 82:5131-5135; and U.S. Pat. No. 4,631,211.

Pharmaceutical Compositions

The pharmaceutical compositions of the present comprise a therapeutically effective amount of an IFN or IFN mutein, and are suitable for use in the methods of the present invention. Examples of suitable IFN include, but are not limited to, IFNα, IFNβ, and IFNγ. In a preferred embodiment, the IFN is an IFNβ (e.g., IFNβ-1a), or an IFNα. Examples of suitable IFN muteins include, but are not limited to, variants of IFNα, IFNβ, or IFNγ. In a preferred embodiment, the IFN mutein is IFNβ-1b (also called IFNβₛₑᵣ₁₇-1b, Betaseron®, Betaferon®).

Methods for formulating pharmaceutical compositions are generally known in the art. For example, see Remington's Pharmaceutical Sciences 18.sup.th ed.: Mack Pub. Co.: Eaton, Pa. 1990, for a thorough discussion on the formulation and selection of pharmaceutically acceptable carriers, stabilizers, and isomolytes. Also, for formulating pharmaceutical compositions comprising IFN or IFN muteins, see, e.g., U.S. Pat. Nos. 4,588,585, 5,183,746; 5,795,779; and 5,814,485; U.S. application Ser. Nos. 10/010,448, 10/190,838, 10/035,397; and PCT International Application No.s PCT/US02/21464 and PCT/US01/51074.

A pharmaceutically acceptable carrier may be used in combination with an IFN or IFN mutein, and other components (e.g., co-medications) in the pharmaceutical compositions of the present invention. As used herein, “pharmaceutically acceptable carrier” is a carrier or diluent that is conventionally used in the art to facilitate the storage, administration, and/or the desired effect of the therapeutic ingredients of the pharmaceutical composition. A carrier may also reduce any undesirable side effects of the therapeutic agent, e.g., an IFN or IFN mutein of the present invention. A suitable carrier is preferably stable, e.g., incapable of reacting with other ingredients in the formulation. Further, a suitable carrier preferably does not produce significant local or systemic adverse effect in recipients at the dosages and concentrations employed for therapy. Such carriers are generally known in the art.

Suitable pharmaceutically acceptable carriers are, e.g., solvents, dispersion media, antibacterial and antifungal agents, microcapsules, liposomes, cationic lipid carriers, isotonic and absorption delaying agents and the like which are not incompatible with the active or therapeutic ingredients (e.g., an IFN or IFN mutein of the present invention) of the pharmaceutical compositions of the present invention. The use of such media and agents for therapeutically effective or active substances is well known in the art. Supplementary active ingredients may also be incorporated into the pharmaceutical compositions of the present invention and used in the methods of the present invention.

Additional examples of pharmaceutically suitable carriers for use in the pharmaceutical compositions of the present invention are large stable macromolecules such as albumin, gelatin, collagen, polysaccharide, monosaccarides, polyvinylpyrrolidone, polylactic acid, polyglycolic acid, polymeric amino acids, fixed oils, ethyl oleate, liposomes, glucose, sucrose, lactose, mannose, dextrose, dextran, cellulose, mannitol, sorbitol, polyethylene glycol (PEG), heparin alginate, and the like. Slow-release carriers, such as hyaluronic acid, may also be suitable.

Stabilizing agents such as human serum albumin (HSA), mannitol, dextrose, trehalose, thioglycerol, and dithiothreitol (DTT), may also be added to the pharmaceutical compositions of the present invention to enhance their stability. Suitable stabilizing agents include but are not limited to ethylenediaminetetracetic acid (EDTA) or one of its salts such as disodium EDTA; polyoxyethylene sorbitol esters e.g., polysorbate 80 (TWEEN 80), polysorbate 20 (TWEEN 20); polyoxypropylene-polyoxyethylene esters e.g., Puronic F68 and Pluronic F127; polyoxethylene alcohols e.g., Brij 35; semethicone; polyethylene glycol e.g., PEG400; lysophosphatidylcholine; and polyoxyethylene-p-t-octyphenol e.g., Triton X-100. Stabilization of pharmaceutical compositions by surfactants is generally known in the art (see e.g., Levine et al. (1991) J. Parenteral Sci. Technol. 45(3):160-165).

Other acceptable components of the pharmaceutical compositions of the present invention may include, but are not limited to, buffers that enhance isotonicity such as water, saline, phosphate, citrate, succinate, acetic acid, aspartate, and other organic acids or their salts. Preferably, pharmaceutical compositions of the present invention comprise a non-ionic tonicifying agent in an amount sufficient to render the compositions isotonic with body fluids. The pharmaceutical compositions of the present invention can be made isotonic with a number of non-ionic tonicity modifying agents generally known to those in the art, e.g., carbohydrates of various classifications (see, e.g., Voet and Voet (1990) Biochemistry (John Wiley & Sons, New York); monosaccharides classified as aldoses (e.g., glucose, mannose, arabinose), and ribose, as well as those classified as ketoses (e.g., fructose, sorbose, and xylulose); disaccharides (e.g., sucrose, maltose, trehalose, and lactose); and alditols (acyclic polyhydroxy alcohols) e.g., glycerol, mannitol, xylitol, and sorbitol. In a preferred embodiment, non-ionic tonicifying agents are trehalose, sucrose, and mannitol, or a combination thereof.

Preferably, the non-ionic tonicifying agent is added in an amount sufficient to render the formulation isotonic with body fluids. In one embodiment, when incorporated into a pharmaceutical composition of the present invention (including, e.g., an HSA-free pharmaceutical composition), the non-ionic tonicifying agent is present at a concentration of about 1% to about 10%, depending upon the agent used (see e.g., U.S. application Ser. Nos. 10/190,838, 10/035,397; and PCT International Application No.s PCT/US02/21464 and PCT/US01/51074).

Other acceptable components of the pharmaceutical compositions of the present invention may include, but are not limited to, e.g., co-medications. Such co-medications are well known in the art and may include, but are not limited to, e.g., those that help alleviate or mitigate adverse effects due to cardiomyopathy and/or endothelial dysfunction e.g., endothelial dysfunction connected to cardiomyopathy, or due to treatment of the disease. Such co-medications include, but are not limited to, e.g., analgesics, non-steroidal anti-inflammatory drugs (NSAIDs), and steroids, as discussed herein and generally known in the art.

Further, preferred pharmaceutical compositions of the present invention may incorporate buffers having reduced local pain and irritation resulting from injection, or improve solubility or stability of a component (e.g., an IFN or IFN mutein) of the pharmaceutical compositions of the present invention. Such buffers include, but are not limited to, e.g., low-phosphate, aspartate, and succinate buffers.

In one embodiment, the pharmaceutical compositions of the present invention comprise a higher, therapeutically effective amount of a pharmaceutical composition that is stabilized and HSA-free. As used herein an “HSA-free” pharmaceutical composition refers to a pharmaceutical composition prepared in the absence of HSA and is thus free of this pharmaceutical excipient. Preferably, the stabilized, HSA-free pharmaceutical compositions of the present invention comprise a higher, therapeutically effective amount of an IFN or IFN mutein that is substantially monomeric and solubilized in a low-ionic-strength formulation. As used herein, “substantially monomeric” IFN or IFN mutein refers to where the majority of an IFN or IFN mutein (by weight) in a composition of the present invention is a monomer and not aggregated e.g., as a dimer, trimer or other multimer. As used herein, “solubilized” IFN or IFN mutein refers to an IFN or IFN mutein of the present invention that is soluble in solution and not precipitated out of solution. Preferably, the low-ionic-strength formulation has an advantage of stabilizing the IFN or IFN mutein of the present invention, and maintaining it in solution in substantially monomeric form. As used herein, a “stabilized” pharmaceutical composition of the present invention (e.g., a stabilized, HSA-free pharmaceutical composition), refers to a pharmaceutical composition of the present invention where the IFN or IFN mutein is substantially monomeric when in solution and is suitable for use in the compositions and methods of the present invention.

As used herein a “low-ionic-strength” formulation is a solution that comprises a concentration of the buffer sufficient to maintain the buffer at low ionic strength, preferably in a range from about 1 mM to about 100 mM. In a preferred embodiment, the low-ionic-strength formulation is a solution having a pH from about 2 to about 5, and an ionic strength from about 1 mM to about 100 mM. Suitable buffers for preparation of the low-ionic-strength formulation include, but are not limited to, e.g., glycine, aspartic acid, glutamic acid, sodium succinate, formate, acetate, citrate, phosphate, histidine, and imidazole. The stabilized and HSA-free compositions and low-ionic-strength formulations of the present invention can be prepared according to methods known in the art (see, e.g., U.S. Pat. Nos. 4,588,585, 5,183,746; 5,795,779; and 5,814,485; U.S. application Ser. Nos. 10/190,838, 10/035,397, 10/821,333; and PCT International Application No.s PCT/US02/21464 and PCT/US01/51074).

The pharmaceutical composition may additionally comprise a solubilizing compound or formulation that is capable of enhancing the solubility of the IFN or IFN mutein of the present invention. Suitable solubilizing compounds include, e.g., compounds containing a guanidinium group, preferably arginine. Additional examples of suitable solubilizing compounds include, but are not limited to, e.g., the amino acid arginine, or amino acid analogues of arginine that retain the ability to enhance the solubility of an IFN or IFN mutein of the present invention. Examples of such amino acid analogues, include but are not limited to, e.g., dipeptides and tripeptides that contain arginine. Further examples of suitable solubilizing compounds are discussed in, e.g., U.S. Pat. Nos. 4,816,440; 4,894,330; 5,005,605; 5,183,746; 5,643,566; and in Wang et al. (1980) J. Parenteral Drug Assoc. 34: 452-462)

In preferred embodiments, the pharmaceutical compositions of the present invention comprise IFN or IFN mutein formulated in a unit dosage and in an injectable form such as a solution, suspension, or emulsion, or in the form of lyophilized powder, which can be converted into solution, suspension, or emulsion prior to administration. The pharmaceutical compositions of the present invention may be sterilized by membrane filtration, which also removes aggregates, and stored in unit-dose or multi-dose containers such as sealed vials, ampules or syringes.

Liquid, lyophilized, or spray-dried pharmaceutical compositions comprising an IFN or IFN mutein may be prepared as known in the art, e.g., as an aqueous or nonaqueous solution or suspension for subsequent administration to a patient in accordance with the methods of the present invention. Each of these pharmaceutical compositions may comprise an IFN or IFN mutein as a therapeutically or prophylactically effective or active component. As used herein, a therapeutically “effective” or “active” component is an IFN or IFN mutein that is included in the pharmaceutical composition of the present invention to bring about a desired therapeutic response with regard to treatment of cardiomyopathy and/or endothelial dysfunction e.g., endothelial dysfunction connected to cardiomyopathy, or condition in a patient having cardiomyopathy and/or endothelial dysfunction e.g., endothelial dysfunction connected to cardiomyopathy, using the pharmaceutical compositions and/or methods of the present invention. Preferably the pharmaceutical compositions of the present invention comprise appropriate stabilizing agents, bulking agents, or both to minimize problems associated with loss of protein stability and biological activity during preparation and storage.

Formulation of the IFN or IFN mutein for use in the pharmaceutical compositions and methods of the present invention are preferably stable under the conditions of manufacture and storage and preserved against the contaminating action of microorganisms such as bacteria and fungi. Methods of preventing microorganism contamination are well known, and can be achieved e.g., through the addition of various antibacterial and antifungal agents.

Suitable forms of the pharmaceutical composition of the present invention may include sterile aqueous solutions or dispersions, and sterile powders for the extemporaneous preparation of sterile injectable solutions or dispersion. Suitable forms are preferably sterile and fluid to the extent that they can easily be taken up and injected via a syringe. Typical carriers may include a solvent or dispersion medium containing, for example, water buffered aqueous solutions (i.e., biocompatible buffers), ethanol, polyols such as glycerol, propylene glycol, polyethylene glycol, suitable mixtures thereof, surfactants, or vegetable oils. Sterilization can be accomplished by any art-recognized technique, including but not limited to filtration or addition of antibacterial or antifungal agents, for example, paraben, chlorobutanol, phenol, sorbic acid or thimerosal. Further, isotonic agents such as sugars or sodium chloride may be incorporated in the subject compositions.

Production of sterile injectable solutions containing an IFN or IFN mutein of the present invention may be accomplished by incorporating the polypeptide in the desired amount, in an appropriate solvent with various ingredients (e.g., those enumerated herein) as desired, and followed by sterilization. To obtain a sterile powder, the above solutions can be vacuum-dried or freeze-dried as necessary.

The IFN or IFN mutein of the present invention can thus be compounded for convenient and effective administration in pharmaceutically effective amounts with a suitable pharmaceutically acceptable carrier in a therapeutically effective dose.

The precise therapeutically effective amount of an IFN or IFN mutein to be used in the compositions and methods of the present invention for application to humans can be determined by the skilled artisan with consideration of individual differences in age, weight, extent of cellular infiltration by inflammatory cells and condition of the patient having cardiomyopathy and/or endothelial dysfunction e.g., endothelial dysfunction connected to cardiomyopathy.

In some embodiments, the therapeutically effective amount of IFN or IFN mutein of the present invention is in a range that is from about 30 mcg to at least about 1000 mcg.

In some embodiments, the therapeutically effective amount of the IFN or IFN mutein of the present invention is in a range from about 30 mcg to about 500 mcg. For example, in some embodiments the therapeutically effective amount is about 30 mcg to about 50 mcg, about 50 mcg to about 75 mcg, about 75 mcg to about 100 mcg, or about 100 mcg to about 125 mcg, about 125 mcg to about 150 mcg, about 150 mcg to about 175 mcg, about 175 mcg to about 200 mcg, about 200 mcg to about 225 mcg, about 225 mcg to about 250 mcg, about 250 mcg to about 275 mcg, about 275 mcg to about 300 mcg, about 300 mcg to about 325 mcg, about 325 mcg to about 350 mcg, about 350 mcg to about 375 mcg, about 375 mcg to about 400 mcg, about 400 mcg to about 425 mcg, about 425 mcg to about 450 mcg, about 450 mcg to about 475 mcg, or about 475 mcg to about 500 mcg. In a preferred embodiment, the therapeutically effective amount is about 30 mcg. In another preferred embodiment, the therapeutically effective amount is about 250 mcg. In another preferred embodiment, the therapeutically effective amount is about 500 mcg.

In some embodiments, the therapeutically effective amount of the IFN or IFN mutein of the present invention is in a range that is at least about 500 mcg to at least about 1000 mcg. For example, in some embodiments, the therapeutically effective amount of IFN or IFN mutein is at least about 500 mcg to at least about 525 mcg, at least about 525 mcg to at least about 550 mcg, at least about 550 mcg to at least about 575 mcg, at least about 575 mcg to about at least about 600 mcg, at least about 600 mcg to at least about 625 mcg, at least about 625 mcg to at least about 650 mcg, at least about 650 mcg to at least about 675 mcg, at least about 675 mcg to at least about 700 mcg, at least about 700 mcg to at least about 725 mcg, at least about 725 mcg to at least about 750 mcg, at least about 750 mcg to at least about 775 mcg, at least about 800 mcg to at least about 825 mcg, at least about 825 mcg to at least about 850 mcg, at least about 850 mcg to at least about 875 mcg, at least about 875 mcg to at least about 900 mcg, at least about 900 mcg to at least about 925 mcg, at least about 925 mcg to at least about 950 mcg, at least about 950 mcg to at least about 975 mcg, or at least about 975 mcg to at least about 1000 mcg.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier.

The principal active ingredients (e.g., an IFN or IFN mutein of the present invention and, optionally, a co-medication) may be compounded for convenient and effective administration in therapeutically effective amounts with a suitable pharmaceutically acceptable carrier in dosage unit form as described herein. A unit dosage form can, for example, contain the principal active compound (i.e., an IFN or IFN mutein of the present invention) in a therapeutically effective amount that is in a range that is from about 30 mcg to at least about 1000 mcg. In some embodiments, the therapeutically effective amount of the IFN or IFN mutein of the present invention is in a range from about 30 mcg to about 500 mcg. In a preferred embodiment, the therapeutically effective amount is about 30 mcg. In another preferred embodiment, the therapeutically effective amount is about 250 mcg. In another preferred embodiment, the therapeutically effective amount is about 500 mcg. In some embodiments, the therapeutically effective amount of the IFN or IFN mutein of the present invention is in a range that is at least about 500 mcg to at least about 1000 mcg.

The co-medications are contained in a unit dosage form in amounts generally known in the art. In the case of compositions containing supplementary active ingredients, e.g. co-medications, the dosages may be determined, e.g., by reference to the known dose and manner of administration of the ingredients.

Packaging material used to contain the active ingredient (i.e., the IFN or IFN mutein) of the pharmaceutical composition of the present invention can comprise glass, plastic, metal or any other suitable inert material and, preferably, is packaging material that does not chemically react with any of the ingredients contained therein.

The pharmaceutical compositions of the present invention may be administered in a manner compatible with the dosage formulation and in such an amount as will be therapeutically effective. Further, the pharmaceutical compositions of the present invention may be administered in any way which is medically acceptable and which may depend on the specific type of cardiomyopathy and/or endothelial dysfunction e.g., endothelial dysfunction connected to cardiomyopathy, or associated symptoms being treated. Possible administration routes include injections, by parenteral routes such as intravascular, intravenous, intra-arterial, subcutaneous, intramuscular, intratumor, intraperitoneal, intraventricular, intraepidural or others, as well as oral, nasal, ophthalmic, rectal, topical, or by inhalation. In a preferred embodiment, the administration route is subcutaneous.

Sustained release administration is also contemplated, e.g., using erodible implants.

In a preferred embodiment, the pharmaceutical composition of the present invention comprises a therapeutically effective amount of an IFN or IFN mutein that is in a range that is from about 30 mcg to at least about 1000 mcg. In some embodiments, the therapeutically effective amount of the IFN or IFN mutein of the present invention is in a range from about 30 mcg to about 500 mcg. In a preferred embodiment, the therapeutically effective amount is about 30 mcg. In another preferred embodiment, the therapeutically effective amount is about 250 mcg. In another preferred embodiment, the therapeutically effective amount is about 500 mcg. In some embodiments, the therapeutically effective amount of the IFN or IFN mutein of the present invention is in a range that is at least about 500 mcg to at least about 1000 mcg. In a preferred embodiment, the IFN is a human IFNβ (e.g., IFNβ-1a), or a human IFNα. In another preferred embodiment, the IFN mutein is a human IFNβ mutein, e.g., IFNβ-1b (also called IFNβ-1bₛₑᵣ₁₇, Betaseron®/Betaferon®), or a human IFNα mutein.

In another preferred embodiment, the IFN or IFN mutein of the present invention is a purified, sterile, lyophilized protein product produced by recombinant DNA techniques and formulated for use by subcutaneous injection. For example, the IFN or IFN mutein can be manufactured by bacterial fermentation of a strain of E. coli that carrys a plasmid encoding the mutein. In a preferred embodiment, the IFN is a human IFNβ (e.g., IFNβ-1a), or a human IFNα. In another preferred embodiment, the IFN mutein is a human IFNβ mutein (e.g., IFNβ-1b), or a human IFNα mutein.

In another preferred embodiment, IFNβ-1b is 165 amino acids in length, has a molecular weight of approximately 18,500 daltons. In another preferred embodiment, the IFNβ-1b polypeptide is made by isolating the human IFNβ gene from human fibroblasts and substituting the serine at position 17 with cysteine. In another preferred embodiment, the specific activity of IFNβ-1b is approximately 32 million international units (IU)/mg.

In a preferred embodiment, IFNβ-1b (also called IFNβ-1bₛₑᵣ₁₇, Betaseron®/Betaferon®) is supplied as a lyophilized powder containing a higher therapeutically effective amount of IFNβ-1b and human albumin USP (United States Pharmacopoeia) and mannitol USP as stabilizers. In one embodiment, the stabilizers are human albumin USP and dextrose USP. In one preferred embodiment, the lyophilized protein product is a sterile, white to off-white powder that is intended for subcutaneous injection after reconstitution with a diluent supplied (e.g., the diluent can be a sodium chloride solution, preferably a 54% solution of sodium chloride).

In a preferred embodiment, the protein product is packaged in a clear glass, single-use vial; and a separate vial containing diluent (e.g., a 0.54% solution of sodium chloride) is included for each vial of drug. In another preferred embodiment, the diluent is provided in a syringe (i.e., the syringe is pre-filled with the diluent). In yet another preferred embodiment, the pharmaceutical composition of the present invention is provided in solution in a syringe (i.e., the syringe is pre-filled with the pharmaceutical composition in solution) and is ready for use.

In a preferred embodiment, the pharmaceutical composition of the present invention can be stored under refrigeration, between 2° to 8° C. (36° to 46° F.). In another embodiment, the pharmaceutical composition is stored at room temperature.

In a preferred embodiment, the pharmaceutical composition of the present invention is administered subcutaneously, every other day. In another preferred embodiment, the subcutaneous administration is via automated or manual injection (e.g., using a syringe) of the pharmaceutical composition.

The invention is further illustrated by the following examples that are not intended in any way to limit the scope of the invention.

### EXAMPLES

### Example 1

### Treatment of Cardiomyopathy using IFNβ-1a

This example describes a phase 11 clinical study that demonstrates the safe and efficacious treatment of chronic viral cardiomyopathy using a therapeutically effective amount of IFNβ-1a.

Summary

Viral infections are important causes of cardiomyopathy and may induce cardiac dysfunction and may finally lead to dilated cardiomyopathy. The purpose of the study was the evaluation of safety and efficacy of recombinant IFNβ-1a (IFNβ-1a) in patients with chronic viral cardiomyopathy. The main efficacy criteria was the elimination of viruses and signs of inflammation from the myocardium measured in myocardial biopsies after IFNβ therapy. Further efficacy criteria were the improvement of myocardial function (e.g., increase of ejection fraction) and of myocardial symptoms such as dyspnea, angina pectoris, palpitations and arrhythmia. In addition, the patients were evaluated according to the New York Heart Association functional classification (heart failure). The results of this demonstrate that IFNβ-1a in the therapy regimen tested is safe and well-tolerated by patients with chronic viral cardiomyopathy, and results in the elimination of Adenovirus and/or Enterovirus from the myocardium in all treated patients and individual improvement in different clinical/hemodynamic parameters.

Background

Some advances in pharmacological therapy have resulted in improved survival in patients with heart failure attributable to dilated cardiomyopathy (DCM) (see e.g., Elliot P. (2000) Eur. Heart J. 84: 106-112). However, current therapy is symptomatic and does not influence specific underlying pathomechanisms. Many DCM patients progress to terminal heart failure, and DCM represents the most common heart failure entity requiring heart transplantation (see e.g., Elliot P. (2000) Eur. Heart J. 84: 106-112; Feldmann et al. (2000) N. Engl. J. Med. 343:1388-1398; Richardson et al. (1996) Circulation 93: 841-842). Enteroviruses and adenoviruses are the most frequently implicated pathogens in Western countries (see e.g., Bowles et al. (1986) Lancet 1: 1120-1123; Tracy et al. (1990) J. Am. Coll. Cardiol. 15:1688-1694; Jin et al. (1990) Circulation 82:8-16; Archard et al. (1991) Eur. Heart J. 12:56-59; Koide et al. (1992) Jpn. Circ. J. 56: 1081-1093; Grasso et al. (1992) Am. J. Cardiol. 69: 658-664; Baboonian C. (1997) Heart 78: 539-543; Pauschinger et al. (1999) Circulation 99: 889-895).

The detection of enteroviral genomes in the myocardium is associated with an adverse prognosis and an independent predictor of clinical outcome (see e.g., Why et al. (1994) Circulation 89: 2582-2589; Fujioka et al. (2000) J. Am. Coll. Cardiol. 36: 1920-1926). These observations led to a search for a specific antiviral therapy in this subgroup of patients with DCM. The antiviral potential of interferon IFNβ against coxsackieviruses has been demonstrated in vitro14 and in animal models (see e.g., Matsumori (1988) Am. Heart J. 115: 1229-1232). However, the effect of IFNs against adenoviral infection has not yet been elucidated.

The present study was undertaken to investigate whether IFNβ-1a treatment of patients with biopsy-proven myocardial persistence of enteroviral or adenoviral genomes is safe and if it may achieve elimination of viral genomes, which could possibly prevent progression of cardiac dysfunction. To include patients expected likely to benefit from antiviral therapy, two inclusion criteria were used (virus persistence and long-term cardiac dysfunction despite conventional heart failure therapy) reported to be associated with an adverse prognosis (see e.g., Why et al. (1994) Circulation 89: 2582-2589; Fujioka et al. (2000) J. Am. Coll. Cardiol. 36: 1920-1926).

Methods and Study Design

In this phase 11 study, 32 consecutive patients with chronic viral cardiomyopathy were treated with 6×10⁶ IU IFNβ-1a by injection thrice weekly subcutaneously for 24 weeks.

As safety criteria, the specific cardiac adverse events as well as laboratory values and other adverse events were documented.

Diagnosis and Main Criteria for Inclusion
Virus persistence detected in endomyocardial biopsy with/or without signs of myocardial inflammation
no improvement of hemodynamic parameters within the last 6 months during constant basic therapy
reduced global or regional left ventricular contractility, left ventricular ejection fraction (LVEF)>25% or
lack of myocardial pumping reserve on exertion and normal LVEF at rest, determined by radionuclide ventriculography (RNV) or
symptomatic arrhythmia of unclear etiology
written informed consent

Criteria for Evaluation

Efficacy

Main criteria for the evaluation of efficacy were the elimination of viruses, proven by nested polymerase chain reaction (n-PCR) after the end of the 24 weeks therapy, as well as the disappearance of histologically proven signs of inflammation.

In addition, in patients with an enlarged left ventricle prior to therapy, improvement was assessed by decreased left ventricular enddiastolic and endsystolic diameters. The LVEF at rest was evaluated by radionuclide ventriculography prior to and after therapy. Improvements of cardiac symptoms such as dyspnea, angina pectoris, palpitations and arrhythmia were evaluated by the investigator through questioning the patients. Patients were also assessed according to the classification of the New York Heart Association (NYHA functional class).

Safety

Safety was evaluated through questioning of the patients by the investigator and the reported adverse events were classified into the categories “mild”, “moderate” or “severe The investigator had to determine if there was a relationship to the study medication. The relationship to the study medication had to be classified as “no”, “unlikely”, “possible”, “probable”, “definite” or “unknown”.

Study Patients

Demographic and Baseline Characteristics

In total, 32 patients were enrolled into the study. The patients were recruited between 17 Apr. 1998 and 23 Nov. 2000. The treatment of the last recruited patient was completed on 7 May 2001. The first 22 study patients (characterized as LY) received a human albumin-containing IFNβ-1a formulation, the following 10 patients a human albumin-free IFNβ-1a formulation (characterized as FL). Twelve female and 20 male patients, aged on average 50 years, participated in the study. All patients were Caucasians, the median body mass index was nearly 27.

All patients had a viral infection of the myocardium proven by nested PCR in the endomyocardial biopsies. At study entry the myocardial biopsy was positive for Enterovirus in 20 patients and for Adenovirus in 11 patients. Two of these patients were positive for both. Five patients, all in the FL group, were positive for Parvovirus. Two out of these five patients were additionally positive for Adenovirus or Enterovirus. Positive virus detection in the myocardial biopsy was the main inclusion criterion and virus elimination was the primary outcome variable. Positive results of Parvovirus were detected from 4 FL patients after therapy but no data from baseline exists for these patients. Positive results of HHV6 were detected in 3 FL patients after therapy but no data from baseline exists for these patients.

The LVEF at rest, measured by RNV showed a median figure of 44% at study start, ranging from 19% to 71%. The median LVEF during exercise was 42%, ranging from 15% to 77%. The individual differences of the LVEF at rest and during exercise were calculated and evaluated. The median LVEF was increased at maximum workload by 1 percentage point (and the mean by 1.5 points). Regional wall motion abnormalities were found in 12 patients (37%), but for another 6 patients (19%) this information was missing.

All patients have had a common heart failure therapy at baseline. Twenty-four patients received beta-blockers, 23 ACE inhibitors, 19 digitalis, 15 diuretics, 4 antiarrhythmic therapy and 2 calcium channel antagonists.

Overall Study Design

The present study was performed as a monocenter, open and uncontrolled clinical phase 11 study in patients with chronic viral cardiomyopathy. The originally planned treatment was IFNβ-1a in a dose of 6 MIU subcutaneously 3 times weekly for 24 weeks. In the first week the patients received only 2 MIU per application and in the second week 4 MIU per application.

Following the first Protocol Amendment it was left to the discretion of the investigator whether or not to extend the therapy for up to a further 6 months. After enrollment of 22 patients a new human serum albumin (HSA) free liquid formulation became available and, following a second Protocol Amendment, this new formulation was administered to a further 10 patients. A total number of 32 patients to be enrolled in the study was determined to be adequate to obtain first safety and efficacy data in this indication and with this new therapy approach. Only those patients who signed informed consent were enrolled in the study. Each patient had to satisfy all the inclusion and exclusion criteria.

The study consisted of the following study periods or check points, respectively:

week 0: selection of patients according to inclusion and exclusion criteria.

week 1: 3×2 MIU IFNβ-1a, (LY) or 3×1 MIU (FL).

week 2: 3×4 MIU IFNβ-1a (LY) or 3×3 MIU (FL).

weeks 3-24: 3×6 MIU IFNβ-1a (LY or FL).

For three patients treatment was extended (for 11 to 13 weeks) followed by a 6 month treatment-free follow up as performed in the other patients. week 25-48: Follow up.

A summary of the efficacy and safety measurements performed during the study is given in the following Table 1, below.

TABLE 1
Summary of the efficacy and safety measurements performed during the study

| | Screening | Treatment | | | | | | | | | FU |
|---|---|---|---|---|---|---|---|---|---|---|---|
| | 0 | 1 | 2 | 3 | 4 | 5-8 | 9-12 | 13-16 | 17-20 | 21-24 | 25-48* 48-96 |
| Visit (code) | 0 | 1 | 2 | 3 | 4 | 5 | 6 | 7 | 8 | 9 | 60-65 |
| Informed consent | • | | | | | | | | | | |
| Demographic data | • | | | | | | | | | | |
| Medical history | • | | | | | | | | | | |
| Inclusion/ | • | | | | | | | | | | |
| Exclusion criteria | | | | | | | | | | | |
| Laboratory I | • | • | • | • | • | • | • | • | • | • | • |
| (Hematology; | | | | | | | | | | | |
| Chemistry) | | | | | | | | | | | |
| Laboratory II | • | | | | | | | | | • | |
| (Virus serology) | | | | | | | | | | | |
| Physical | • | • | • | • | • | • | • | • | • | • | • |
| examination | | | | | | | | | | | |
| Concomitant | • | | | | | | | | | | |
| diseases | | | | | | | | | | | |
| Concomitant | • | • | • | • | • | • | • | • | • | • | • |
| medication | | | | | | | | | | | |
| Electrocardiography | • | • | • | • | • | • | • | • | • | • | • |
| (ECG) | | | | | | | | | | | |
| Echocardiography | • | • | • | • | • | • | • | • | • | • | • |
| Radionuclide ventriculography | • | | | | | | | | | | • |
| (RNV) | | | | | | | | | | | |
| Chest X-ray | • | | | | | | | | | | • |
| Heart | • | | | | | | | | | | • |
| catheterization | | | | | | | | | | | |
| Immunohistochemistry | • | | | | | | | | | | • |
| Histology | • | | | | | | | | | | • |
| PCR-virus | • | | | | | | | | | | • |
| detection | | | | | | | | | | | |
| Therapy | | Study treatment according to the study protocol | | | | | | | | | |
| Adverse events | | • | • | • | • | • | • | • | • | • | • |
| NYHA functional | • | | | | | | | | | | • |
| class** | | | | | | | | | | | |
| Symptoms | • | • | • | • | • | • | • | • | • | | • |
| (Dyspnea, | | | | | | | | | | | |
| Angina, Palpitations)** | | | | | | | | | | | |

*In 3 patients the study treatment was extended by 10-13 weeks after the end of the regular study treatment. This treatment phase was coded with the visit 30-32.
**Evaluation not defined in the protocol.

Follow up visits and the visits in the additional treatment phase (coded 60-65 or 30-32) were not scheduled for a specific time or number. The occurrence of adverse events was evaluated by asking the patients at each visit it they had had any adverse events since the last visit and by assessment of laboratory findings.

Results

Efficacy

With respect to the main efficacy parameter (i.e., the elimination of viruses): All patients with the presence of Adenovirus (n=11) and/or Enterovirus (n=20) before treatment with IFNβ-1a showed, in the myocardial biopsy, an elimination of these viruses after therapy (see Table 2, below). For one patient there was no data available after therapy because the patient had withdrawn informed consent for the heart catheter examination and for the taking of a biopsy. Three out of 5 patients with presence of Parvovirus at baseline kept their viruses after therapy. In one patient the virus was eliminated and from one patient no biopsy after therapy was available. The assessment of the efficacy parameter “elimination of signs of inflammation” was difficult since only one patient showed a borderline myocarditis and 3 patients had “remarkable findings” prior to start of therapy. Only one patient showed remarkable signs of inflammation after therapy.

TABLE 2
Detection of Adenovirus, Enterovirus and Parvovirus at before and after therapy

| | Screening | | | After therapy | | |
|---|---|---|---|---|---|---|
| Patient | Adenovirus | Enterovirus | Parvovirus | Adenovirus | Enterovirus | Parvovirus |
| FL1 | − | − | + | missing | missing value | Missing |
| | | | | value | | value |
| FL2 | − | + | missing | − | − | + |
| | | | value | | | |
| FL3 | − | + | missing | − | − | + |
| | | | value | | | |
| FL4 | + | − | missing | − | − | + |
| | | | value | | | |
| FL5 | − | + | missing | − | − | − |
| | | | value | | | |
| FL6 | − | + | missing | − | − | + |
| | | | value | | | |
| FL7 | + | − | + | − | − | + |
| FL8 | − | − | + | − | − | + |
| FL9 | − | + | + | − | − | − |
| FL10 | − | − | + | − | − | + |
| LY1 | − | + | − | − | − | Missing |
| | | | | | | value |
| LY2 | missing | + | − | − | − | Missing |
| | value | | | | | value |
| LY3 | − | + | − | − | − | Missing |
| | | | | | | value |
| LY4 | − | + | − | − | − | Missing |
| | | | | | | value |
| LY5 | − | + | − | missing | missing value | Missing |
| | | | | value | | value |
| LY6 | + | + | − | − | − | Missing |
| | | | | | | value |
| LY7 | missing | + | − | missing | − | Missing |
| | value | | | value | | value |
| LY8 | − | + | − | − | − | Missing |
| | | | | | | value |
| LY9 | + | − | − | − | − | Missing |
| | | | | | | value |
| LY10 | + | + | − | − | − | Missing |
| | | | | | | value |
| LY11 | missing | + | − | − | − | Missing |
| | value | | | | | value |
| LY12 | + | − | − | − | − | Missing |
| | | | | | | value |
| LY13 | − | + | missing | − | − | Missing |
| | | | value | | | value |
| LY14 | missing | + | − | − | − | Missing |
| | value | | | | | value |
| LY15 | − | + | missing | − | − | Missing |
| | | | value | | | value |
| LY16 | − | + | − | missing | − | Missing |
| | | | | value | | value |
| LY17 | + | − | − | − | − | Missing |
| | | | | | | value |
| LY18 | + | − | − | − | − | Missing |
| | | | | | | value |
| LY20 | + | − | − | − | − | Missing |
| | | | | | | value |
| LY21 | + | − | − | − | − | Missing |
| | | | | | | value |
| LY22 | − | + | missing | − | − | Missing |
| | | | value | | | value |

− = negative;
+ = positive

Taking together all the data on the histology and immunohistology was detected at the start of the study there were no patients with active myocarditis, one patient with borderline myocarditis, 3 with remarkable findings and 24 with no remarkable findings. In 4 cases no data related to the inflammation of the myocardium is available. After therapy there is one patient with remarkable findings, 27 patients with no remarkable findings and 4 missing values.

The secondary efficacy parameter “improvement of myocardial function”, measured by change of LVEF (RNV) at rest, showed an increased median from 44% before therapy to 47% after therapy and an increased mean from 44.3% to 48.3%.

The improvement of myocardial symptoms was measured using the NYHA classification system and the single symptoms dyspnea, angina at rest and on exertion, and palpitations.

Prior to therapy 2 patients were classified as NYHA functional-class I, 12 patients as class II, 11 patients as class IIIA and 7 patients as class IIIB. In 1 patient NYHA functional class was not assessed. After therapy with IFNβ-1a, 13 patients were classified as NYHA-class I, 12 patients as class II, 3 patients as class IIIA and 2 patients as class IIIB. In 2 patients NYHA functional class was not assessed. Twenty-one out of 28 evaluable patients showed an improvement regarding the NYHA functional class after the end of study treatment compared to baseline. No patient showed a deterioration.

Prior to the treatment only 5 patients had no dyspnea. In 12 patients this symptom disappeared during the study. Nine patients suffered from angina pectoris at rest prior to therapy and only 4 patients after therapy. Angina pectoris on exertion was reported in 5 patients prior to treatment and only in 2 patients after therapy. Fifteen patients suffered from palpitations prior to therapy and 8 patients after the treatment. No restriction of personal well being was reported for 7 patients prior to therapy and for 15 patients after the study.

Safety

Overall, 406 adverse events (AEs) were reported. Among these 406 AEs 5 were classified as severe, 42 as moderate and 358 AEs were classified as mild (classification of one AE is missing). For 337 AEs (83%) no action was taken and for 6 AEs (in one patient) study treatment was discontinued. For 5 AEs treatment was interrupted. For 98 AEs the treating physician classified a relationship of the drug to the AE as “definite”, for 94 AEs as “probably related”. For 3 AEs the relationship was not assessed for one AE the relationship is unknown.

The most frequent adverse events were injection site reactions, fatigue, arthralgia, dyspnea, headache, influenza-like symptoms, angina pectoris and dizziness.

Biopsy Results

After 24 weeks of IFNβ-1a treatment, neither enteroviral nor adenoviral genomes were traceable in any biopsy specimens by nested PCR. According to the histologic Dallas criteria, none of the endomyocardial biopsies was graded as active or borderline myocarditis, neither before nor after the IFNβ-1a treatment. In the immunohistochemical analysis, 7 patients had an increased number of CD3-positive T-lymphocytes quantitated at 19.2±4.8 cells/mm2, consistent with myocardial inflammation (see e.g., Kuhl et al. (1996) Heart 75: 295-300). After IFNβ-1a treatment, T-lymphocyte counts had decreased to 6.0±3.1 cells/mm². Lymphocyte numbers in the 15 patients without myocardial inflammation (baseline) did not change significantly.

Discussion

Myocardial Virus Persistence in Dilated Cardiomyopathy

Viral infections have been associated with the development of myocarditis (viral-associated inflammatory cardiomyopathy) for many years (see e.g., Woodruff et al. (1980) Am. J. Pathol. 101: 425-484; Feldman et al. (2000) N. Engl. J. Med. 343: 1388-1398). A correlation of special cardiomyopathy with the development of dilated cardiomyopathy has been reported (see e.g., Kawaii et al. (1999) Circulation 99: 1091 -1100; Liu et al. (2001) 104: 1076-1082). Using molecular genetic methods, enteroviral genomes have been detected in 10% to 35% of endomyocardial biopsies of patients with DCM (see e.g., Bowles et al. (1986) Lancet 1: 1120-1123; Tracy et al. (1990) J. Am. Coll. Cardiol. 15:1688-1694; Jin et al. (1990) Circulation 82:8-16; Archard et al. (1991) Eur. Heart J. 12:56-59; Koide et al. (1992) Jpn. Circ. J. 56: 1081-1093; Grasso et al. (1992) Am. J. Cardiol. 69: 658-664; Baboonian C. (1997) Heart 78: 539-543; Pauschinger et al. (1999) Circulation 99: 889-895; Archard et al. (1988) Hum. Pathol. 29: 578-584). Furthermore, adenoviruses emerged as relevant infectious pathogens in DCM (see e.g., Pauschinger et a. (1999) Circulation 99:1348-1354).

During the natural course of viral heart disease, an early local and systemic immune response limits uncontrolled virus spreading and usually achieves virus elimination from the target organ. However, viruses may escape immune surveillance and establish persistent myocardial infections. The causes for the development of virus persistence and the mechanisms by which viruses cause progression of myocardial dysfunction are incompletely understood.

Natural Course and Molecular Pathogenesis of the Disease

With respect to the natural course of the disease, the present inventors have found that enteroviral nucleid acid persistence is associated with progression of LV dysfunction and lack of clinical improvement (for details, see the Data Supplement). Furthermore, the presence of enteroviral RNA in 34% of 120 consecutively studied patients with heart muscle disease has been reported (see e.g., Why et al. (1994) Circulation 89: 2582-2589). At follow-up, the virus-positive patients had an increased mortality compared with virus-negative patients.

Concerning the underlying pathomechanism, it has been reported that myocardial enteroviral genome persistence and restricted viral replication is sufficient for the maintenance of chronic inflammation, structural alterations of the myocardium, and interference with cardiomyocyte function in animal models of coxsackievirus B3-infected mice (see e.g., Wessely et al. (1998) Circulation 98: 450-457). The prognostic significance of enteroviral infection and replication has also been reported in humans (see e.g., Why et al. (1994) Circulation 89: 2582-2589; Fujioka et al. (2000) J. Am. Coll. Cardiol. 36: 1920-1926).

By using strand-specific PCR analyses, the present inventors have demonstrated that enteroviral genomes may actively replicate in the myocardium of patients with dilated cardiomyopathy, suggesting that enteroviruses may exert an ongoing myocytopathic effect in patients with chronic viral persistence. This observation is consistent with the reporting of the importance of active viral RNA replication as a prognostic marker for poor clinical outcome (see e.g., Fujioka et a. (2000) J. Am. Coll. Cardiol. 36: 1920-1926). Moreover, it has been reported that most patients not responding to immunosuppressive therapy were enterovirus positive in a retrospective PCR study, demonstrating virus-induced myocyte damage during immunosuppressive treatment (see e.g., Frustaci et al. (2003) Circulation. 107: 857-863).

Because enteroviruses may actively replicate even in late stages of infection (see e.g., Pauschinger et a. (1999) Circulation 99: 889-895; Kawai C. (1999) Circulation 99:1091 -1100; Klingel (1998) et al. Lab Invest. 78: 1227-1237), direct myocytopathic effects could perpetuate LV dysfunction in patients with virus persistence, e.g., by interference with cardiomyocyte energy delivery and transmission, matrix integrity, or inflammatory cytokine release (see e.g., Badorff et al. (1999) Nat Med. 5: 320-326; Doerner et al. (2000) J. Am. Coll. Cardiol. 35: 1778-1784; Badorff et al. (2000) Circulation 102: 2276-2281).

IFNβ1-a Therapy in Chronic Viral Cardiomyopathy (CVC)

The objectives of the study were the evaluation of efficacy and tolerability of IFNβ-1a in patients with chronic viral cardiomyopathy. The results of the present study represent uncontrolled data regarding tolerability and efficacy of an IFNβ-1a therapy in patients with CVC.

Concerning the primary efficacy parameter, i.e., the elimination of virus in myocardial biopsies, all patients with Adenovirus and/or Enterovirus prior to therapy eliminated these by the end of the IFNβ-1a therapy. Even though there was no control group in this trial, an elimination rate of these viruses of 100% indicates a clear efficacy with reference to this parameter. A spontaneous elimination of these viruses with such a high elimination rate is extremely improbable.

Further efficacy parameters such as improvement of LVEF, improvement of cardiac symptoms such as dyspnea, angina pectoris and palpitations, or a decrease of left ventricular endsystolic or enddiastolic diameters in patients with enlarged diameters, may be dependent on virus elimination or may reflect a spectrum of repsonses induced by IFNβ. A tendency towards an improvement in all these parameters could be shown Further, in individual patients the course of these parameters showed an impressive improvement. For the parameter improvement in the NYHA classification system, it could be shown that 15 out of 21 evaluable patients with NYHA II to NYHA IIIB improved and no patient suffered from a deterioration.

The tolerability of this treatment could be assessed as very good since only one patient discontinued therapy with IFNβ-1a due to adverse events (moderate flu-like symptoms) and only one patient had a serious adverse event (arrhythmia) with a possible relationship to the treatment. In addition, more than 98% of all reported adverse events were classified as mild (88%) or moderate (10%). The assessment of laboratory values in hematology and blood chemistry indicates that there were only a small number of values outside the normal range. These values outside the normal range did not necessitate any treatment modification.

The partial reversibility of cardiac dysfunction in these patients additionally suggests that the progression of LV dysfunction in patients with virus persistence is not solely caused by an irreversible loss of cardiomyocytes but may in part be attributable to interference of virus-encoded proteins or mRNAs with cardiomyocyte function and matrix integrity (see e.g., Badorff et al. (1999) Nat Med. 5: 320-326; Doerner et al. (2000) J. Am. Coll. Cardiol. 35: 1778-1784). In addition, virus-induced negative inotropic cytokines may contribute to the hemodynamic deterioration (see e.g., Bozkurt et al. (1998) Circulation 97: 1382-1391).

Conclusion

In summary, IFNβ-1a in the therapy regimen tested is safe and well tolerated by patients with chronic viral cardiomyopathy and demonstrates the elimination of Adenovirus and/or Enterovirus from the myocardium in all treated patients, as well as the individual improvement in different clinical/hemodynamic parameters.

### Example 2

### Treatment of Cardiomyopathy using IFNβ-1b

This example describes the preliminary interim results of a study indicating the safe and efficacious treatment of chronic viral cardiomyopathy using a therapeutically effective dose of IFNβ-1b.

Summary

This study evaluates the efficacy and safety of two doses of Betaferon® (IFNβ-1b) in patients with biopsy-diagnosed viral cardiomyopathy (Parvovirus, or Human Herpes Virus 6 which is also termed HHV6). The safe and efficacious use of IFNβ-1b (4 and 8 MIU) in the treatment of patients with chronic viral cardiomyopathy (e.g., Parvovirus and HHV6) can be evaluated and carried out as described herein.

More particularly, this study, including the treatment and follow-up schedule as well as the efficacy and safety parameters can be determined and carried out as described herein including e.g., as described in Examples 1 (above) and Example 3 (below). The interim results from 32 patients of are described herein. The interim analysis of these first 32 patients of this study shows similar efficacy results compared to the 29 patients with Enterovirus/Adenovirus of the study described in Example 2 (above) investigating treatment with IFNβ-1a in patients. It is still to be clarified whether Parvovirus B19 and/or HHV6 induce chronic cardiomyopathy or whether these viruses potentiate an existent cardiomyopathy via reactive mechamisms.

Methods and Study Design (Table 3)

Table 3 shows methods and study design for an open-label, two-arm, single-center study to evaluate efficacy and safety of Betaferon® (IFN-1b) given subcutaneously every other day over 24 weeks in patients with chronic viral cardiomyopathy (parvovirus and HHV6). These patients have chronic viral cardiomyopathy with or without virus-associated inflammatory cardiomyopathy.

TABLE 3
Study outline

| Study phase | Phase II |
|---|---|
| Investigational product, | Betaferon ® (IFNβ-1b), sterile vials with lyophilisate and vials with |
| dosage, | diluent; subcutaneous (s.c.) application every other day: 4 million |
| and route of administration | international units (MIU) or 8 MIU per application over 24 weeks |
| Indication | Chronic viral cardiomyopathy (parvovirus and HHV6) |
| Study objectives | Evaluation of efficacy and safety; efficacy variables: absence of |
| | parvovirus and HHV6 in endomyocardial biopsies and improvement in |
| | New York Heart Association (NYHA) classification; the improvement |
| | must cover at least one NYHA class; presence of parvovirus and/or |
| | HHV6; NYHA class; 6-minute walking-test; single clinical symptoms |
| | (fatigue, dyspnea, angina at rest/on exertion, palpitation, quality of life |
| | (QoL), left ventricular (LV) ejection fraction (EF) at rest/on exertion, LV |
| | function and dimensions, wedge pressure and pulmonary artery |
| | pressure during exercise and rest, inflammatory state in |
| | endomyocardial biopsies; mortality; hospitalization for heart failure |
| Patient population | Patients aged 18 to 75 with biopsy-proven CVC, showing impaired LV |
| | function as evaluated by RNV (30% ≦ EF ≦ 55%) or impaired LV |
| | function as evaluated by RNV (25% ≦ EF < 30%) and wedge pressure |
| | <20 mm Hg or typical symptoms and signs of heart failure and |
| | preserved left ventricular systolic function and no valvular |
| | abnormalities on echocardiography and NYHA II to III; onset of clinical |
| | symptoms at least 6 months prior to enrollment in the study, no |
| | relevant changes of medication of chronic heart failure within 6 |
| | months prior to enrollment in the study as documented by medical |
| | records. |
| Study design | Open-label, two-arms |
| Duration of treatment | 24 weeks |
| Methodology | Determination of virus in endomyocardial biopsies by polymerase |
| | chain reaction (PCR); determination of NYHA class by means of a |
| | questionnaire; determination of single clinical symptoms, |
| | determination of angina according to the Canadian Cardiovascular |
| | Society Classification System, Minnesota Living with Heart Failure |
| | Questionnaire; radionuclide ventriculography (RNV); |
| | echocardiography; histological and immunohistochemical techniques |
| Number of study centers | 1 |
| Total number of patients, | 32 (16 per group) to be randomized |
| minimum and maximum | |
| number of patients per | |
| center, statistical rationale | |
| provided | |
| Adverse events | As reported spontaneously by the patient and as evaluated by |
| | laboratory measurements, physical examinations, echocardiography, |
| | and electrocardiography. |
| Plan for data analysis | Descriptive analysis of parameters |

Table 4 summarizes measurements performed during the study.

TABLE 4
Summary of the efficacy and safety measurements performed during the study

| | | | | | | | | | | |
|---|---|---|---|---|---|---|---|---|---|---|
| Visit | BL | T0 | T1 | T2 | T3 | T4 | T5 | T6 | FU1 | FU2 |
| Week | −1 | 0 | 2 | 4 | 8 | 12 | 16 | 24 | 12 | 24 |
| Deviation Range in ± Days | 2 | − | 2 | 2 | 7 | 7 | 7 | 14 | 14 | 14 |
| Informed consent | • | | | | | | | | | |
| Study incl./excl. criteria | • | | | | | | | | | |
| Randomization | • | | | | | | | | | |
| Demographic data | • | | | | | | | | | |
| Medical and surgical history | • | | | | | | | | | |
| Vital signs | • | | • | • | • | • | • | • | • | • |
| Physical examination | • | | • | • | • | • | • | • | • | • |
| Laboratory A | • | | • | • | • | • | • | • | • | • |
| Laboratory B | • | | | | | | | • | | • |
| NYHA | • | | | | | | | | • | • |
| IFNβ neutralizing antibodies (NABs) | [symbol] | | | | | | [symbol] *** | [symbol] *** | [symbol] *** | [symbol] |
| RNV** | • | | | | | | | | • | |
| Evaluation of single clinical | • | | | | | | | | • | • |
| QoL | • | | | | | | | | • | • |
| Patient global assessment | | | | | | | | | • | • |
| Echocardiography | • | | • | • | • | • | • | • | • | • |
| ECG | • | | • | • | • | • | • | • | • | • |
| 1^{st} application of study medication | | • | | | | | | | | |
| Study medication dispense | | • | | | • | | • | | | |
| Study medication return | | | • | • | • | • | • | • | • | |
| Coagulation* | • | | | | | | | | • | |
| Right heart catheterization and | | | | | | | | | • | |
| WP and PAP at rest and during | • | | | | | | | | • | |

| | |
|---|---|
| Adverse events | continuously |
| Previous and concomitant | continuously |

BL = Baseline;
FU = follow-up;
SCR = Screening
• = evaluation at study site; [symbol]
= central analysis.;
S1, S2 Screening visits;
T0-T6 Treatment visits.
*to be determined prior to heart catheterization.
**only if not determined prior to the study
***the analysis of these samples will only be performed if a positive result is obtained at FU week 24.

1. Classification and Questionnaire for Evaluation of NYHA Functional Class

The system most commonly used to assess clinical status in patients with heart failure is the classification systems of the New York Heart Association. Since this classification system covers the symptoms most frequently reported in patients with chronic viral cardiomyopathy, it will be used for an overall assessment of symptoms. For reasons of standardization, the assessment will be performed by means of a questionnaire (see below).

| | |
|---|---|
| NYHA I | Patients with cardiac disease without concomitant limitations of physical activity. |
| | Ordinary physical activity does not cause undue fatigue, palpitation, dyspnea, or |
| | anginal pain. |
| NYHA II | Patients with cardiac disease that results in slight limitation of physical activity. |
| | They are comfortable at rest. Ordinary physical activity results in fatigue, |
| | palpitation, dyspnea, or anginal pain. |
| NYHA III | Patients with cardiac disease that results in marked limitation of physical activity. |
| | They are comfortable at rest. Less than ordinary physical activity causes fatigue, |
| | palpitation, dyspnea, or anginal pain. |
| NYHA IV | Patients with cardiac disease that results in inability to carry on any physical |
| | activity without discomfort. Symptoms of cardiac insufficiency or of the anginal |
| | syndrome may be present even at rest. If any physical activity is undertaken, |
| | discomfort is increased. |

Does the patient have symptoms (dyspnea, fatigue, palpitation, angina pectoris) most likely related to cardiac disease which occur and limit the physical activity?
NYHA IV
1. At rest?
2. When getting up, standing, sitting, eating, lying relaxed or dressing?
3. When washing, brushing teeth or having a shower?
4. When walking around in the house?
   NYHA III .
5. When having sexual intercourse?
6. When roller-skating, dancing foxtrot, gardening, raking or weeding?
7. When slowly climbing up to 2 flights of stairs?
8. When walking on level ground at 4 miles per hour?
   NYHA II
9. When carrying heavy objects (80 pounds), shoveling snow or soil, skiing, playing squash, football or handball?
10. When climbing 2 or more flights of stairs at normal speed?
11. When walking or jogging at 5 miles per hour?
   NYHA I
12. When performing vigorous activity?
   Of which kind are the symptoms?

2. Composite Clinical Endpoint

The composite clinical endpoint and patient global assessment used in this study was assessed similarly to the description by Packer, M (2001) Journal of Cardiac Failure; 7:176-182. The composite clinical endpoint will be evaluated as secondary efficacy variable. It includes the assessment of NYHA functional class, patient's global assessment, and major clinical adverse events.

The composite clinical end point will be evaluated by the investigator as follows:
improved: favorable change in NYHA functional class and/or patient global assessment, and no major adverse clinical events
worsened: worsening in NYHA functional class or patient global assessment, or major adverse clinical events
unchanged: neither improved nor worsened
   Patient Global Assessment
marked improvement
moderate improvement
mild improvement
no change
slight deterioration
moderate deterioration
marked deterioration
   The patient global assessment will be assessed by the patient him-/herself and will be documented in the CRF by the independent NYHA physician. If the patient global assessment and the NYHA class go into opposite directions the NYHA assessment should be checked.
   Major Clinical Adverse Events:
death
hospitalization for heart failure with a minimum of 24 hours and requiring an intervention for heart failure (e.g. use of intravenous medication for heart failure)
transplantation
life-threatening arrhythmias
myocardial infarction
pulmonary embolism
stroke
decompensation of chronic left heart failure
cardiogenic pulmonary edema
cardiogenic pre-shock and cardiogenic shock
implantation of left or biventricular assist device and surgical procedures for heart failure other than transplantation (e.g. cardiomyoplasty, partial left ventriculectomy)

3. Other Parameters

Radionuclide ventriculography (left ventricular ejection fraction), hemodynamic measurement (wedge pressure, mean pulmonary artery pressure) and echocardiography (regional and global wall motion, left ventricular diameters) are performed as frequently used measurements in order to assess impairment of systolic and/or diastolic cardiac function.

Results

Study Population and Study Treatment

The demographic data of the 32 patients are shown in table 5. Thirty-one patients completed treatment with IFNβ-1b whereas one patient did not complete treatment with the study medication. The duration of study treatment is shown in table 6.

TABLE 5
Age, height and body weight at baseline by gender and overall - All
randomized patients

| | | | | | | | | |
|---|---|---|---|---|---|---|---|---|
| | Demo- | | | | | | | |
| | graphic | | | | | | | |
| Gender | variable | N | Nmiss | Mean | SD | Min | Median | Max |

4 MIU IFNβ-1b

| | | | | | | | | |
|---|---|---|---|---|---|---|---|---|
| female | Age | 7 | 0 | 8.76 | 11.82 | 29.7 | 48.39 | 62.6 |
| | [years] | | | | | | | |
| | Weight | 5 | 2 | 70.60 | 11.72 | 51.0 | 73.00 | 80.0 |
| | [kg] | | | | | | | |
| | Height | 5 | 2 | 171.60 | 3.21 | 168.0 | 170.00 | 175.0 |
| | [cm] | | | | | | | |
| | BMI | 5 | 2 | 23.92 | 3.61 | 18.1 | 24.22 | 27.7 |
| | [kg/m²] | | | | | | | |
| male | Age | 7 | 0 | 58.90 | 11.67 | 38.2 | 60.70 | 76.2 |
| | [years] | | | | | | | |
| | Weight | 4 | 3 | 95.40 | 9.61 | 90.0 | 90.90 | 109.8 |
| | [kg] | | | | | | | |
| | Height | 5 | 2 | 177.60 | 7.73 | 167.0 | 176.00 | 187.0 |
| | [cm] | | | | | | | |
| | BMI | 3 | 4 | 31.85 | 6.63 | 26.9 | 29.31 | 39.4 |
| | [kg/m²] | | | | | | | |

8 MIU IFNβ-1b

| | | | | | | | | |
|---|---|---|---|---|---|---|---|---|
| female | Age | 14 | 0 | 51.98 | 11.31 | 31.8 | 52.52 | 66.2 |
| | [years] | | | | | | | |
| | Weight | 11 | 3 | 64.14 | 10.17 | 52.0 | 62.00 | 90.0 |
| | [kg] | | | | | | | |
| | Height | 12 | 2 | 164.92 | 6.69 | 158.0 | 164.50 | 180.0 |
| | [cm] | | | | | | | |
| | BMI | 11 | 3 | 23.88 | 2.63 | 20.8 | 23.81 | 30.8 |
| | [kg/m²] | | | | | | | |
| male | Age | 4 | 0 | 55.06 | 4.91 | 51.7 | 53.13 | 62.2 |
| | [years] | | | | | | | |
| | Weight | 4 | 0 | 77.50 | 7.00 | 69.0 | 78.00 | 85.0 |
| | [kg] | | | | | | | |
| | Height | 4 | 0 | 171.00 | 10.55 | 160.0 | 169.50 | 185.0 |
| | [cm] | | | | | | | |
| | BMI | 4 | 0 | 26.66 | 3.30 | 21.9 | 27.84 | 29.0 |
| | [kg/m²] | | | | | | | |

Overall

| | | | | | | | |
|---|---|---|---|---|---|---|---|
| Age | 32 | 0 | 53.18 | 11.06 | 29.7 | 53.95 | 76.2 |
| [years] | | | | | | | |
| Weight | 24 | 8 | 72.92 | 14.73 | 51.0 | 70.50 | 109.8 |
| [kg] | | | | | | | |
| Height | 26 | 6 | 169.58 | 8.29 | 158.0 | 168.50 | 187.0 |
| [cm] | | | | | | | |
| BMI | 23 | 9 | 25.41 | 4.31 | 18.1 | 24.22 | 39.4 |
| [kg/m²] | | | | | | | |

TABLE 6
Total duration of betaferon treatment - All randomized patients

| | Treatment | N | Nmiss | Mean | SD | Min | Median | Max |
|---|---|---|---|---|---|---|---|---|
| Dura- | 4 MIU | 13 | 1 | 172.5 | 7.3 | 158 | 169.0 | 186 |
| tion | IFNβ-1b | | | | | | | |
| [days] | 8 MIU | 18 | 0 | 165.9 | 30.7 | 51 | 169.5 | 194 |
| | IFNβ-1b | | | | | | | |
| | Overall | 31 | 1 | 168.7 | 23.8 | 51 | 169.0 | 194 |

Analyses of Parvovirus B19 and HHV6 in Endomyocardial Biopsy

Table 7 shows analyses for Parvovirus B19 and HHV6 in endomyocardial biopsy in patients of the 4 MIU group, in whom virus analyses are available prior to study treatment and after the end of treatment. In patients 06 and 10 no analyses for Parvovirus B19 and HHV6 was done after the end of study treatment. In patient 32 the results are still pending.

TABLE 7
Analyses for Parvovirus B19 and HHV6 in patients (4 MIU group) in
whom virus analyses are available prior to study treatment and after the
end of treatment
4 MIU group

| | Parvovirus B19, | | | |
|---|---|---|---|---|
| | qualitative (pos./neg.)/ | | | |
| | quantitative (viral copies | | HHV6, qualitative | |
| | per μg myocardial | | (pos./neg.) | |
| | nucleic acid) | | Prior to | |
| | Prior to study | After study | study | After study |
| Patient | treatment | treatment | treatment | treatment |
| 02 | Pos | 43 | Pos | Neg |
| 04 | 250.000 | 220 | Neg | Neg |
| 08 | Neg | Neg | Pos | Pos |
| 14 | Neg | Neg | Pos | Pos |
| 16 | 242 | 358 | Neg | Pos |
| 19 | 1.100 | Pos | Neg | Pos |
| 20 | 1250 | Pos | Neg | Neg |
| 22 | Pos | Pos | Neg | Neg |
| 26 | 814 | 808 | Neg | Neg |
| 27 | 340 | 679 | Neg | Neg |
| 31 | Pos | Pos | Neg | Neg |

In 2 out of 9 patients (20.0%) of the 4 MIU group who were positive for Parvovirus B19 prior to study treatment there was absence of Parvovirus or reduction of Parvovirus load detectable in the endomyocardial biopsy after the end of study treatment. In 2 out of 10 patients of the 4 MIU group who were positive for Parvovirus B19 prior to study treatment there was an increase Parvovirus load detectable in the endomyocardial biopsy after the end of study treatment.

In 1 out of 3 patients (33.3%) of the 4 MIU group who were positive for HHV6 prior to study treatment there was absence HHV6 detectable in the endomyocardial biopsy after the end of study treatment. One patient with HHV6 prior to study treatment had also Parvovirus B19 in the endomyocardial biopsy prior to study treatment. There were two patients who was negative for HHV6 in the endomyocardial biopsy prior to study treatment, but positive after the end of treatment.

Table 8 shows analyses for Parvovirus B19 and HHV6 in endomyocardial biopsy in patients of the 8 MIU group, in whom virus analyses are available prior to study treatment and after the end of treatment. In patient 13 no analyses for Parvovirus B19 and HHV6 was done after the end of study treatment.

In 7 out of 15 patients (46.6%) of the 8 MIU group who were positive for Parvovirus B19 prior to study treatment there was absence of Parvovirus or reduction of Parvovirus load detectable in the endomyocardial biopsy after the end of study treatment. In 1 out of 13 patients of the 8 MIU group who were positive for Parvovirus B19 prior to study treatment there was an increase of Parvovirus load detectable in the endomyocardial biopsy after the end of study treatment.

In 6 out of 7 patients (85.7%) of the 8 MIU group who were positive for HHV6 prior to study treatment (and in whom HHV6 results in the endomyocardial biopsy are available pior to and after study treatment) there was absence HHV6 detectable in the endomyocardial biopsy after the end of study treatment. In one patient who was positive for HHV6 prior to study treatment there was no biopsy result for HHV6 available. Six out of the 8 patients with HHV6 prior to study treatment had also Parvovirus B19 in the endomyocardial biopsy prior to study treatment.

TABLE 8
Analyses for Parvovirus B19 and HHV6 in patients (8 MIU group) in
whom virus analyses are available prior to study treatment and after
the end of treatment
8 MIU group

| | Parvovirus B19, | | | |
|---|---|---|---|---|
| | qualitative (pos./neg.)/ | | | |
| | quantitative (viral copies | | | |
| | per μg myocardial | | HHV6, qualitative | |
| Patient | nucleic acid) | | (pos./neg.) | |
| 01 | 1.900 | 115 | Neg | Neg |
| 03 | Pos | Pos | Neg | Neg |
| 05 | 674 | 307 | Pos | Neg |
| 07 | 1.200 | 792 | Pos | Pos |
| 09 | 150 | Neg | Pos | Neg |
| 11 | 73 | 280 | Not available | Neg |
| 12 | 704 | Neg | Pos | Neg |
| 15 | 1.100 | 119 | Neg | Neg |
| 17 | Pos | Pos | Neg | Neg |
| 18 | 402 | 300 | Neg | Neg |
| 21 | Pos | Pos | Neg | Neg |
| 23 | Neg | Neg | Pos | Neg |
| 24 | 1.700 | Pos | Neg | Neg |
| 25 | Pos | 125 | Neg | Neg |
| 28 | Pos | 238 | Pos | Neg |
| 29 | Neg | Neg | Pos | Neg |
| 30 | Pos | 95 | Neg | Neg |

Summary

In patients, in whom virus analyses are available prior to study treatment and after the end of treatment, detectable absence of Parvovirus or reduction of Parvovirus load in the endomyocardial biopsy after the end of study treatment was more pronounced in the 8 MIU compared to the 4 MIU group (46.6 vs 20.0%). In patients, in whom virus analyses are available prior to study treatment and after the end of treatment, detectable absence of HHV6 in the endomyocardial biopsy after the end of study treatment was more pronounced in the 8 MIU compared to the 4 MIU group (85.7 vs 33.3%).

Clinical Parameters

Table 9 shows changes in NYHA functional class from baseline to follow-up week 12 and 24 for patients who have a baseline value and at least one NYHA functional class assessment after the end of treatment.

In total, 6 out of 12 patients (50.0%) of the 4 MIU group and 7 out of 17 patients (41.1%) of the 8 MIU group have shown improvement regarding the NYHA functional class at the time of the last assessment of this parameters. This improvement was slightly more pronounced in the 8 MIU group in which 2 patients showed an improvement by 2 classes compared to baseline.

TABLE 9
NYHA class - Shift tables (Baseline - FUP wk 12 and 24) - Treatment
completers

| | | | Treatment | |
|---|---|---|---|---|
| | | NYHA | 4 MIU IFN | 8 MIU IFN |
| | | classification | beta-1b | beta-1b |
| | Baseline | of angina | Number of | Number of |
| Study phase | value | severity | patients | patients |
| Last | I | I | 0 (0.0%) | 0 (0.0%) |
| available | | II | 0 (0.0%) | 0 (0.0%) |
| visit | | III | 0 (0.0%) | 0 (0.0%) |
| | | IV | 0 (0.0%) | 0 (0.0%) |
| | | Total | 0 (0.0%) | 0 (0.0%) |
| | II | I | 3 (50.0%) | 2 (28.6%) |
| | | II | 3 (50.0%) | 4 (57.1%) |
| | | III | 0 (0.0%) | 1 (14.3%) |
| | | IV | 0 (0.0%) | 0 (0.0%) |
| | | Total | 6 (100.0%) | 7 (100.0%) |
| | III | I | 0 (0.0%) | 2 (20.0%) |
| | | II | 3 (50.0%) | 3 (30.0%) |
| | | III | 3 (50.0%) | 5 (50.0%) |
| | | IV | 0 (0.0%) | 0 (0.0%) |
| | | Total | 6 (100.0%) | 10 (100.0%) |
| | IV | I | 0 (0.0%) | 0 (0.0%) |
| | | II | 0 (0.0%) | 0 (0.0%) |
| | | III | 0 (0.0%) | 0 (0.0%) |
| | | IV | 0 (0.0%) | 0 (0.0%) |
| | | Total | 0 (0.0%) | 0 (0.0%) |

One patient of the 8 MIU group showed a deterioration in NYHA functional class after the end of treatment.

Table 10 shows changes in composite clinical end point from baseline to follow-up week 12 and 24 for patients who have a baseline value and at least one assessment of this parameter after the end of treatment.

TABLE 10
Composite clinical endpoint - Frequency table - Treatment completers

| | | | 8 MIU IFN | 4 MIU IFN |
|---|---|---|---|---|
| | | | beta-1b | beta-1b |
| Treatment | Period of | | Number of | Number of |
| Study phase | interest | Assessment | patients | patients |
| Last | since baseline | improved | 8 (66.7%) | 11 (64.7%) |
| available | | unchanged | 4 (33.3%) | 5 (29.4%) |
| visit | | worsened | 0 (0.0%) | 1 (5.9%) |
| | | Total | 12 (100.0%) | 17 (100.0%) |

In total, 8 out of 12 patients (66.7%) of the 4 MIU group and 11 out of 17 patients (64.7%) of the 8 MIU group have shown improvement regarding the composite clinical end point at the time of the last assessment of this parameter. One patient of the 8 MIU showed a deterioration after the end of study treatment.

Table 11 shows changes in patient global assessment from baseline to follow-up week 12 and 24 for patients who have a baseline value and at least one assessment of this parameter after the end of treatment.

TABLE 11
Patient global assessment - Frequency table - Treatment completers
Treatment

| | | | 8 MIU IFN | 4 MIU IFN |
|---|---|---|---|---|
| | | | beta-1b | beta-1b |
| Study | Period of | Overall | Number of | Number of |
| phase | interest | status | patients | patients |
| Last | since | very much improved | 2 (20.0%) | 3 (17.6%) |
| available | baseline | much improved | 4 (40.0%) | 7 (41.2%) |
| visit | | minimally improved | 2 (20.0%) | 4 (23.5%) |
| | | no change | 2 (20.0%) | 2 (11.8%) |
| | | minimally worse | 0 (0.0%) | 1 (5.9%) |
| | | Total | 10 (100.0%) | 17 (100.0%) |

In total, 8 out of 10 patients (80.0%) of the 4 MIU group and 14 out of 17 patients (82.3%) of the 8 MIU group have shown improvement regarding the patient global assessment at the time of the last assessment of this parameters. One patient of the 8 MIU showed a slight deterioration after the end of study treatment.

Three out of 5 patients (60.0%) of the 4 MIU group and 3 out of 5 patients (60.0%) of the 8 MIU group showed an improvement regarding quality of life (assessed by the Minnesota Living with Heart Failure Questionnaire) from baseline to follow-up week 12 and 24 for patients who have a baseline value and at least one assessment of this parameter after the end of treatment.

Summary

Improvement regarding NYHA functional class, composite clinical endpoint, patient global assessment and quality of life is comparable in both dose groups. The improvement in clinial outcome can be the result of improvement cardiac function induced by treatment with IFNβ-1b.

Left Ventricular Ejection Fraction

Table 12 shows the changes in LVEF at rest and on exertion from baseline to treatment week 24, follow-up week 12 after the end of treatment (Table 4.2.1). The mean LVEF at rest and on exertion changed only slightly in both dose groups.

Since there were many missing values for LVEF determined by RNV, a review of medical records evaluating all determinations of LVEF (regardless by which method the LVEF was determined) during a study period of the respective patients was done. For this evaluation only values were used which were close to baseline and/or the planned visit for the control LVEF determination (12 weeks after the end of treatment). This evaluation revealed a median increase of LVEF at rest by 3.5% and a mean increase by 5.9% in the 4 MIU group (n=12 compared to n=8 only for LVEF determined by RNV). In the 8 MIU group the median LVEF increase was 4.5% and the mean increase was 7.3% (n=16 compared to n=9 only for LVEF determined by RNV). The baseline LVEF values used for this extended evaluation, were determined in average 2.5 (4 MIU group) and 2.1 months (8 MIU group) prior to randomization. It should also be considered, that this evaluation contains LVEF values obtained by different methods for RNV determination.

Summary

The increases in LVEF can be interpreted as an improvement of left ventricular systolic function after re-remodelling induced by treatment with IFNβ-1b.

TABLE 12
LVEF at rest and on exertion (40 W, 70 W) - Treatment completers

| | Procedure | | | | | | | | | | | | | | |
|---|---|---|---|---|---|---|---|---|---|---|---|---|---|---|---|
| Study | sequence | Measured values | | | | | | | | | Changes from baseline | | | | |
| phase | key item | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | unstressed | 8 | 6 | 31.8 | 13.3 | 21 | 27.0 | 62 | 0 | 14 | | | | | |
| | first workload | 7 | 7 | 25.6 | 8.3 | 15 | 22.0 | 37 | 0 | 14 | | | | | |
| | (40 W) | | | | | | | | | | | | | | |
| | second workload | 5 | 9 | 33.0 | 17.5 | 15 | 37.0 | 56 | 0 | 14 | | | | | |
| | (70 W) | | | | | | | | | | | | | | |
| Follow-up | unstressed | 8 | 6 | 35.1 | 14.0 | 23 | 31.5 | 65 | 8 | 6 | 3.4 | 7.9 | −5 | 2.5 | 21 |
| Week 12 | first workload | 7 | 7 | 27.9 | 8.2 | 14 | 30.0 | 37 | 7 | 7 | 2.3 | 7.7 | −8 | 0.0 | 14 |
| | (40 W) | | | | | | | | | | | | | | |
| | second workload | 5 | 9 | 35.4 | 16.3 | 23 | 30.0 | 64 | 5 | 9 | 2.4 | 11.5 | −11 | 7.0 | 16 |
| | (70 W) | | | | | | | | | | | | | | |

8 MIU IFNβ-1b

| | | | | | | | | | | | | | | | |
|---|---|---|---|---|---|---|---|---|---|---|---|---|---|---|---|
| Baseline | unstressed | 9 | 9 | 35.2 | 9.7 | 23 | 36.0 | 46 | 0 | 18 | | | | | |
| | first workload | 9 | 9 | 32.8 | 8.3 | 18 | 31.0 | 44 | 0 | 18 | | | | | |
| | (40 W) | | | | | | | | | | | | | | |
| | second workload | 8 | 10 | 31.9 | 9.7 | 19 | 31.0 | 49 | 0 | 18 | | | | | |
| | (70 W) | | | | | | | | | | | | | | |
| Follow-up | unstressed | 9 | 9 | 33.2 | 7.0 | 23 | 34.0 | 45 | 9 | 9 | −2.0 | 5.3 | −10 | −2.0 | 5 |
| Week 12 | first workload | 9 | 9 | 34.4 | 10.4 | 21 | 35.0 | 51 | 9 | 9 | 1.7 | 7.9 | −7 | −3.0 | 14 |
| | (40 W) | | | | | | | | | | | | | | |
| | second workload | 8 | 10 | 33.1 | 7.7 | 23 | 31.5 | 43 | 8 | 10 | 1.3 | 5.8 | −6 | 0.5 | 11 |
| | (70 W) | | | | | | | | | | | | | | |

Echocardiographic Parameters

The following evaluation of echocardiographic parameters differentiates between patients with a left ventricular enddiastolic diameter (LVEDD)≦60 mm and >60 mm at baseline, and a left ventricular endsystolic diameter (LVESD)≦50 mm and >50 mm at baseline. These cut-off values were used to evaluate effect of study treatment on changes of the parameters in patients with a more dilated left ventricle and patients with a rather non-dilated left ventricle at baseline.

Table 13 shows the changes in LVEDD from baseline to treatment week 24, and follow-up weeks 12 and 24 after the end of treatment in patients with a LVEDD≦60 mm at baseline. In the 4 MIU group there was a mean change of LVEDD from baseline to the last available visit by 0.0 mm to −5.3 mm compared. In the 8 MIU group there was a mean change of LVEDD by −0.5 to −2.0 mm compared to baseline.

Table 14 shows the changes in LVEDD from baseline to treatment week 24, and follow-up weeks 12 and 24 after the end of treatment in patients with a LVEDD>60 mm at baseline. In the 4 MIU group there was a mean change of LVEDD by −1.3 to −3.0 mm compared to baseline. In the 8 MIU group there was a mean change of LVEDD by −0.8 to −2.1 mm compared to baseline.

Table 15 shows the changes in LVESD from baseline to treatment week 24, and follow-up weeks 12 and 24 after the end of treatment in patients with a LVEDD≦60 mm at baseline. In the 4 MIU group there was a mean change of LVESD by −2.0 to −5.5 mm compared to baseline. In the 8 MIU group there was a mean change of LVESD by −1.6 to −2.0 mm compared to baseline.

Table 16 shows the changes in LVESD from baseline to treatment week 24, and follow-up weeks 12 and 24 after the end of treatment in patients with a LVEDD>60 mm at baseline. In the 4 MIU group there was a mean change of LVESD by 0.2 to −2.0 mm compared to baseline. In the 8 MIU group there was a mean change of LVESD by 0.0 to −2.0 mm compared to baseline.

TABLE 13
Echocardiography: LVEDD [mm] - Treatment completers - LVEDD <= 60 mm at baseline

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 4 | 0 | 53.5 | 4.1 | 50 | 53.0 | 58 | 0 | 4 | | | | | |
| Treatment | Week 24 | 4 | 0 | 48.3 | 6.1 | 43 | 46.5 | 57 | 4 | 0 | −5.3 | 3.5 | −9 | −5.5 | −1 |
| Baseline | | 1 | 3 | 50.0 | 50 | 50.0 | 50 | 0 | 4 | | | | | | |
| Follow up | Week 12 | 1 | 3 | 50.0 | 50 | 50.0 | 50 | 1 | 3 | 0.0 | 0 | 0.0 | 0 | | |
| Baseline | | 1 | 3 | 50.0 | 50 | 50.0 | 50 | 0 | 4 | | | | | | |
| Follow up | Week 24 | 1 | 3 | 45.0 | 45 | 45.0 | 45 | 1 | 3 | −5.0 | −5 | −5.0 | −5 | | |
| Baseline | | 4 | 0 | 53.5 | 4.1 | 50 | 53.0 | 58 | 0 | 4 | | | | | |
| Last available visit | | 4 | 0 | 49.8 | 5.3 | 45 | 48.5 | 57 | 4 | 0 | −3.8 | 4.1 | −9 | −3.0 | 0 |

8 MIU IFNβ-1b

| | | | | | | | | | | | | | | | |
|---|---|---|---|---|---|---|---|---|---|---|---|---|---|---|---|
| Baseline | | 10 | 0 | 50.8 | 6.4 | 40 | 51.0 | 59 | 0 | 10 | | | | | |
| Treatment | Week 24 | 10 | 0 | 50.3 | 6.0 | 38 | 51.5 | 59 | 10 | 0 | −0.5 | 2.5 | −4 | −0.5 | 3 |
| Baseline | | 7 | 3 | 48.7 | 6.5 | 40 | 49.0 | 58 | 0 | 10 | | | | | |
| Follow up | Week 12 | 7 | 3 | 47.1 | 5.4 | 37 | 48.0 | 54 | 7 | 3 | −1.6 | 3.3 | −8 | −1.0 | 2 |
| Baseline | | 5 | 5 | 51.6 | 4.6 | 45 | 53.0 | 56 | 0 | 10 | | | | | |
| Follow up | Week 24 | 5 | 5 | 49.6 | 4.3 | 44 | 52.0 | 54 | 5 | 5 | −2.0 | 2.0 | −4 | −3.0 | 1 |
| Baseline | | 10 | 0 | 50.8 | 6.4 | 40 | 51.0 | 59 | 0 | 10 | | | | | |
| Last available visit | | 10 | 0 | 48.8 | 6.1 | 37 | 49.0 | 59 | 10 | 0 | −2.0 | 2.9 | −8 | −2.0 | 2 |

TABLE 14
Echocardiography: LVEDD [mm] - Treatment completers - LVEDD > 60 mm at baseline

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 8 | 2 | 68.6 | 4.0 | 63 | 68.5 | 74 | 0 | 10 | | | | | |
| Treatment | Week 24 | 8 | 2 | 67.1 | 4.2 | 60 | 68.0 | 72 | 8 | 2 | −1.5 | 4.2 | −6 | −3.0 | 5 |
| Baseline | | 9 | 1 | 70.7 | 5.8 | 63 | 69.0 | 79 | 0 | 10 | | | | | |
| Follow up | Week 12 | 9 | 1 | 67.7 | 5.0 | 59 | 69.0 | 76 | 9 | 1 | −3.0 | 4.6 | −9 | −3.0 | 6 |
| Baseline | | 6 | 4 | 71.2 | 5.6 | 63 | 71.5 | 79 | 0 | 10 | | | | | |
| Follow up | Week 24 | 6 | 4 | 69.8 | 2.8 | 67 | 69.5 | 75 | 6 | 4 | −1.3 | 4.6 | −6 | −2.5 | 7 |
| Baseline | | 10 | 0 | 70.6 | 5.5 | 63 | 69.5 | 79 | 0 | 10 | | | | | |
| Last available visit | | 10 | 0 | 68.8 | 3.6 | 63 | 69.5 | 75 | 10 | 0 | −1.8 | 4.2 | −8 | −2.0 | 7 |

8 MIU IFNβ-1b

| | | | | | | | | | | | | | | | |
|---|---|---|---|---|---|---|---|---|---|---|---|---|---|---|---|
| Baseline | | 7 | 1 | 64.7 | 3.5 | 62 | 63.0 | 71 | 0 | 8 | | | | | |
| Treatment | Week 24 | 7 | 1 | 62.6 | 4.2 | 57 | 63.0 | 70 | 7 | 1 | −2.1 | 2.6 | −6 | −3.0 | 1 |
| Baseline | | 6 | 2 | 65.2 | 3.5 | 62 | 63.5 | 71 | 0 | 8 | | | | | |
| Follow up | Week 12 | 6 | 2 | 63.5 | 5.2 | 58 | 63.0 | 70 | 6 | 2 | −1.7 | 3.1 | −6 | −0.5 | 1 |
| Baseline | | 4 | 4 | 65.0 | 4.1 | 62 | 63.5 | 71 | 0 | 8 | | | | | |
| Follow up | Week 24 | 4 | 4 | 64.3 | 5.2 | 61 | 62.0 | 72 | 4 | 4 | −0.8 | 1.3 | −2 | −1.0 | 1 |
| Baseline | | 7 | 1 | 64.7 | 3.5 | 62 | 63.0 | 71 | 0 | 8 | | | | | |
| Last available visit | | 7 | 1 | 63.7 | 4.9 | 58 | 62.0 | 72 | 7 | 1 | −1.0 | 2.1 | −5 | −1.0 | 1 |

TABLE 15
Echocardiography: LVESD [mm] - Treatment completers - LVEDD <= 60 mm at baseline

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 4 | 0 | 37.3 | 8.1 | 28 | 38.0 | 45 | 0 | 4 | | | | | |
| Treatment | Week 24 | 4 | 0 | 31.8 | 8.7 | 22 | 32.5 | 40 | 4 | 0 | −5.5 | 1.7 | −7 | −6.0 | −3 |
| Baseline | | 1 | 3 | 33.0 | | | 33 | 33.0 | 33 | 0 | 4 | | | | |
| Follow up | Week 12 | 1 | 3 | 28.0 | | | 28 | 28.0 | 28 | 1 | 3 | −5.0 | −5 | −5.0 | −5 |
| Baseline | | 1 | 3 | 28.0 | | | 28 | 28.0 | 28 | 0 | 4 | | | | |
| Follow up | Week 24 | 1 | 3 | 26.0 | | | 26 | 26.0 | 26 | 1 | 3 | −2.0 | −2 | −2.0 | −2 |
| Baseline | | 4 | 0 | 37.3 | 8.1 | 28 | 38.0 | 45 | 0 | 4 | | | | | |
| Last available visit | | 4 | 0 | 33.0 | 7.0 | 26 | 33.0 | 40 | 4 | 0 | −4.3 | 2.2 | −7 | −4.0 | −2 |

8 MIU IFNβ-1b

| | | | | | | | | | | | | | | | |
|---|---|---|---|---|---|---|---|---|---|---|---|---|---|---|---|
| Baseline | | 9 | 0 | 35.8 | 9.7 | 21 | 37.0 | 47 | 0 | 9 | | | | | |
| Treatment | Week 24 | 9 | 0 | 34.2 | 8.4 | 19 | 37.0 | 47 | 9 | 0 | −1.6 | 3.2 | −7 | −1.0 | 3 |
| Baseline | | 6 | 3 | 33.2 | 10.7 | 21 | 30.0 | 47 | 0 | 9 | | | | | |
| Follow up | Week 12 | 6 | 3 | 31.2 | 8.6 | 18 | 32.0 | 40 | 6 | 3 | −2.0 | 4.2 | −8 | −2.0 | 3 |
| Baseline | | 5 | 4 | 36.2 | 6.7 | 27 | 37.0 | 45 | 0 | 9 | | | | | |
| Follow up | Week 24 | 5 | 4 | 34.2 | 5.4 | 25 | 36.0 | 39 | 5 | 4 | −2.0 | 4.3 | −9 | −2.0 | 2 |
| Baseline | | 9 | 0 | 35.8 | 9.7 | 21 | 37.0 | 47 | 0 | 9 | | | | | |
| Last available visit | | 9 | 0 | 33.7 | 8.7 | 18 | 36.0 | 47 | 9 | 0 | −2.1 | 4.0 | −9 | −2.0 | 2 |

TABLE 16
Echocardiography: LVESD [mm] − Treatment completers − LVEDD > 60 mm at baseline

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 5 | 4 | 52.4 | 2.9 | 48 | 53.0 | 56 | 0 | 9 | | | | | |
| Treatment | Week 24 | 5 | 4 | 52.6 | 8.0 | 41 | 55.0 | 60 | 5 | 4 | 0.2 | 6.1 | −7 | −1.0 | 7 |
| Baseline | | 7 | 2 | 54.9 | 6.9 | 48 | 53.0 | 68 | 0 | 9 | | | | | |
| Follow up | Week 12 | 7 | 2 | 52.9 | 10.1 | 38 | 55.0 | 67 | 7 | 2 | −2.0 | 6.1 | −14 | −1.0 | 5 |
| Baseline | | 6 | 3 | 57.5 | 6.5 | 52 | 54.5 | 68 | 0 | 9 | | | | | |
| Follow up | Week 24 | 6 | 3 | 56.0 | 6.4 | 49 | 54.5 | 68 | 6 | 3 | −1.5 | 4.2 | −9 | −0.5 | 3 |
| Baseline | | 9 | 0 | 55.9 | 6.5 | 48 | 53.0 | 68 | 0 | 9 | | | | | |
| Last available visit | | 9 | 0 | 54.2 | 7.1 | 43 | 54.0 | 68 | 9 | 0 | −1.7 | 3.6 | −9 | −1.0 | 3 |

8 MIU IFNβ-1b

| | | | | | | | | | | | | | | | |
|---|---|---|---|---|---|---|---|---|---|---|---|---|---|---|---|
| Baseline | | 6 | 2 | 50.2 | 4.4 | 43 | 50.5 | 55 | 0 | 8 | | | | | |
| Treatment | Week 24 | 6 | 2 | 48.2 | 5.8 | 43 | 47.0 | 59 | 6 | 2 | −2.0 | 7.1 | −12 | −1.5 | 6 |
| Baseline | | 5 | 3 | 51.6 | 3.0 | 48 | 52.0 | 55 | 0 | 8 | | | | | |
| Follow up | Week 12 | 5 | 3 | 49.8 | 4.4 | 45 | 48.0 | 55 | 5 | 3 | −1.8 | 5.8 | −10 | 0.0 | 5 |
| Baseline | | 4 | 4 | 51.0 | 5.5 | 43 | 53.0 | 55 | 0 | 8 | | | | | |
| Follow up | Week 24 | 4 | 4 | 49.8 | 5.9 | 42 | 50.5 | 56 | 4 | 4 | −1.3 | 2.4 | −3 | −2.0 | 2 |
| Baseline | | 6 | 2 | 50.2 | 4.4 | 43 | 50.5 | 55 | 0 | 8 | | | | | |
| Last available visit | | 6 | 2 | 50.2 | 5.0 | 42 | 50.5 | 56 | 6 | 2 | 0.0 | 3.1 | −3 | −0.5 | 5 |

Table 17 shows the changes in LVEDD from baseline to treatment week 24, and follow-up weeks 12 and 24 after the end of treatment in patients with a LVESD≦50 mm at baseline (Table 7.3.2.1.1). In the 4 MIU group there was a mean change of LVEDD by −1.3 mm to −5.0 mm compared to baseline. In the 8 MIU group there was a mean change of LVEDD by −1.2 to −1.8 mm compared to baseline.

Table 18 shows the changes in LVEDD from baseline to treatment week 24, and follow-up weeks 12 and 24 after the end of treatment in patients with a LVESD>50 mm at baseline. In the 4 MIU group there was a mean change of LVEDD by −1.3 to −3.3 mm compared to baseline. In the 8 MIU group there was a mean change of LVEDD by −0.7 to −2.7 mm compared to baseline.

Table 19 shows the changes in LVESD from baseline to treatment week 24, and follow-up weeks 12 and 24 after the end of treatment in patients with a LVESD≦50 mm at baseline. In the 4 MIU group there was a mean change of LVESD by −2.0 to −5.8 mm compared to baseline. In the 8 MIU group there was a mean change of LVESD by −0.9 to −1.8 mm compared to baseline.

Table 20 shows the changes in LVESD from baseline to treatment week 24, and follow-up weeks 12 and 24 after the end of treatment in patients with a LVESD>50 mm at baseline. In the 4 MIU group there was a mean change of LVESD by 2.0 to −1.6 mm compared to baseline. In the 8 MIU group there was a mean change of LVESD by −1.3 to −5.0 mm compared to baseline.

TABLE 17
Echocardiography: LVEDD [mm] − Treatment completers − LVESD <= 50 mm at baseline

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 6 | 0 | 57.5 | 7.0 | 50 | 57.0 | 66 | 0 | 6 | | | | | |
| Treatment | Week 24 | 6 | 0 | 52.8 | 8.6 | 43 | 52.0 | 64 | 6 | 0 | −4.7 | 3.0 | −9 | −4.5 | −1 |
| Baseline | | 3 | 3 | 60.3 | 9.0 | 50 | 65.0 | 66 | 0 | 6 | | | | | |
| Follow up | Week 12 | 3 | 3 | 59.0 | 7.8 | 50 | 63.0 | 64 | 3 | 3 | −1.3 | 1.2 | −2 | −2.0 | 0 |
| Baseline | | 1 | 5 | 50.0 | | 50 | 50.0 | 50 | 0 | 6 | | | | | |
| Follow up | Week 24 | 1 | 5 | 45.0 | | 45 | 45.0 | 45 | 1 | 5 | −5.0 | | −5 | −5.0 | −5 |
| Baseline | | 6 | 0 | 57.5 | 7.0 | 50 | 57.0 | 66 | 0 | 6 | | | | | |
| Last available visit | | 6 | 0 | 54.3 | 8.2 | 45 | 53.5 | 64 | 6 | 0 | −3.2 | 3.3 | −9 | −2.0 | 0 |

8 MIU IFNβ-1b

| | | | | | | | | | | | | | | | |
|---|---|---|---|---|---|---|---|---|---|---|---|---|---|---|---|
| Baseline | | 13 | 0 | 54.9 | 8.4 | 40 | 56.0 | 68 | 0 | 13 | | | | | |
| Treatment | Week 24 | 13 | 0 | 53.8 | 7.9 | 38 | 54.0 | 65 | 13 | 0 | −1.2 | 2.6 | −6 | −1.0 | 2 |
| Baseline | | 9 | 4 | 53.9 | 9.8 | 40 | 56.0 | 68 | 0 | 13 | | | | | |
| Follow up | Week 12 | 9 | 4 | 52.3 | 9.7 | 37 | 50.0 | 69 | 9 | 4 | −1.6 | 3.3 | −8 | 0.0 | 2 |
| Baseline | | 6 | 7 | 53.3 | 5.9 | 45 | 54.0 | 62 | 0 | 13 | | | | | |
| Follow up | Week 24 | 6 | 7 | 51.5 | 6.1 | 44 | 52.0 | 61 | 6 | 7 | −1.8 | 1.8 | −4 | −2.0 | 1 |
| Baseline | | 13 | 0 | 54.9 | 8.4 | 40 | 56.0 | 68 | 0 | 13 | | | | | |
| Last available visit | | 13 | 0 | 53.1 | 8.7 | 37 | 52.0 | 69 | 13 | 0 | −1.8 | 2.8 | −8 | −1.0 | 2 |

TABLE 18
Echocardiography: LVEDD [mm] − Treatment completers − LVESD > 50 mm at baseline

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 5 | 2 | 69.6 | 4.6 | 63 | 69.0 | 74 | 0 | 7 | | | | | |
| Treatment | Week 24 | 5 | 2 | 68.2 | 2.9 | 64 | 68.0 | 72 | 5 | 2 | −1.4 | 5.0 | −6 | −4.0 | 5 |
| Baseline | | 7 | 0 | 72.1 | 5.8 | 63 | 74.0 | 79 | 0 | 7 | | | | | |
| Follow up | Week 12 | 7 | 0 | 68.9 | 5.0 | 59 | 70.0 | 76 | 7 | 0 | −3.3 | 5.3 | −9 | −4.0 | 6 |
| Baseline | | 6 | 1 | 71.2 | 5.6 | 63 | 71.5 | 79 | 0 | 7 | | | | | |
| Follow up | Week 24 | 6 | 1 | 69.8 | 2.8 | 67 | 69.5 | 75 | 6 | 1 | −1.3 | 4.6 | −6 | −2.5 | 7 |
| Baseline | | 7 | 0 | 72.1 | 5.8 | 63 | 74.0 | 79 | 0 | 7 | | | | | |
| Last available visit | | 7 | 0 | 69.9 | 2.5 | 67 | 70.0 | 75 | 7 | 0 | −2.3 | 4.9 | −8 | −4.0 | 7 |

8 MIU IFNβ-1b

| | | | | | | | | | | | | | | | |
|---|---|---|---|---|---|---|---|---|---|---|---|---|---|---|---|
| Baseline | | 3 | 1 | 66.0 | 4.4 | 63 | 64.0 | 71 | 0 | 4 | | | | | |
| Treatment | Week 24 | 3 | 1 | 63.3 | 5.8 | 60 | 60.0 | 70 | 3 | 1 | −2.7 | 1.5 | −4 | −3.0 | −1 |
| Baseline | | 3 | 1 | 66.0 | 4.4 | 63 | 64.0 | 71 | 0 | 4 | | | | | |
| Follow up | Week 12 | 3 | 1 | 64.0 | 6.0 | 58 | 64.0 | 70 | 3 | 1 | −2.0 | 3.6 | −6 | −1.0 | 1 |
| Baseline | | 3 | 1 | 66.0 | 4.4 | 63 | 64.0 | 71 | 0 | 4 | | | | | |
| Follow up | Week 24 | 3 | 1 | 65.3 | 5.8 | 62 | 62.0 | 72 | 3 | 1 | −0.7 | 1.5 | −2 | −1.0 | 1 |
| Baseline | | 3 | 1 | 66.0 | 4.4 | 63 | 64.0 | 71 | 0 | 4 | | | | | |
| Last available visit | | 3 | 1 | 65.3 | 5.8 | 62 | 62.0 | 72 | 3 | 1 | −0.7 | 1.5 | −2 | −1.0 | 1 |

TABLE 19
Echocardiography: LVESD [mm] − Treatment completers − LVESD <= 50 mm at baseline

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 5 | 1 | 39.4 | 8.5 | 28 | 43.0 | 48 | 0 | 6 | | | | | |
| Treatment | Week 24 | 5 | 1 | 33.6 | 8.6 | 22 | 38.0 | 41 | 5 | 1 | −5.8 | 1.6 | −7 | −6.0 | −3 |
| Baseline | | 3 | 3 | 43.7 | 9.3 | 33 | 48.0 | 50 | 0 | 6 | | | | | |
| Follow up | Week 12 | 3 | 3 | 40.0 | 10.8 | 28 | 43.0 | 49 | 3 | 3 | −3.7 | 2.3 | −5 | −5.0 | −1 |
| Baseline | | 1 | 5 | 28.0 | | | 28 | 28.0 | 28 | 0 | 6 | | | | |
| Follow up | Week 24 | 1 | 5 | 26.0 | | | 26 | 26.0 | 26 | 1 | 5 | −2.0 | −2 | −2.0 | −2 |
| Baseline | | 6 | 0 | 41.2 | 8.8 | 28 | 44.0 | 50 | 0 | 6 | | | | | |
| Last available visit | | 6 | 0 | 37.3 | 8.8 | 26 | 39.0 | 49 | 6 | 0 | −3.8 | 2.2 | −7 | −4.0 | −1 |

8 MIU IFNβ-1b

| | | | | | | | | | | | | | | | |
|---|---|---|---|---|---|---|---|---|---|---|---|---|---|---|---|
| Baseline | | 12 | 1 | 38.5 | 9.7 | 21 | 41.0 | 49 | 0 | 13 | | | | | |
| Treatment | Week 24 | 12 | 1 | 37.6 | 9.4 | 19 | 38.0 | 49 | 12 | 1 | −0.9 | 3.5 | −7 | −1.0 | 6 |
| Baseline | | 8 | 5 | 37.0 | 11.5 | 21 | 39.0 | 49 | 0 | 13 | | | | | |
| Follow up | Week 12 | 8 | 5 | 36.1 | 11.8 | 18 | 37.5 | 54 | 8 | 5 | −0.9 | 4.3 | −8 | −0.5 | 5 |
| Baseline | | 6 | 7 | 37.3 | 6.6 | 27 | 38.0 | 45 | 0 | 13 | | | | | |
| Follow up | Week 24 | 6 | 7 | 35.5 | 5.8 | 25 | 36.5 | 42 | 6 | 7 | −1.8 | 3.9 | −9 | −1.5 | 2 |
| Baseline | | 12 | 1 | 38.5 | 9.7 | 21 | 41.0 | 49 | 0 | 13 | | | | | |
| Last available visit | | 12 | 1 | 37.3 | 10.2 | 18 | 38.0 | 54 | 12 | 1 | −1.3 | 4.0 | −9 | −0.5 | 5 |

TABLE 20
Echocardiography: LVESD diameter [mm] − Treatment completers − LVESD > 50 mm at baseline

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 4 | 3 | 53.5 | 1.7 | 52 | 53.0 | 56 | 0 | 7 | | | | | |
| Treatment | Week 24 | 4 | 3 | 55.5 | 5.4 | 48 | 57.0 | 60 | 4 | 3 | 2.0 | 5.4 | −4 | 2.5 | 7 |
| Baseline | | 5 | 2 | 57.2 | 6.8 | 52 | 53.0 | 68 | 0 | 7 | | | | | |
| Follow up | Week 12 | 5 | 2 | 55.6 | 10.8 | 38 | 58.0 | 67 | 5 | 2 | −1.6 | 7.3 | −14 | 0.0 | 5 |
| Baseline | | 6 | 1 | 57.5 | 6.5 | 52 | 54.5 | 68 | 0 | 7 | | | | | |
| Follow up | Week 24 | 6 | 1 | 56.0 | 6.4 | 49 | 54.5 | 68 | 6 | 1 | −1.5 | 4.2 | −9 | −0.5 | 3 |
| Baseline | | 7 | 0 | 57.9 | 6.0 | 52 | 56.0 | 68 | 0 | 7 | | | | | |
| Last available visit | | 7 | 0 | 56.6 | 6.0 | 49 | 55.0 | 68 | 7 | 0 | −1.3 | 3.9 | −9 | 0.0 | 3 |

8 MIU IFNβ-1b

| | | | | | | | | | | | | | | | |
|---|---|---|---|---|---|---|---|---|---|---|---|---|---|---|---|
| Baseline | | 3 | 1 | 53.7 | 1.5 | 52 | 54.0 | 55 | 0 | 4 | | | | | |
| Treatment | Week 24 | 3 | 1 | 48.7 | 9.0 | 43 | 44.0 | 59 | 3 | 1 | −5.0 | 8.9 | −12 | −8.0 | 5 |
| Baseline | | 3 | 1 | 53.7 | 1.5 | 52 | 54.0 | 55 | 0 | 4 | | | | | |
| Follow up | Week 12 | 3 | 1 | 49.0 | 5.3 | 45 | 47.0 | 55 | 3 | 1 | −4.7 | 5.5 | −10 | −5.0 | 1 |
| Baseline | | 3 | 1 | 53.7 | 1.5 | 52 | 54.0 | 55 | 0 | 4 | | | | | |
| Follow up | Week 24 | 3 | 1 | 52.3 | 3.5 | 49 | 52.0 | 56 | 3 | 1 | −1.3 | 2.9 | −3 | −3.0 | 2 |
| Baseline | | 3 | 1 | 53.7 | 1.5 | 52 | 54.0 | 55 | 0 | 4 | | | | | |
| Last available visit | | 3 | 1 | 52.3 | 3.5 | 49 | 52.0 | 56 | 3 | 1 | −1.3 | 2.9 | −3 | −3.0 | 2 |

The following evaluation of echocardiographic parameters differentiates between patients with a LVPW≦9 mm and >9 mm at baseline. This cut-off value were used to evaluate effects of study treatment on changes of the parameters in patients with a more reduced LVPW and patients with a rather non-reduced LVPW at baseline.

Table 21 shows the changes in LVPW from baseline to treatment week 24, and follow-up weeks 12 and 24 after the end of treatment in patients with a LVPW≦9 mm at baseline. In the 4 MIU group there was a mean change of LVPW by 1.5 to 2.0 mm compared to baseline. In the 8 MIU group there was a mean change of LVPW by 0.4 to 1.0 mm compared to baseline.

Table 22 shows the changes in LVPW from baseline to treatment week 24, and follow-up weeks 12 and 24 after the end of treatment in patients with a LVPW>9 mm at baseline. In the 4 MIU group there was a mean change of LVPW by 0.0 to −1.0 mm compared to baseline. In the 8 MIU group there was a mean change of LVPW by −0.1 to −0.4 mm compared to baseline. There was a slight increase of the mean of the E/A ratio from baseline to week 24 after the end of treatment from 1.007 to 1.102 in the 8 MIU group compared to a change from 1.171 to 1.165 in the 4 MIU group.

Summary

Overall, there is a reduction in LVEDD and LVESD after the end of treatment compared to baseline. There was a mean change of LVEDD in the overall 4 MIU group by −1.9 to −2.8 mm compared to −1.2 to −1.6 mm in the overall 8 MIU group. There was a mean change of LVESD in the overall 4 MIU group by −1.6 to −2.4 mm compared to −1.7 to −1.9 mm in the overall 8 MIU group.

The decreases in the left ventricular diameters can be interpreted as re-remodelling of the left ventricle and normalization of the left ventricle size induced by treatment with IFNβ-1b. An increase of the E/A ratio can be interpreted as an improvement in diastolic function. Since there was a slight increase of the mean of the E/A ratio in the 8 MIU group, there are hints that in the 8 MIU group the diastolic function was improved whereas the mean of the E/A ratio in the 4 MIU group remained almost unchanged.

In parallel, the LVPW tends to increase after the end of treatment with IFNβ-1b in patients with a LVPW≦9 mm at baseline. This could be the result of a re-remodelling of the left ventricle induced by treatment with IFNβ-1b. There was a mean change of LVPW in the overall 4 MIU group by 0.0 to 0.9 mm compared to 0.1 to 0.2 mm in the overall 8 MIU group.

TABLE 21
Echocardiography: Left ventricular posterior wall [mm] − Treatment completers − LVPW <= 9 mm at baseline

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 3 | 2 | 8.0 | 0.0 | 8 | 8.0 | 8 | 0 | 5 | | | | | |
| Treatment | Week 24 | 3 | 2 | 10.0 | 1.0 | 9 | 10.0 | 11 | 3 | 2 | 2.0 | 1.0 | 1 | 2.0 | 3 |
| Baseline | | 4 | 1 | 8.3 | 0.5 | 8 | 8.0 | 9 | 0 | 5 | | | | | |
| Follow up | Week 12 | 4 | 1 | 9.8 | 1.7 | 8 | 9.5 | 12 | 4 | 1 | 1.5 | 1.3 | 0 | 1.5 | 3 |
| Baseline | | 3 | 2 | 8.0 | 0.0 | 8 | 8.0 | 8 | 0 | 5 | | | | | |
| Follow up | Week 24 | 3 | 2 | 10.0 | 1.0 | 9 | 10.0 | 11 | 3 | 2 | 2.0 | 1.0 | 1 | 2.0 | 3 |
| Baseline | | 5 | 0 | 8.2 | 0.4 | 8 | 8.0 | 9 | 0 | 5 | | | | | |
| Last available visit | | 5 | 0 | 10.2 | 1.3 | 9 | 10.0 | 12 | 5 | 0 | 2.0 | 1.0 | 1 | 2.0 | 3 |

8 MIU IFNβ-1b

| | | | | | | | | | | | | | | | |
|---|---|---|---|---|---|---|---|---|---|---|---|---|---|---|---|
| Baseline | | 6 | 0 | 8.2 | 0.8 | 7 | 8.0 | 9 | 0 | 6 | | | | | |
| Treatment | Week 24 | 6 | 0 | 9.0 | 0.6 | 8 | 9.0 | 10 | 6 | 0 | 0.8 | 0.8 | 0 | 1.0 | 2 |
| Baseline | | 5 | 1 | 8.0 | 0.7 | 7 | 8.0 | 9 | 0 | 6 | | | | | |
| Follow up | Week 12 | 5 | 1 | 8.4 | 0.5 | 8 | 8.0 | 9 | 5 | 1 | 0.4 | 0.9 | 0 | 0.0 | 2 |
| Baseline | | 4 | 2 | 8.3 | 1.0 | 7 | 8.5 | 9 | 0 | 6 | | | | | |
| Follow up | Week 24 | 4 | 2 | 9.3 | 1.5 | 8 | 9.0 | 11 | 4 | 2 | 1.0 | 1.6 | −1 | 1.0 | 3 |
| Baseline | | 6 | 0 | 8.2 | 0.8 | 7 | 8.0 | 9 | 0 | 6 | | | | | |
| Last available visit | | 6 | 0 | 8.8 | 1.3 | 8 | 8.0 | 11 | 6 | 0 | 0.7 | 1.4 | −1 | 0.5 | 3 |

TABLE 22
Echocardiography: Left ventricular posterior wall [mm] − Treatment completers − LVPW > 9 mm at baseline

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 9 | 0 | 11.2 | 1.0 | 10 | 11.0 | 13 | 0 | 9 | | | | | |
| Treatment | Week 24 | 9 | 0 | 11.0 | 1.0 | 9 | 11.0 | 12 | 9 | 0 | −0.2 | 1.0 | −1 | 0.0 | 2 |
| Baseline | | 6 | 3 | 11.3 | 1.0 | 10 | 11.0 | 13 | 0 | 9 | | | | | |
| Follow up | Week 12 | 6 | 3 | 10.3 | 0.8 | 10 | 10.0 | 12 | 6 | 3 | −1.0 | 1.1 | −3 | −1.0 | 0 |
| Baseline | | 4 | 5 | 11.3 | 1.3 | 10 | 11.0 | 13 | 0 | 9 | | | | | |
| Follow up | Week 24 | 4 | 5 | 11.3 | 1.3 | 10 | 11.0 | 13 | 4 | 5 | 0.0 | 0.0 | 0 | 0.0 | 0 |
| Baseline | | 9 | 0 | 11.2 | 1.0 | 10 | 11.0 | 13 | 0 | 9 | | | | | |
| Last available visit | | 9 | 0 | 11.1 | 1.1 | 10 | 11.0 | 13 | 9 | 0 | −0.1 | 0.9 | −1 | 0.0 | 2 |

8 MIU IFNβ-1b

| | | | | | | | | | | | | | | | |
|---|---|---|---|---|---|---|---|---|---|---|---|---|---|---|---|
| Baseline | | 11 | 1 | 10.7 | 0.8 | 10 | 11.0 | 12 | 0 | 12 | | | | | |
| Treatment | Week 24 | 11 | 1 | 10.6 | 1.3 | 10 | 10.0 | 14 | 11 | 1 | −0.1 | 1.7 | −2 | 0.0 | 4 |
| Baseline | | 8 | 4 | 10.8 | 0.7 | 10 | 11.0 | 12 | 0 | 12 | | | | | |
| Follow up | Week 12 | 8 | 4 | 10.6 | 0.9 | 9 | 11.0 | 12 | 8 | 4 | −0.1 | 1.1 | −2 | 0.0 | 1 |
| Baseline | | 5 | 7 | 11.0 | 1.0 | 10 | 11.0 | 12 | 0 | 12 | | | | | |
| Follow up | Week 24 | 5 | 7 | 10.6 | 0.9 | 10 | 10.0 | 12 | 5 | 7 | −0.4 | 0.5 | −1 | 0.0 | 0 |
| Baseline | | 11 | 1 | 10.7 | 0.8 | 10 | 11.0 | 12 | 0 | 12 | | | | | |
| Last available visit | | 11 | 1 | 10.7 | 0.8 | 10 | 11.0 | 12 | 11 | 1 | 0.0 | 0.8 | −1 | 0.0 | 1 |

Hemodynamic Measurement

Table 23 shows the changes in wedge pressure at rest from baseline to follow-up week 12 after the end of treatment in patients in whom this parameter was assessed both at baseline and after the end of treatment.

In the 4 MIU group the mean of the wedge pressure at rest decreased from 16.0 to 14.3 mmHg compared to baseline. In the 8 MIU group the mean of the wedge pressure rest decreased from 9.4 to 7.4 mmHg compared to baseline.

Table 24 shows the changes in mean pulmonary artery pressure at rest from baseline to follow-up week 12 after the end of treatment in patients in whom this parameter was assessed both at baseline and after the end of treatment.

In the 4 MIU group the mean of the mean pulmonary artery pressure at rest decreased from 24.3 to 20.0 mmHg compared to baseline. In the 8 MIU group the mean of the mean pulmonary pressure at rest decreased from 16.3 to 15.1 mmHg compared to baseline.

Summary

The decrease of wedge pressure at rest and mean pulmonary artery pressure at rest is the expression of an improved left ventricular function, which can be the result of a re-remodelling induced by treatment with IFNβ-1b.

TABLE 23
Wedge pressure at rest [mm Hg] − Treatment completers

| Measured values | | | | | | | | | | | | Changes from baseline | | | | |
|---|---|---|---|---|---|---|---|---|---|---|---|---|---|---|---|---|
| Study phase | Week | Cardiovascular test | N | Nmiss | Mean | SD | Min | Median | Max | N | Nmiss | Mean | SD | Min | Median | Max |

4 MIU IFNβ-1b

| | | | | | | | | | | | | | | | | |
|---|---|---|---|---|---|---|---|---|---|---|---|---|---|---|---|---|
| Baseline | | ergometry, rest | 3 | 11 | 16.0 | 8.9 | 9 | 13.0 | 26 | 0 | 14 | | | | | |
| Follow-up | Week 12 | ergometry, rest | 3 | 11 | 14.3 | 6.5 | 8 | 14.0 | 21 | 3 | 11 | −1.7 | 14.2 | −18 | 5.0 | 8 |

8 MIU IFNβ-1b

| | | | | | | | | | | | | | | | | |
|---|---|---|---|---|---|---|---|---|---|---|---|---|---|---|---|---|
| Baseline | | ergometry, rest | 10 | 8 | 9.4 | 6.1 | 5 | 7.5 | 26 | 0 | 18 | | | | | |
| Follow-up | Week 12 | ergometry, rest | 10 | 8 | 7.4 | 3.7 | 3 | 6.5 | 15 | 10 | 8 | −2.0 | 7.8 | −21 | −1.0 | 8 |

TABLE 24
Mean pulmonary artery pressure at rest [mm Hg] − Treatment completers

| Measured values | | | | | | | | | | | | Changes from baseline | | | | |
|---|---|---|---|---|---|---|---|---|---|---|---|---|---|---|---|---|
| Study phase | Week | Cardiovascular test | N | Nmiss | Mean | SD | Min | Median | Max | N | Nmiss | Mean | SD | Min | Median | Max |

4 MIU IFNβ-1b

| | | | | | | | | | | | | | | | | |
|---|---|---|---|---|---|---|---|---|---|---|---|---|---|---|---|---|
| Baseline | | ergometry, rest | 3 | 11 | 24.3 | 11.8 | 17 | 18.0 | 38 | 0 | 14 | | | | | |
| Follow-up | Week | ergometry, rest | 3 | 11 | 20.0 | 4.6 | 15 | 21.0 | 24 | 3 | 11 | −4.3 | 16.2 | −23 | 4.0 | 6 |
| | 12 | | | | | | | | | | | | | | | |

8 MIU IFNβ-1b

| | | | | | | | | | | | | | | | | |
|---|---|---|---|---|---|---|---|---|---|---|---|---|---|---|---|---|
| Baseline | | ergometry, rest | 12 | 6 | 16.3 | 9.7 | 9 | 12.5 | 43 | 0 | 18 | | | | | |
| Follow-up | Week | ergometry, rest | 12 | 6 | 15.1 | 6.1 | 9 | 13.0 | 30 | 12 | 6 | −1.2 | 9.9 | −30 | 0.0 | 11 |
| | 12 | | | | | | | | | | | | | | | |

Comparison Between Outcome in NYHA Functional Class and Other Parameters

Table 25 shows the change of LVEDD in patients with improvement or no improvement in NYHA functional class after the end of treatment compared to baseline.

In the 4 MIU group, the mean decrease of LVEDD after the end of treatment was more pronounced in patients who showed also an improvement regarding NYHA functional class (−3.3 to −5.8 mm vs −0.7 to −1.2 mm). Similarly, in the 8 MIU group the mean change of LVEDD after the end of treatment in patients who showed also an improvement regarding NYHA functional class was −1.9 to −2.8 compared to −0.6 to −1.1 mm in patients who did not show improvement in NYHA functional class.

Table 26 shows the change of LVESD in patients with improvement or no improvement in NYHA functional class after the end of treatment compared to baseline.

In the 4 MIU group, the mean decrease of LVESD after the end of treatment was −4.7 to −5.7 mm in patients who showed also an improvement regarding NYHA functional class compared to a mean change by 0.8 to −0.8 mm in patients who did not show improvement regarding NYHA functional class. Similarly, in the 8 MIU group the mean change of LVEDD after the end of treatment in patients who showed also an improvement regarding NYHA functional class was −1.8 to −4.3 compared to 0.0 to −1.7 mm in patients who did not show improvement in NYHA functional class.

Table 27 shows the change of wedge pressure at rest in patients with improvement or no improvement in NYHA functional class after the end of treatment compared to baseline.

In the 4 MIU group, the patient who did improve regarding NYHA functional class after the end of treatment showed a wedge pressure at rest decrease from 26 to 8 mmHg whereas the mean wedge pressure at rest changed from 11 to 17.5 mmHg in patients who did not show improvement regarding NYHA functional class. In the 8 MIU group the mean change of wedge pressure at rest decreased from 11.6 to 8.2 mmHg after the end of treatment in patients who showed also an improvement regarding NYHA functional class compared to 7.2 to 6.6 mmHg in patients who did not show improvement in NYHA functional class.

TABLE 25a
Echocardiography: LVEDD [mm] − Treatment completers − NYHA class declined/unchanged

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 5 | 1 | 64.2 | 9.0 | 50 | 65.0 | 74 | 0 | 6 | | | | | |
| Treatment | Week 24 | 5 | 1 | 63.0 | 10.5 | 46 | 68.0 | 72 | 5 | 1 | −1.2 | 4.8 | −5 | −4.0 | 5 |
| Baseline | | 6 | 0 | 66.7 | 10.1 | 50 | 67.0 | 79 | 0 | 6 | | | | | |
| Follow up | Week 12 | 6 | 0 | 66.0 | 8.9 | 50 | 68.5 | 76 | 6 | 0 | −0.7 | 4.1 | −6 | −1.0 | 6 |
| Baseline | | 4 | 2 | 71.3 | 6.8 | 63 | 71.5 | 79 | 0 | 6 | | | | | |
| Follow up | Week 24 | 4 | 2 | 70.5 | 3.1 | 68 | 69.5 | 75 | 4 | 2 | −0.8 | 5.7 | −6 | −2.0 | 7 |

8 MIU IFNβ-1b

| | | | | | | | | | | | | | | | |
|---|---|---|---|---|---|---|---|---|---|---|---|---|---|---|---|
| Baseline | | 10 | 0 | 55.8 | 10.2 | 40 | 55.5 | 71 | 0 | 10 | | | | | |
| Treatment | Week 24 | 10 | 0 | 55.1 | 9.7 | 38 | 54.5 | 70 | 10 | 0 | −0.7 | 2.4 | −4 | −1.0 | 3 |
| Baseline | | 9 | 1 | 56.1 | 10.8 | 40 | 58.0 | 71 | 0 | 10 | | | | | |
| Follow up | Week 12 | 9 | 1 | 55.0 | 11.6 | 37 | 50.0 | 70 | 9 | 1 | −1.1 | 2.9 | −8 | 0.0 | 1 |
| Baseline | | 5 | 5 | 56.2 | 10.6 | 45 | 53.0 | 71 | 0 | 10 | | | | | |
| Follow up | Week 24 | 5 | 5 | 55.6 | 11.6 | 44 | 54.0 | 72 | 5 | 5 | −0.6 | 1.7 | −3 | −1.0 | 1 |

TABLE 25b
Echocardiography: LVEDD [mm] - Treatment completers - NYHA class improved at least one stage

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 5 | 1 | 62.8 | 9.7 | 50 | 66.0 | 74 | 0 | 6 | | | | | |
| Treatment | Week 24 | 5 | 1 | 57.2 | 11.3 | 43 | 64.0 | 68 | 5 | 1 | −5.6 | 2.7 | −9 | −6.0 | −2 |
| Baseline | | 4 | 2 | 71.5 | 5.5 | 66 | 71.0 | 78 | 0 | 6 | | | | | |
| Follow up | Week 12 | 4 | 2 | 65.8 | 5.3 | 59 | 67.0 | 70 | 4 | 2 | −5.8 | 3.3 | −9 | −6.0 | −2 |
| Baseline | | 3 | 3 | 64.0 | 12.5 | 50 | 68.0 | 74 | 0 | 6 | | | | | |
| Follow up | Week 24 | 3 | 3 | 60.7 | 13.7 | 45 | 67.0 | 70 | 3 | 3 | −3.3 | 2.1 | −5 | −4.0 | −1 |

8 MIU IFNβ-1b

| | | | | | | | | | | | | | | | |
|---|---|---|---|---|---|---|---|---|---|---|---|---|---|---|---|
| Baseline | | 7 | 0 | 57.6 | 6.9 | 44 | 59.0 | 64 | 0 | 7 | | | | | |
| Treatment | Week 24 | 7 | 0 | 55.7 | 5.8 | 46 | 57.0 | 63 | 7 | 0 | −1.9 | 3.0 | −6 | −2.0 | 2 |
| Baseline | | 4 | 3 | 56.8 | 9.2 | 44 | 59.5 | 64 | 0 | 7 | | | | | |
| Follow up | Week 12 | 4 | 3 | 54.0 | 5.7 | 46 | 56.0 | 58 | 4 | 3 | −2.8 | 3.6 | −6 | −3.5 | 2 |
| Baseline | | 4 | 3 | 59.3 | 4.4 | 55 | 59.0 | 64 | 0 | 7 | | | | | |
| Follow up | Week 24 | 4 | 3 | 56.8 | 5.5 | 52 | 56.5 | 62 | 4 | 3 | −2.5 | 1.3 | −4 | −2.5 | −1 |

TABLE 26a
Echocardiography: LVESD [mm] - Treatment completers - NYHA class declined/unchanged

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 5 | 1 | 48.6 | 9.2 | 33 | 53.0 | 56 | 0 | 6 | | | | | |
| Treatment | Week 24 | 5 | 1 | 48.4 | 14.2 | 27 | 55.0 | 60 | 5 | 1 | −0.2 | 6.5 | −7 | −1.0 | 7 |
| Baseline | | 5 | 1 | 51.0 | 12.5 | 33 | 53.0 | 68 | 0 | 6 | | | | | |
| Follow up | Week 12 | 5 | 1 | 50.2 | 15.1 | 28 | 55.0 | 67 | 5 | 1 | −0.8 | 4.4 | −5 | −1.0 | 5 |
| Baseline | | 4 | 2 | 57.5 | 7.1 | 53 | 54.5 | 68 | 0 | 6 | | | | | |
| Follow up | Week 24 | 4 | 2 | 58.3 | 6.6 | 54 | 55.5 | 68 | 4 | 2 | 0.8 | 1.7 | −1 | 0.5 | 3 |

8 MIU IFNβ-1b

| | | | | | | | | | | | | | | | |
|---|---|---|---|---|---|---|---|---|---|---|---|---|---|---|---|
| Baseline | | 9 | 0 | 40.9 | 11.8 | 21 | 47.0 | 54 | 0 | 9 | | | | | |
| Treatment | Week 24 | 9 | 0 | 39.2 | 12.0 | 19 | 40.0 | 59 | 9 | 0 | −1.7 | 3.9 | −8 | −1.0 | 5 |
| Baseline | | 8 | 1 | 41.4 | 12.5 | 21 | 47.5 | 54 | 0 | 9 | | | | | |
| Follow up | Week 12 | 8 | 1 | 40.4 | 13.3 | 18 | 43.0 | 55 | 8 | 1 | −1.0 | 4.2 | −8 | −0.5 | 5 |
| Baseline | | 5 | 4 | 40.6 | 11.9 | 27 | 37.0 | 54 | 0 | 9 | | | | | |
| Follow up | Week 24 | 5 | 4 | 40.6 | 12.2 | 25 | 39.0 | 56 | 5 | 4 | 0.0 | 2.3 | −3 | 1.0 | 2 |

TABLE 26b
Echocardiography: LVESD [mm] - Treatment completers - NYHA class improved at least one stage

| Measured values | | | | | | | | | | | Changes from baseline | | | | |
|---|---|---|---|---|---|---|---|---|---|---|---|---|---|---|---|
| Study phase | Week | N | Nmiss | Mean | SD | Min | Median | Max | N | Nmiss | Mean | SD | Min | Median | Max |

4 MIU IFNβ-1b

| | | | | | | | | | | | | | | | |
|---|---|---|---|---|---|---|---|---|---|---|---|---|---|---|---|
| Baseline | | 3 | 3 | 41.7 | 12.3 | 28 | 45.0 | 52 | 0 | 6 | | | | | |
| Treatment | Week 24 | 3 | 3 | 36.0 | 13.1 | 22 | 38.0 | 48 | 3 | 3 | −5.7 | 1.5 | −7 | −6.0 | −4 |
| Baseline | | 3 | 3 | 54.0 | 5.3 | 50 | 52.0 | 60 | 0 | 6 | | | | | |
| Follow up | Week 12 | 3 | 3 | 49.0 | 11.0 | 38 | 49.0 | 60 | 3 | 3 | −5.0 | 7.8 | −14 | −1.0 | 0 |
| Baseline | | 3 | 3 | 47.7 | 17.9 | 28 | 52.0 | 63 | 0 | 6 | | | | | |
| Follow up | Week 24 | 3 | 3 | 43.0 | 14.9 | 26 | 49.0 | 54 | 3 | 3 | −4.7 | 3.8 | −9 | −3.0 | −2 |

8 MIU IFNβ-1b

| | | | | | | | | | | | | | | | |
|---|---|---|---|---|---|---|---|---|---|---|---|---|---|---|---|
| Baseline | | 6 | 1 | 42.5 | 9.7 | 26 | 44.0 | 55 | 0 | 7 | | | | | |
| Treatment | Week 24 | 6 | 1 | 40.7 | 7.3 | 29 | 41.0 | 49 | 6 | 1 | −1.8 | 6.5 | −12 | −1.0 | 6 |
| Baseline | | 3 | 4 | 42.0 | 14.7 | 26 | 45.0 | 55 | 0 | 7 | | | | | |
| Follow up | Week 12 | 3 | 4 | 37.7 | 8.7 | 28 | 40.0 | 45 | 3 | 4 | −4.3 | 6.0 | −10 | −5.0 | 2 |
| Baseline | | 4 | 3 | 45.5 | 6.8 | 39 | 44.0 | 55 | 0 | 7 | | | | | |
| Follow up | Week 24 | 4 | 3 | 41.8 | 7.3 | 36 | 39.5 | 52 | 4 | 3 | −3.8 | 3.6 | −9 | −2.5 | −1 |

TABLE 27a
Wedge pressure at rest [mm Hg] - Treatment completers - NYHA class declined/unchanged

| Measured values | | | | | | | | | | | | Changes from baseline | | | | |
|---|---|---|---|---|---|---|---|---|---|---|---|---|---|---|---|---|
| Study phase | Week | Cardiovascular test | N | Nmiss | Mean | SD | Min | Median | Max | N | Nmiss | Mean | SD | Min | Median | Max |

4 MIU IFNβ-1b

| | | | | | | | | | | | | | | | | |
|---|---|---|---|---|---|---|---|---|---|---|---|---|---|---|---|---|
| Baseline | | ergometry, rest | 2 | 4 | 11.0 | 2.8 | 9 | 11.0 | 13 | 0 | 6 | | | | | |
| Follow-up | Week 12 | ergometry, rest | 2 | 4 | 17.5 | 4.9 | 14 | 17.5 | 21 | 2 | 4 | 6.5 | 2.1 | 5 | 6.5 | 8 |

8 MIU IFNβ-1b

| | | | | | | | | | | | | | | | | |
|---|---|---|---|---|---|---|---|---|---|---|---|---|---|---|---|---|
| Baseline | | ergometry, rest | 5 | 5 | 7.2 | 2.0 | 5 | 8.0 | 9 | 0 | 10 | | | | | |
| Follow-up | Week 12 | ergometry, rest | 5 | 5 | 6.6 | 3.4 | 3 | 6.0 | 12 | 5 | 5 | −0.6 | 3.4 | −4 | −2.0 | 4 |

TABLE 27b
Wedge pressure at rest [mm Hg] - Treatment completers - NYHA class improved at least one stage

| Measured values | | | | | | | | | | | | Changes from baseline | | | | |
|---|---|---|---|---|---|---|---|---|---|---|---|---|---|---|---|---|
| Study phase | Week | Cardiovascular test | N | Nmiss | Mean | SD | Min | Median | Max | N | Nmiss | Mean | SD | Min | Median | Max |

4 MIU IFNβ-1b

| | | | | | | | | | | | | | | |
|---|---|---|---|---|---|---|---|---|---|---|---|---|---|---|
| Baseline | | ergometry, rest | 1 | 5 | 26.0 | 26 | 26.0 | 26 | 0 | 6 | | | | |
| Follow-up | Week 12 | ergometry, rest | 1 | 5 | 8.0 | 8 | 8.0 | 8 | 1 | 5 | −18.0 | −18 | −18.0 | −18 |

8 MIU IFNβ-1b

| | | | | | | | | | | | | | | | | |
|---|---|---|---|---|---|---|---|---|---|---|---|---|---|---|---|---|
| Baseline | | ergometry, rest | 5 | 2 | 11.6 | 8.2 | 7 | 7.0 | 26 | 0 | 7 | | | | | |
| Follow-up | Week 12 | ergometry, rest | 5 | 2 | 8.2 | 4.1 | 5 | 7.0 | 15 | 5 | 2 | −3.4 | 11.0 | −21 | 0.0 | 8 |

TABLE 28a
Pulmonary artery pressure at rest [mm Hg] - Treatment completers - NYHA class declined/unchanged

| Measured values | | | | | | | | | | | | Changes from baseline | | | | |
|---|---|---|---|---|---|---|---|---|---|---|---|---|---|---|---|---|
| Study phase | Week | Cardiovascular test | N | Nmiss | Mean | SD | Min | Median | Max | N | Nmiss | Mean | SD | Min | Median | Max |

4 MIU IFNβ-1b

| | | | | | | | | | | | | | | | | |
|---|---|---|---|---|---|---|---|---|---|---|---|---|---|---|---|---|
| Baseline | | ergometry, rest | 2 | 4 | 17.5 | 0.7 | 17 | 17.5 | 18 | 0 | 6 | | | | | |
| Follow-up | Week 12 | ergometry, rest | 2 | 4 | 22.5 | 2.1 | 21 | 22.5 | 24 | 2 | 4 | 5.0 | 1.4 | 4 | 5.0 | 6 |

8 MIU IFNβ-1b

| | | | | | | | | | | | | | | | | |
|---|---|---|---|---|---|---|---|---|---|---|---|---|---|---|---|---|
| Baseline | | ergometry, rest | 6 | 4 | 12.7 | 0.8 | 12 | 12.5 | 14 | 0 | 10 | | | | | |
| Follow-up | Week 12 | ergometry, rest | 6 | 4 | 13.3 | 5.4 | 9 | 11.5 | 24 | 6 | 4 | 0.7 | 5.2 | −3 | −1.0 | 11 |

TABLE 28b
Mean pulmonary artery pressure at rest [mm Hg] - Treatment completers - NYHA class improved at least one stage

| Measured values | | | | | | | | | | | | Changes from baseline | | | | |
|---|---|---|---|---|---|---|---|---|---|---|---|---|---|---|---|---|
| Study phase | Week | Cardiovascular test | N | Nmiss | Mean | SD | Min | Median | Max | N | Nmiss | Mean | SD | Min | Median | Max |

4 MIU IFNβ-1b

| | | | | | | | | | | | | | | |
|---|---|---|---|---|---|---|---|---|---|---|---|---|---|---|
| Baseline | | ergometry, rest | 1 | 5 | 38.0 | 38 | 38.0 | 38 | 0 | 6 | | | | |
| Follow-up | Week | ergometry, rest | 1 | 5 | 15.0 | 15 | 15.0 | 15 | 1 | 5 | −23.0 | −23 | −23.0 | −23 |
| | 12 | | | | | | | | | | | | | |

8 MIU IFNβ-1b

| | | | | | | | | | | | | | | | | |
|---|---|---|---|---|---|---|---|---|---|---|---|---|---|---|---|---|
| Baseline | | ergometry, rest | 6 | 1 | 19.8 | 13.2 | 9 | 14.0 | 43 | 0 | 7 | | | | | |
| Follow-up | Week | ergometry, rest | 6 | 1 | 16.8 | 6.7 | 11 | 15.5 | 30 | 6 | 1 | −3.0 | 13.4 | −30 | 1.0 | 6 |
| | 12 | | | | | | | | | | | | | | | |

Table 28 shows the change of mean pulmonary artery pressure at rest in patients with improvement or no improvement in NYHA functional class after the end of treatment compared to baseline. In the 4 MIU group, the patient who did improve regarding NYHA functional class after the end of treatment showed a mean pulmonary artery pressure at rest decrease from 38 to 15 mmHg whereas the mean of the mean pulmonary artery pressure at rest changed from 17.5 to 22.5 mmHg in patients who did not show improvement regarding NYHA functional class. In the 8 MIU group the mean of the mean pulmonary artery pressure at rest decreased from 19.8 to 16.8 mmHg after the end of treatment in patients who showed also an improvement regarding NYHA functional class compared to 12.7 to 13.3 mmHg in patients who did not show improvement in NYHA functional class.

Summary

The changes in LVEDD and LVESD show that there was a markedly decrease of both left ventricular diameters in patients who also improved regarding NYHA functional class after the end of treatment. On the other hand the mean LVEDD and mean LVESD remained almost unchanged or decreased only slightly in patients who did show no improvement in NYHA functional class. It can be concluded that normalization of the left ventricle probably induced by treatment with IFNβ-1b is connected with improvement in clinical outcome.

The changes in mean wedge pressure at rest and the mean of the mean pulmonary artery pressure at rest in the 4 MIU group show that there was a markedly decrease of both mean values in patients who also improved regarding NYHA functional class after the end of treatment. On the other hand the mean wedge pressure at rest and the mean of the pulmonary artery at rest increased slightly in patients of the 4 MIU group who did show no improvement in NYHA functional class. In the 8 MIU group the mean wedge pressure at rest decreased after the end of treatment regardless whether the patients did also show improvement in NYHA functional class.

The decrease of wedge pressure at rest and mean pulmonary artery pressure at rest is the expression of an improved left ventricular function, which can be the result of a re-remodelling induced by treatment with IFNβ-1b. The decrease in wedge pressure at rest and mean pulmonary artery pressure at rest seems to be connected to the improvement in clinical outcome. The effect of 8 MIU IFNβ-1b per injection seems to be more pronounced since the mean wedge pressure at rest decreases also in patients who did show no improvement in NYHA functional class.

Conclusion

The interim results of this example show that there are favourable results regarding absence or reduction of Parvovirus B19 load, and absence of HHV6 in the endomyocardial biopsy, clinical outcome, echocardiographic parameters, LVEF and hemodynamic parameters in patients who were treated with IFNβ-1b. The benefical effects on virus elimination, normalization of left ventricular diameters, LVEF increase and reduction of wedge pressure at rest and mean pulmonary artery pressure at rest, improvement in clinical outcome seem to be induced by treatment with IFNβ-1b. These beneficial effects regarding virus elimination and/or virus load reduction seem to be more pronounced in patients who were treated with 8 MIU IFNβ-1b/injection compared to patients who were treated with 4 MIU IFNβ-1b/injection.

TABLE 29
Comparison to 29 pts with Adenovirus/Enterovirus of the study described in
Example 1 to patients of example 2.

| | | EXAMPLE 2 | EXAMPLE 2 |
|---|---|---|---|
| | | (Parvovirus/HHV6 | (Parvovirus/HHV6 |
| PARAMETER | EXAMPLE 1 | PTS) 4 MIU group | PTS) 8 MIU group |
| LVEDD | Slight decrease | Mean decrease by −1.9 | Mean decrease by −1.2 |
| | | to −2.8 mm | to −2.6 mm |
| LVESD | Slight decrease | Mean decrease by −1.6 | Mean decrease by −1.7 |
| | | to −2.4 mm | to −1.9 mm |
| LVEF at rest | Mean increased from | Median increase by | Median increase by |
| | 44.3 to 48.3% | 3.5% | 4.5% |
| Wedge pressure at | n.d. | Mean decreased from | Mean decreased from |
| rest | | 16.0 to 14.3 mmHg | from 9.4 to 7.4 mmHg |
| Mean pulmonary | n.d. | Mean decreased from | Mean decreased from |
| artery pressure at rest | | 24.3 to 20.0 mmHg | 16.3 to 15.1 |
| NYHA functional class | Improved in 75% of pts | Improved in 50.0% of | Improved in 41.1% of |
| | | pts | pts |
| Virus elimination | Elimination of | Elimination or | Elimination or |
| | Enterovirus, | reduction of Parvovirus | reduction of Parvovirus |
| | Adenovirus | B19 load in 20% of pts | B19 load in 46.6% of |
| | | Elimination of HHV6 | pts |
| | | in 33% of pts | Elimination of HHV6 |
| | | | in 85.7% of pts |
| Quality of life | n.d. | Improved in 60% of pts | Improved in 60% of pts |
| Patient global | n.d. | Improved in 80% of | Improved in 82.3% of |
| assessment | | pts | pts |
| Composite clinical end | n.d. | Improved in 66.7% of | improved in 64.7% of |
| point | | pts | pts |

### Example 3

### Treatment of Cardiomyopathy using IFNβ-1b

This example describes a study for determining and carrying out the safe and efficacious treatment of chronic viral cardiomyopathy using a therapeutically effective dose of IFNβ-1b.

Summary

The safe and efficacious use of IFNβ-1b (4 and 8 MIU) in the treatment of patients with chronic viral cardiomyopathy (e.g., Adenovirus, Enterovirus and Parvovirus) can be evaluated and carried out as described herein. More particularly, this study, including the treatment and follow-up schedule as well as the efficacy and safety parameters can be determined and carried out as described herein including e.g., as described in Examples 1 and 2 (above).

Method and Study Design (Table 30)

TABLE 30
Methods and study design for a double-blind, placebo-controlled, randomized, parallel
group, multicenter study to evaluate efficacy and safety of 4 and 8 million units
Betaferon ®/Betaseron ® (IFNβ-1b) given subcutaneously every other
day over 24 weeks in patients with chronic viral cardiomyopathy.

| Study phase | Phase II |
|---|---|
| Investigational product, dosage, | Betaferon ®/Betaseron ® (IFNβ-1b), sterile |
| and route of administration | vials with lyophilisate and vials with diluent; |
| | every |
| | other day subcutaneous (s.c.) application |
| | group 1: 2 MIU per application in week 1, |
| | 4 MIU per application in weeks 2 to 24; |
| | group 2: 2 MIU per application in week 1, 4 |
| | MIU per application in weeks 2 to 3, 8 MIU |
| | per application in weeks 4 to 24. |
| Reference product, dosage, | Placebo in sterile vials with lyophilisate and |
| and route of administration | vials with diluent; every other day |
| | subcutaneous (s.c.) application; one half of |
| | the group: 0.25 ml per application in |
| | week 1, 0.5 ml per application in weeks 2 to |
| | 24; the other half of the group: 0.25 ml |
| | per application in week 1, 0.5 ml per |
| | application in weeks 2 to 3, 1.0 ml per |
| | application in weeks 4 to 24. |
| Indication | Chronic viral cardiomyopathy (CVC) |
| Study objective | Evaluation of efficacy and safety; primary |
| | efficacy variable presence of Adeno- |
| | and/or Enterovirus or Parvovirus (without |
| | Adeno-/Enterovirus) 12 weeks ± 14 days |
| | after the end of treatment. |
| Patient population | Patients aged 18 to 75 with biopsy-proven |
| | CVC, showing impaired LV function as |
| | evaluated by radionuclide ventriculography |
| | (RNV) (30% ≦ EF ≦ 55%) or impaired |
| | LV function as evaluated by RNV (25% ≦ EF < 30%) |
| | and wedge pressure < 20 mm |
| | Hg or typical symptoms and signs of heart |
| | failure and preserved left ventricular |
| | systolic function and no valvular |
| | abnormalities on echocardiography and |
| | NYHA II |
| | to III; onset of clinical symptoms at least 6 |
| | months prior to enrollment in the study, |
| | no relevant changes of medication of |
| | chronic heart failure within 8 weeks prior to |
| | enrollment in the study as documented by |
| | medical records. |
| Study Design | Randomized, partially double-blind, |
| | placebo-controlled, stratified, parallel group, |
| | multicenter study |
| Concurrent control | Placebo |
| Duration of treatment | 24 weeks |
| Methodology | Determination of virus in endomyocardial |
| | biopsies by polymerase chain |
| | reaction (PCR). |
| Number of study centers | Approximately 35 in 8 countries |
| Total number of patients, minimum and | Sample size 120-150; minimum 1, |
| Sample size 120-15090 (at least 60 patients | maximum 15 patients per center; the |
| per stratum) (20-30 per group per | sample size is based |
| Maximum number of patients per center, | on a statistical rationale for the primary |
| statistical rationale provided | efficacy variable as well as explorative |
| | assessment of clinical efficacy and safety |
| | variables for dose selection, |
| | approximately 400 patients will have to be |
| | screened |
| Adverse events | As reported spontaneously by the patient |
| | and as evaluated by laboratory |
| | measurements, physical examinations, |
| | echocardiography, and |
| | electrocardiography. AEs will be |
| | continuously reviewed by an independent |
| | data monitoring |
| | committee (IDMC) |
| Plan for data analysis | Stratified Cochran-Armitage trend test on |
| | virus elimination within each stratum, |
| | explorative |
| | analysis of clinical efficacy and safety |
| | variables. |

### Example 4

### Anti-Viral and Cardiomyocyte Protective Effects of Murine Interferon Alpha-2 (IFNα-2) and Murine Interferon Beta (IFNβ) in Coxsackievirus B3-induced Myocarditis in Balb/c Mice

Introduction

It is known that interferons have both anti-viral and immunomodulatory actions. In addition, there is evidence that coxsackievirus B3 (CVB3) is associated with myocarditis and inflammatory cardiomyopathy. Therefore, in the present study, we tested the hypothesis that murine IFNα-2 and murine IFNβ can eliminate cardiac viral load and protect cardiomyocyte from damage in animals infected with CVB3.

Experimental Design

Six-month old Balb/c mice were randomized for intraperitoneal injection of vehicle, murine IFNβ (2.5, 5,10 million international units (MIU)/kg), or murine IFNα-2 (10 MIU/kg). One hour later, these mice were inoculated intraperitonealy with 30,000 PFU of heart-passaged coxsackievirus B3 (CVB3) H3 strain (day 0). Vehicle or interferon treatment was repeated as subcutaneous injections every other day until the day of sacrifice (day 4, 7 or 14). Naive animals did not receive any treatment. Cardiac viral load was measured by a plaque-forming assay. Myocarditis was evaluated by a histopathological examination of cross-sectional slices of the heart.

Results

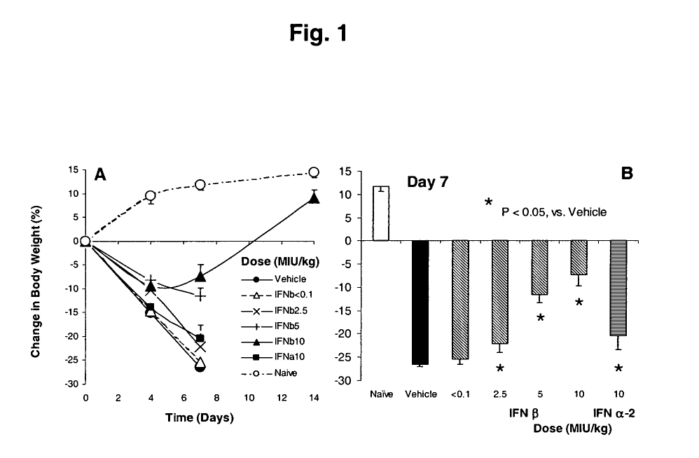

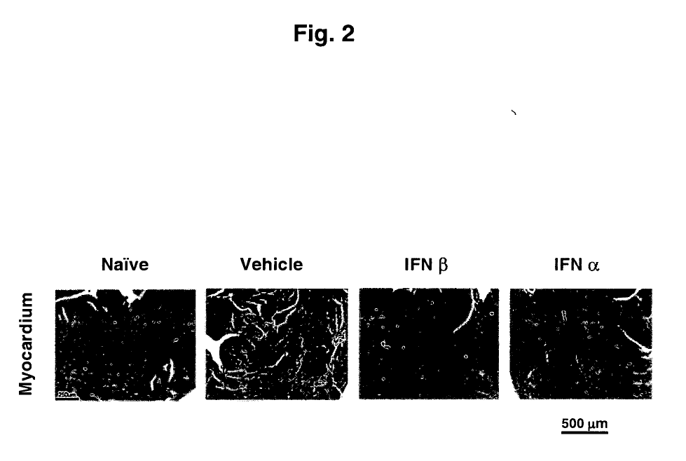

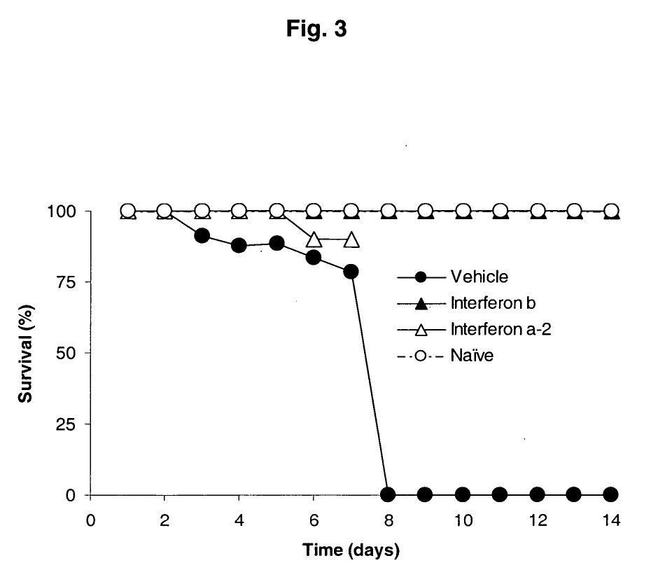

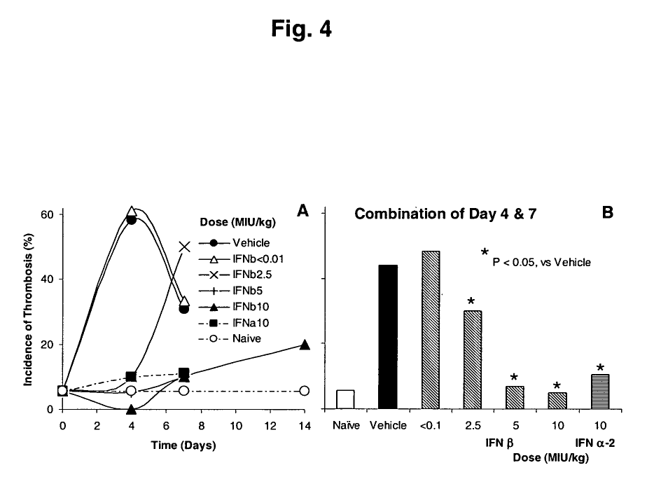

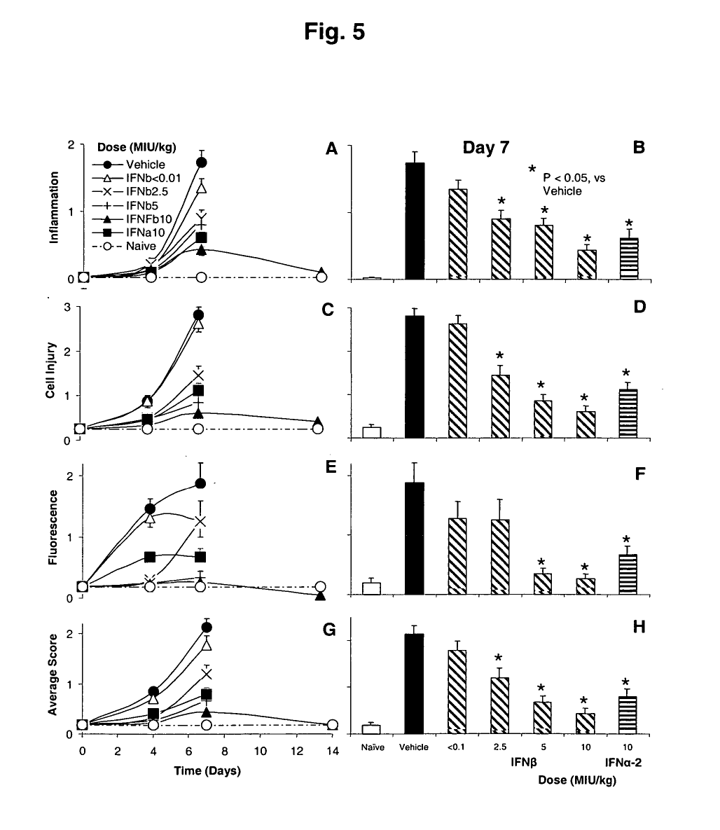

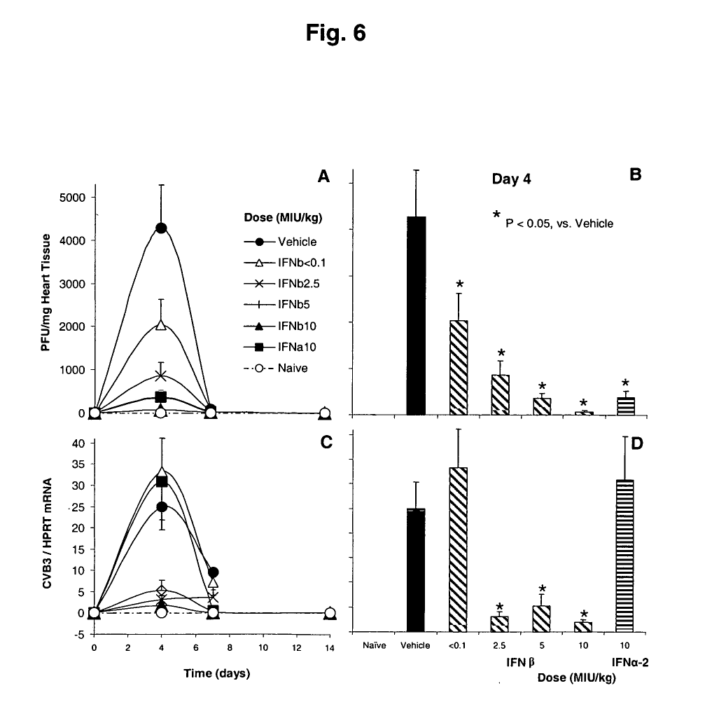

CVB3-inoculated Balb/c mice showed an obvious sick appearance, lethargy, progressive weight loss (FIG. 1), and death (10% on day 3 and 100% on day 8) (FIG. 3). Cardiac viral load dramatically increased (PFU=4277±1009) on day 4 (FIG. 6). Many mice (58%) appeared to have intraventricular thrombi present (FIG. 4) In addition, we observed a progressive inflammatory infiltration and myocyte injury in the heart (FIGS. 2 and 5).

Treatment with a murine IFNβ dose-dependently improved the general health of the CVB3-inoculated mice, reduced weight loss (FIG. 1), prevented death (FIG. 3), eliminated cardiac viral load (FIG. 6), attenuated intraventricular thrombosis formation (FIG. 4), and protected the heart from inflammation and myocyte injury (FIGS. 2 and 5). At the dose of 10 MIU/kg, viral load was reduced by 97% on day 4, incidence of intraventricular thrombosis was reduced to 5%, and the cardiac inflammation and myocyte injury were dramatically reduced on day 7 (FIGS. 6, 4, 2, and 5). While all animals without treatment died on day 8, all those treated with 10 MIU/kg murine IFNβ were healthy, survived to day 14 (FIG. 3) and were free from myocarditis (FIGS. 2 and 4) with an undetectable viral load (FIG. 6).

Treatment with 10 MIU murine IFNα-2 significantly improved the general health in CVB3-treated mice, reduced cardiac viral PFU (374±146), incidence of intraventricular thrombosis (11%), and myocarditis (FIGS. 1, 2, 6, 4, and 5) to a level similar to that achieved with 5 MIU/kg murine IFNβ.

Conclusions

Murine murine IFNβ (2.5 to 10 MIU/kg) dose-dependently prevented CVB3-induced myocarditis with elimination of cardiac viral load, attenuation of intraventricular thrombosis formation, protection of the heart from inflammation and myocyte injury, improvement of general health appearance, prevention of death in Balb/c mice. Murine IFNα-2 at 10 MIU/kg resulted in a similar therapeutic effect, however, at a level equivalent to that achieved by 5 MIU/kg murine IFNβ.

Synopsis of Medical Findings from Examples 1 to 4

Example 1 focuses on beneficial treatment effects of IFNβ in cardiomyopathy patients who have a defined presence of enteroviral or adenoviral genome in the heart tissue. The results show complete elimination of these virus from heart tissue specimen and related clinical improvements. The majority of patients had a relevant improvement in their clinical status and variables of cardiac function. The apparent corelation of clinical improvements with absence of viral genome in the heart appears to support a causality that persistence of viral genome was the only or dominant pathogenic factor. It is, however, likely that a dual mechanism of action of the IFNβ therapy leads to clinical improvement, i.e. there can be virus elimination independent of improved heart function or improved heart function without a relevant degree of virus elimination. There were patients who did not improve despite absence of viral genome. This may be indicative of either disease factors influencing the ability of reversal of cardiac remodeling or may be indicative of therapeutic effects of the IFNβ treatment that are mediated by other mechanisms than the antiviral effects. The rationale for selecting this particular subgroup of patients was in part due to a high likelihood of reaching a treatment benefit due to antiviral and immunomodulatory effects of beta interferon. Incidential observations of patients reducing or eliminating also other virus found in the myocardial tissue led to the further assessments described in Example 2.

Example 2 excluded patients with evidence of enteroviral or adenoviral genome in the myocardium and focussed on the patients with cardiomyopathy and presence of two other virus of which reports of associated myocardial disease was available, i.e. the human parvovirus B19 and the human Herpesvirus type 6. Parvovirus B19 can lead to cardiac functional and structural impairment more by reactive inflammatory mechanisms than the enteroviral infection. In addition the presence of parvovirus B19 in low amounts may not necessarily cause cardiac disease, whereas higher amounts of parvovirus genome in the heart are thought to be associated with cardiac disease. The human Herpesvirus Type 6 can either play a facilitating role, be pathogenic on its own, or have no contributing pathogenic effect when its genome is identified in the cardiac tissue. (see e.g., Bultmann et al. (2003) Virchows Arch. 442: 8-17; Dettmeyer et al. (2003) J Forensic Sci. 48: 183-6; Kandolf et al. (2004) Dtsch Med Wochenschr. 129: 2187-92; Tschöpe et al. (2005) Circulation. 111: 879-86; Kuhl et al. (2005) Circulation. 111: 887-93).

Example 2 shows that the parvoviral genome was frequently found reduced or absent in endomyocardial biopsies after IFNβ-1b therapy as a dose dependent effect. Similarly, patients who had human Herpesvirus Type 6 genome findings were tested negative for this virus after therapy. A majority of patients showed cardiac functional improvements after the IFNβ therapy. Again, this may be indicative of either of disease factors influencing the ability of reversal of cardiac remodeling or may be indicative of therapeutic effects of the IFNβ treatment that are mediated by other mechanisms than the antiviral effects. Such other, immunomodulatory mechanisms leading to a restitution of cardiac function and clinical improvement would then also be considered relevant for other forms of cardiomyopathy, if the contribution of one or the other of the assessed virus as a cardiac pathogenic agent is of lesser importance in these patients.

Example 3 is an ongoing study that assesses these effects of IFNβ therapy in a placebo controlled design. The study includes patients who will show evidence of viral genome of enterovirus, adenovirus, human parvovirus B19 in their endomyocardial biopsies and human Herpesvirus type 6 will also be assessed. It is expected to provide additional information on the absence or reduction of viral genome and associated clinical improvements and improvements of cardiac function. Furthermore, information from this study is expected to contribute to assessing the extent of relevance of the quantitated finding of parvoviral genome in the endomyocardial biopsy for effects of the therapy with IFNβ.

Example 4 is an animal study in a murine model of human myocarditis and viral cardiomyopathy. The model starts with an acute phase characterized by high virus replication and increasing tissue damage that is followed by a chronic phase, characterized by lower virus replication and viral presence and growing tissue damage due to secondary inflammatory effects. The model reflects adequately antiviral effects of the interferon in the target tissue which are part of the mechanism of action in the chronic human viral cardiomyopathy. Human chronic viral cardiomyopathy would also have signals of viral activity and replication in the heart. In this respect, data from the acute phase of the model would represent a stage of the human chronic condition. Human chronic viral cardiomyopathy shows pathomechanisms similar to processes that the model mimicks with the coxsackievirus induced disease Virus elimination was demonstrated over several doses and was due to interferon effects. The results show efficacy of IFNα as well as IFNβ and also show that the lower doses of IFNβ or the lower efficacy of IFNα, translate to lower beneficial effects on cardiac function, as seen in the number of deaths and weight loss, as well as inflammatory findings.

The course of the high dose group with IFNβ compared with the other doses and controls seems to point at a dual mechanism of action in this disease. The antiviral effect has led to virtual absence of replicating virus on days 4 and 7 and a very low level of viral genome detected by PCR on day 4 and absence of viral genome on day 7 (see FIG. 6). Cardiac inflammation was nevertheless measurable, had a peak on day 7 and completely normalized under continued IFNβ therapy until day 14 (see FIG. 5). Therefore, also the beneficial effects of IFN therapy that may be independent of the antiviral processes are a factor leading to the study results. Beneficial effects of the IFN therapy that are independent of antiviral effects are also expected to be an element in treating the human cardiomyopathy.

Cardiac cavity thrombi were also reduced dose-dependently (see FIG. 4) The presence of thrombi could point at an imbalance of prothrombotic versus antithrombotic factors associated with inflammatory induced vascular wall thrombogenicity as an endothelial function impairment usually associated with other markers of endothelial dysfunction. A contribution of rhythm disorders, which also favor cardiac cavity thrombi, cannot be excluded. Cardiac rhythm disorders are ususally a symptom of myocarditis or cardiomyopathy.

The short time course of the acute phase in this murine model constitutes a difference between this murine myocarditis model and the human chronic viral cardiomyopathy. The cardiac damage induced by virus and viral proteins in the mouse heart is at a maximum during the initial acute phase whereas in the human chronic condition one would expect a low virus replication rate, less cell injury, few symptoms of systemic viral disease and a stabilization of cardiac pathology due to repair or other endogenous mechanisms. Nevertheless the virus induced damages are very similar. Therefore the model is adequate to predict effects, particularly in terms of dose response principles, for the human chronic condition. This is supported by the description of the total model, including the chronic phase. The chronic phase of this murine model would be characterized by a lower virus replication rate and higher endogenous immune response and a stabilization at a state of cardiomyopathy. Notably, the mechanisms by which IFN interferes with the disease are in principle the same in either phase of the model.

In summary, results of the human studies show improvements of the cardiomyopathy and a dose dependent antiviral effect in patients. Due to the multiple effects of a IFNβ therapy results may be due to antiviral action that affects disease factors influencing the ability of reversal of cardiac remodeling in patients with cardiomyopathy and at the same time this hints at therapeutic effects of the IFNβ treatment that are mediated by other mechanisms than the antiviral effects. Such mechanisms would, for example, include improvement of endothelial dysfunction. The efficacy of the dual mode of action of IFNβ in this disease is supported by the findings in the animal model in which the dose dependent antiviral effect was conformed over a wider range of doses, the cardiac inflammation and survival as well as the general animal condition was improved and a decrease of disease induced thrombus formation was observed.

1. A pharmaceutical composition having interferon beta (IFNβ) activity and comprising a therapeutically effective amount of an isolated IFNβ or IFNβ mutein for treatment of cardiomyopathy and of endothelial dysfunction,
wherein said therapeutically effective amount is in a range from about 30 mcg to about 500 mcg.

2. The pharmaceutical composition according to claim 1, wherein said therapeutically effective amount is about 30 mcg.

3. The pharmaceutical composition according to claim 1, wherein said therapeutically effective amount is about 250 mcg.

4. The pharmaceutical composition according to claim 1, wherein said therapeutically effective amount is about 500 mcg.

5. The pharmaceutical composition according to claim 1, wherein said IFNβ is IFNβ-1a.

6. The pharmaceutical composition according to claim 1, wherein said IFNβ mutein has a cysteine at position 17 deleted or replaced by a neutral amino acid.

7. The pharmaceutical composition according to claim 6, wherein said neutral amino acid is selected from a group consisting of serine, threonine, glycine, alanine, valine, leucine, isoleucine, histidine, tyrosine, phenylalanine, tryptophan, and methionine.

8. The pharmaceutical composition according to claim 7, wherein said neutral amino acid is serine.

9. The pharmaceutical composition according to claim 1, wherein said IFNβ mutein lacks an N-terminal methionine.

10. The pharmaceutical composition according to claim 1, wherein said IFNβ mutein is IFNβ-1b.

11. The pharmaceutical composition according to claim 1, wherein said IFNβ mutein is Betaseron®.

12. The pharmaceutical composition according to claim 1, wherein said pharmaceutical composition is a stabilized, human serum albumin-free (HSA-free) pharmaceutical composition.

13. The pharmaceutical composition according to claim 1, wherein said IFNβ or IFNβ mutein is substantially monomeric and solubilized in a low-ionic-strength formulation.

14. The pharmaceutical composition according to claim 1, wherein said low-ionic-strength formulation is a solution having a pH from about 2 to about 5, and an ionic strength from about 1 to about 100 mM.

15. The pharmaceutical composition according to any one of claims 1-14, wherein said IFNβ or said IFNβ mutein is PEGylated.

16. The pharmaceutical composition according to any one of claims 1-14, wherein said IFNβ is a human IFNβ.

17. The pharmaceutical composition according to any one of claims 1-14, wherein said IFNβ mutein is a human IFNβ mutein.

18. A pharmaceutical composition having interferon alpha (IFNα) activity and comprising a therapeutically effective amount of an isolated IFNα or IFNα mutein for treatment of cardiomyopathy and of endothelial dysfunction,
wherein said therapeutically effective amount is in a range from about 30 mcg to about 500 mcg.

19. The pharmaceutical composition according to claim 18, wherein said therapeutically effective amount is about 30 mcg.

20. The pharmaceutical composition according to claim 18, wherein said therapeutically effective amount is about 250 mcg.

21. The pharmaceutical composition according to claim 18, wherein said therapeutically effective amount is about 500 mcg.

22. The pharmaceutical composition according to claim 18, wherein said pharmaceutical composition is a stabilized, human serum albumin-free (HSA-free) pharmaceutical composition.

23. The pharmaceutical composition according to claim 18, wherein said IFNα or IFNα mutein is substantially monomeric and solubilized in a low-ionic-strength formulation.

24. The pharmaceutical composition according to claim 18, wherein said low-ionic-strength formulation is a solution having a pH from about 2 to about 5, and an ionic strength from about 1 to about 100 mM.

25. The pharmaceutical composition according to any one of claims 18-24, wherein said IFNα or said IFNα mutein is PEGylated.

26. The pharmaceutical composition according to any one of claims 18-24, wherein said IFNα is a human IFNα.

27. The pharmaceutical composition according to any one of claims 18-24, wherein said IFNα mutein is a human IFNα mutein.

28. A method of treating a patient for cardiomyopathy and for endothelial dysfunction comprising administering to said patient the pharmaceutical composition according to any one of claims 1-14 and 18-24.

29. The method according to claim 28, wherein said IFNβ is a human IFNβ.

30. The method according to claim 28, wherein said IFNα mutein is a human IFNβ mutein.

31. The method according to claim 28, wherein said IFNα is a human IFNα.

32. The method according to claim 20, wherein said IFNα mutein is a human IFNα. mutein.


SEQUENCE LISTING (34 sequences)

SEQ ID NO: 1 - protein, 166 aa, homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 2 - protein, 166 aa, homo sapiens
MSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 3 - DNA, 1240 bp, homo sapiens
cacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacaaaaaaaaaaaaaaaaaaaaaaaaaaaaaa

SEQ ID NO: 4 - protein, 166 aa, homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 5 - DNA, 876 bp, homo sapiens
agaacctagagcccaaggttcagagtcacccatctcagcaagcccagaagtatctgcaatatctacgatggcctcgccctttgctttactgatggtcctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataacatctggtccaacatgaaaacaattcttattgactcatacaccaggtcacgctttcatgaattctgtcatttcaaagactctcacccctgctataactatgaccatgctgataaactgatttatctatttaaatatttatttaactattcataagatttaaattatttttgttcatataacgtcatgtgcacctttacactgtggttagtgtaataaaacatgttccttatatttactc

SEQ ID NO: 6 - protein, 189 aa, homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 7 - DNA, 1142 bp, homo sapiens
gagaacctggagcctaaggtttaggctcacccatttcaaccagtctagcagcatctgcaacatctacaatggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgaaaactggttcaacatggaaatgattttcattgattcgtatgccagctcacctttttatgatctgccatttcaaagactcatgtttctgctatgaccatgacacgatttaaatcttttcaaatgtttttaggagtattaatcaacattgtattcagctcttaaggcactagtcccttacagaggaccatgctgactgatccattatctatttaaatatttttaaaatattatttatttaactatttataaaacaacttatttttgttcatattatgtcatgtgcacctttgcacagtggttaatgtaataaaatgtgttctttgtatttggtaaatttattttgtgttgttcattgaacttttgctatggaacttttgtacttgtttattctttaaaatgaaattccaagcctaattgtgcaacctgattacagaataactggtacacttcatttgtccatcaatattatattcaagatataagtaaaaataaactttctgtaaacca

SEQ ID NO: 8 - protein, 188 aa, homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 9 - DNA, 977 bp, homo sapiens
acaactagggaatttagaaaatggaaattagtatgttcactatttaagacctatgcacagagcaaagtcttcagaaaacctagaggccgaagttcaaggttatccatctcaagtagcctagcaatatttgcaacatcccaatggccctgtccttttctttactgatggccgtgctggtgctcagctacaaatccatctgttctctgggctgtgatctgcctcagacccacagcctgggtaataggagggccttgatactcctggcacaaatgggaagaatctctcatttctcctgcctgaaggacagacatgatttcggattccccgaggaggagtttgatggccaccagttccagaaggctcaagccatctctgtcctccatgagatgatccagcagaccttcaatctcttcagcacagaggactcatctgctgcttgggaacagagcctcctagaaaaattttccactgaactttaccagcaactgaatgacctggaagcatgtgtgatacaggaggttggggtggaagagactcccctgatgaatgaggactccatcctggctgtgaggaaatacttccaaagaatcactctttatctaacagagaagaaatacagcccttgtgcctgggaggttgtcagagcagaaatcatgagatccctctcgttttcaacaaacttgcaaaaaagattaaggaggaaggattgaaacctggttcaacatggaaatgatcctgattgactaatacattatctcacactttcatgagttcttccatttcaaagactcacttctataaccaccacgagttgaatcaaaattttcaaatgttttcagcagtgtgaagaagcttggtgtataccttgcaggcactagtcctttacagatgacaatgctgatgtctctgttcatctatttatttaaatatttatttatttttaaaatttaaattattttttatgtgatatcatgagt

SEQ ID NO: 10 - protein, 189 aa, homo sapiens
MALSFSLLMAVLVLSYKSICSLGCDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 11 - DNA, 570 bp, homo sapiens
atggccttgccctttgttttactgatggccctggtggtgctcaactgcaagtcaatctgttctctgggctgtgatctgcctcagacccacagcctgagtaacaggaggactttgatgataatggcacaaatgggaagaatctctcctttctcctgcctgaaggacagacatgactttggatttcctcaggaggagtttgatggcaaccagttccagaaggctcaagccatctctgtcctccatgagatgatccagcagaccttcaatctcttcagcacaaaggactcatctgctacttgggatgagacacttctagacaaattctacactgaactttaccagcagctgaatgacctggaagcctgtatgatgcaggaggttggagtggaagacactcctctgatgaatgtggactctatcctgactgtgagaaaatactttcaaagaatcaccctctatctgacagagaagaaatacagcccttgtgcatgggaggttgtcagagcagaaatcatgagatccttctctttatcagcaaacttgcaagaaagattaaggaggaaggaatga

SEQ ID NO: 12 - protein, 189 aa, homo sapiens
MALPFVLLMALVVLNCKSICSLGCDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 13 - DNA, 570 bp, homo sapiens
atggctttgccttttgctttactgatggccctggtggtgctcagctgcaagtcaagctgctctctggactgtgatctgcctcagacccacagcctgggtcacaggaggaccatgatgctcctggcacaaatgaggagaatctctcttttctcctgtctgaaggacagacatgacttcagatttccccaggaggagtttgatggcaaccagttccagaaggctgaagccatctctgtcctccatgaggtgattcagcagaccttcaatctcttcagcacaaaggactcatctgttgcttgggatgagaggcttctagacaaactctatactgaactttaccagcagctgaatgacctggaagcctgtgtgatgcaggaggtgtgggtgggagggactcccctgatgaatgaggactccatcctggctgtgagaaaatacttccaaagaatcactctctacctgacagagaaaaagtacagcccttgtgcctgggaggttgtcagagcagaaatcatgagatccttctcttcatcaagaaacttgcaagaaaggttaaggaggaaggaataa

SEQ ID NO: 14 - protein, 189 aa, homo sapiens
MALPFALLMALVVLSCKSSCSLDCDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 15 - DNA, 737 bp, homo sapiens
tacccacctcaggtagcctagtgatatttgcaaaatcccaatggcccggtccttttctttactgatggccgtgctggtactcagctacaaatccatctgctctctgggctgtgatctgcctcagacccacagcctgcgtaataggagggccttgatactcctggcacaaatgggaagaatctctcctttctcctgcttgaaggacagacatgaattcagattcccggaggaggagtttgatggccaccagttccagaagactcaagccatctctgtcctccatgagatgatccagcagaccttcaatctcttcagcacagaggactcatctgctgcttgggaacagagcctcctagaaaaattttccactgaactttaccagcaactgaatgacctggaagcatgtgtgatacaggaggttggggtggaagagactcccctgatgaatgaggacttcatcctggctgtgaggaaatacttccaaagaatcactctttatctaacagagaagaaatacagcccttgtgcctgggaggttgtcagagcagaaatcatgagatccttctctttttcaacaaacttgaaaaaaggattaaggaggaaggattgaaaactggttcatcatggaaatgattctcattgactaatgcatcatctcacactttcatgagttcttccatttcaaagactcacttctataaccaccacaagttgaatcaaaatttccaaatgttttc

SEQ ID NO: 16 - protein, 189 aa, homo sapiens
MARSFSLLMAVLVLSYKSICSLGCDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLTEKKYSPCAWEVVRAEIMRSFSFSTNLKKGLRRKD

SEQ ID NO: 17 - DNA, 1238 bp, homo sapiens
gtcatccatctgaaccagctcagcagcatccacaacatcctacaatggccttgactttttatttactggtcgccctagtggtgctcagctacaagtcattcagctctctgggctgtgatctgcctcagactcacagcctgggtaacaggagggccttgatactcctggcacaaatgcgaagaatctctcctttctcctgcctgaaggacagacatgactttgaattcccccaggaggagtttgatgataaacagttccagaaggctcaagccatctctgtcctccatgagatgatccagcagaccttcaacctcttcagcacaaaggactcatctgctgctttggatgagacccttctagatgaattctacatcgaacttgaccagcagctgaatgacctggagtcctgtgtgatgcaggaagtgggggtgatagagtctcccctgatgtacgaggactccatcctggctgtgaggaaatacttccaaagaatcactctatatctgacagagaagaaatacagctcttgtgcctgggaggttgtcagagcagaaatcatgagatccttctctttatcaatcaacttgcaaaaaagattgaagagtaaggaatgagacctggtacaacacggaaatgattcttatagactaatacagcagctcacacttcgacaagttgtgctctttcaaagacccttgtttctgccaaaaccatgctatgaattgaatcaaatgtgtcaagtgttttcaggagtgttaagcaacatcctgttcagctgtatgggcactagtcccttacagatgaccatgctgatggatctattcatctatttatttaaatctttatttagttaactatctatagggcttaaattagttttgttcatattatattatgtgaacttttatattgtgaattgtgtaacaaaaacatgttctttatatttattattttgccttgtttattaaatttttactatagaaaaattctttatttattctttaaaattgaactccaaccctgattgtgcaaactgattaaaggaagtggtgcacttgcaaacaagctctactatccctgaggaaataccagagactctggaaggtgatattcaaaaagcaaaaagcaaaattctaacactaattgaacctgacattaaaacagcacagatgactgctaccatagattcctgcctttcaaacgcagggcaagacattcattggtcatacgtagaaggc

SEQ ID NO: 18 - protein, 189 aa, homo sapiens
MALTFYLLVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLESCVMQEVGVIESPLMYEDSILAVRKYFQRITLYLTEKKYSSCAWEVVRAEIMRSFSLSINLQKRLKSKE

SEQ ID NO: 19 - DNA, 963 bp, homo sapiens
caaggttatccatctcaagtagcctagcaatatttgcaacatcccaatggccctgtccttttctttacttatggccgtgctggtgctcagctacaaatccatctgttctctgggctgtgatctgcctcagacccacagcctcggtaataggagggccttgatactcctgggacaaatgggaagaatctctcctttctcctgcctgaaggacagacatgatttccgaatcccccaggaggagtttgatggcaaccagttccagaaggctcaagccatctctgtcctccatgagatgatccagcagaccttcaatctcttcagcacagaggactcatctgctgcttgggaacagagcctcctagaaaaattttccactgaactttaccagcaactgaatgacctggaagcatgtgtgatacaggaggttggggtggaagagactcccctgatgaatgaggactccatcctggctgtgaggaaatacttccaaagaatcactctttatctaatagagaggaaatacagcccttgtgcctgggaggttgtcagagcagaaatcatgagatccctctcgttttcaacaaacttgcaaaaaagattaaggaggaaggattgaaaactggttcaacatggcaatgatcctgattgactaatacattatctcacactttcatgagttcttccatttcaaagactcacttctataaccacgacgcgttgaatcaaaattttcaaatgttttcagcagtgtaaagaagtgtcgtgtatacctgtgcaggcactagtcctttacagatgaccattctgatgtctctgttcatcttttgtttaaatatttatttaattatttttaaaatttatgtaatatcatgagtcgctttacattgtggttaatgtaacaatatatgttcttcatatttagccaatatattaatttcctttttcattaaatttttactatac

SEQ ID NO: 20 - protein, 189 aa, homo sapiens
MALSFSLLMAVLVLSYKSICSLGCDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 21 - DNA, 653 bp, homo sapiens
agagaacctagagcccaaggttcagagtcacccatctcagcaagcccagaagcatctgcaatatctatgatggcctcgccctttgctttactgatggccctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataacacctggtccaaca

SEQ ID NO: 22 - protein, 189 aa, homo sapiens
MASPFALLMALVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 23 - DNA, 985 bp, homo sapiens
ccaaggttcagtgttacccctcatcaaccagcccagcagcatcttcgggattcccaatggcattgccctttgctttaatgatggccctggtggtgctcagctgcaagtcaagctgctctctgggctgtaatctgtctcaaacccacagcctgaataacaggaggactttgatgctcatggcacaaatgaggagaatctctcctttctcctgcctgaaggacagacatgactttgaatttccccaggaggaatttgatggcaaccagttccagaaagctcaagccatctctgtcctccatgagatgatgcagcagaccttcaatctcttcagcacaaagaactcatctgctgcttgggatgagaccctcctagaaaaattctacattgaacttttccagcaaatgaatgacctggaagcctgtgtgatacaggaggttggggtggaagagactcccctgatgaatgaggactccatcctggctgtgaagaaatacttccaaagaatcactctttatctgatggagaagaaatacagcccttgtgcctgggaggttgtcagagcagaaatcatgagatccttctctttttcaacaaacttgcaaaaaagattaaggaggaaggattgaaaactggttcatcatggaaatgattctcattgactaatacatcatctcacactttcatgttcttccatttcaaagactcacttctataaccaccacaagttgaatcaaaatttccaaatgttttcaggagtgttaagaagcatcgtgtttacctgtgcaggcactagtcctttacagatgaccattctgatgtctcctttcatctatttatttaaatatttatttatttaactatttttattatttaaattattttttatgtaatatcatgagtacctttacattgtggttaatgtaacaaatatgttcttcatatttagccaatatattaatttcctttttcattaaatttttactat

SEQ ID NO: 24 - protein, 189 aa, homo sapiens
MALPFALMMALVVLSCKSSCSLGCNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSFSFSTNLQKRLRRKD

SEQ ID NO: 25 - DNA, 941 bp, homo sapiens
atcccaatggccctgtccttttctttactgatggccgtgctggtgctcagctacaaatccatctgttctctgggctgtgatctgcctcagactcacagcctgggtaataggagggccttgatactcctggcacaaatgggaagaatctctcatttctcctgcctgaaggacagatatgatttcggattcccccaggaggtgtttgatggcaaccagttccagaaggctcaagccatctctgccttccatgagatgatccagcagaccttcaatctcttcagcacaaaggattcatctgctgcttgggatgagaccctcctagacaaattctacattgaacttttccagcaactgaatgacctagaagcctgtgtgacacaggaggttggggtggaagagattgccctgatgaatgaggactccatcctggctgtgaggaaatactttcaaagaatcactctttatctgatggggaagaaatacagcccttgtgcctgggaggttgtcagagcagaaatcatgagatccttctctttttcaacaaacttgcaaaaaggattaagaaggaaggattgaaaactcattcaacatggaaatgatcctcattgattaatacatcatctcacactttcatgagttcttccatttcaaagactcacttctataaccaccacaagttgaatcaaaatttcaaaatgttttcaggagtgtaaagaagcatcgtgtttacctgtgcaggcactagtcctttacagatgaccatgctgatgtctctattcatctatttatttaaatatttatttatttaactatttttaaggtttaaatcatgttttatgtaatatcatgtgtacctttacattttgcttaatgtaacaatatatgttcttcatatttagttaatatattaacttccttttcattaaatttttactatacgc

SEQ ID NO: 26 - protein, 189 aa, homo sapiens
MALSFSLLMAVLVLSYKSICSLGCDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKD

SEQ ID NO: 27 - DNA, 980 bp, homo sapiens
gttcaaggttacccatctcaagtagcctagcaacatttgcaacatcccaatggccctgtccttttctttactgatggccgtgctggtgctcagctacaaatccatctgttctctaggctgtgatctgcctcagacccacagcctgggtaataggagggccttgatactcctggcacaaatgggaagaatctctcctttctcctgcctgaaggacagacatgactttggacttccccaggaggagtttgatggcaaccagttccagaagactcaagccatctctgtcctccatgagatgatccagcagaccttcaatctcttcagcacagaggactcatctgctgcttgggaacagagcctcctagaaaaattttccactgaactttaccagcaactgaataacctggaagcatgtgtgatacaggaggttgggatggaagagactcccctgatgaatgaggactccatcctggctgtgaggaaatacttccaaagaatcactctttatctaacagagaagaaatacagcccttgtgcctgggaggttgtcagagcagaaatcatgagatccctctctttttcaacaaacttgcaaaaaatattaaggaggaaggattgaaaactggttcaacatggcaatgatcctgattgactaatacattatctcacactttcatgagttcctcaatttcaaagactcacttctataaccaccacgagttgaatcaaaattttcaaatgttttcagcagtgtaaagaagcgtcgtgtatacctgtgcaggcactagtactttacagatgaccatgctgatgtctctgttcatctatttatttaaatatttatttaattatttttaagatttaaattatttttttatgtaatatcatgtgtacctttacattgtggtgaatgtaacaatatatgttcttcatatttagccaatatattaatttcctttttcattaaatttttactatac

SEQ ID NO: 28 - protein, 189 aa, homo sapiens
MALSFSLLMAVLVLSYKSICSLGCDLPQTHSLGNRRALILLAQMGRISPFSCLKDRHDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 29 - DNA, 985 bp, homo sapiens
acatcccaatggccctgtccttttctttactgatggccgtgctggtgctcagctacaaatccatctgttctctgggctgtgatctgcctcagacccacagcctgggtaataggagggccttgatactcctggcacaaatgggaagaatctctcctttctcctgcctgaaggacagacatgactttggattcccccaagaggagtttgatggcaaccagttccagaaggctcaagccatctctgtcctccatgagatgatccagcagaccttcaatctcttcagcacaaaggactcatctgctacttgggaacagagcctcctagaaaaattttccactgaacttaaccagcagctgaatgacatggaagcctgcgtgatacaggaggttggggtggaagagactcccctgatgaatgtggactccatcttggctgtgaagaaatacttccaaagaatcactctttatctgacagagaagaaatacagcccttgtgcttgggaggttgtcagagcagaaatcatgagatccttctctttatcaaaaatttttcaagaaagattaaggaggaaggaatgaaaccgtttcaacatggaaatgatctgtattgactaatacaccagtccacacttctatgacttctgccatttcaaagactcatttctcctataaccaccgcatgagttgaatcaaaattttcagatcttttcaggagtgtaaggaaacatcatgtttacctgtgcaggcactagtcctttacagatgaccatgctgatagatctaattatctatctattgaaatatttatttatttattagatttaaattatttttgtccatgtaatattatgtgtacttttacattgtgttatatcaaaatatgttaatctaatatttagtcaatatattattttctttttattaatttttactattaaaacttcttatattatttggttattctttaataaagaaattccaagccc

SEQ ID NO: 30 - protein, 189 aa, homo sapiens
MALSFSLLMAVLVLSYKSICSLGCDLPQTHSLGNRRALILLAQMGRISPFSCLKDRHDFGFPQEEFDGNQFQKAQAISVLHEMIQQTFNLFSTKDSSATWEQSLLEKFSTELNQQLNDMEACVIQEVGVEETPLMNVDSILAVKKYFQRITLYLTEKKYSPCAWEVVRAEIMRSFSLSKIFQERLRRKE

SEQ ID NO: 31 - DNA, 573 bp, muscus muscus
atggctagactctgtgctttcctcgtgatgctgatagtgatgagctactggtcaatctgttctctaggatgcgatctgcctcacacttataacctcaggaacaagagggccttgaaggtcctggcacagatgaggaggctcccctttctctcctgcctgaaggacaggcaggactttggattccccctggagaaggtggataaccagcagatccagaaggctcaagccatccctgtgctgcgagatcttactcagcagaccttgaacctcttcacatcaaaggcttcatctgctgcttggaatgcaaccctcctagactcattctgcaatgacctccaccagcagctcaatgacctgcaaacctgtctgatgcagcaggtgggggtgcaggaacctcctctgacccaggaagacgccctgctggctgtgaggaaatatttccacaggatcactgtgtacctgagagagaagaaacacagcccctgtgcctgggaggtggtcagagcagaagtctggagagccctgtcttcctcagtcaacttgctgccaagactgagtgaagagaaggagtga

SEQ ID NO: 32 - protein, 190 aa, muscus muscus
MARLCAFLVMLIVMSYWSICSLGCDLPHTYNLRNKRALKVLAQMRRLPFLSCLKDRQDFGFPLEKVDNQQIQKAQAIPVLRDLTQQTLNLFTSKASSAAWNATLLDSFCNDLHQQLNDLQTCLMQQVGVQEPPLTQEDALLAVRKYFHRITVYLREKKHSPCAWEVVRAEVWRALSSSVNLLPRLSEEKE

SEQ ID NO: 33 - DNA, 549 bp, muscus muscus
atgaacaacaggtggatcctccacgctgcgttcctgctgtgcttctccaccacagccctctccatcaactataagcagctccagctccaagaaaggacgaacattcggaaatgtcaggagctcctggagcagctgaatggaaagatcaacctcacctacagggcggacttcaagatccctatggagatgacggagaagatgcagaagagttacactgcctttgccatccaagagatgctccagaatgtctttcttgtcttcagaaacaatttctccagcactgggtggaatgagactattgttgtacgtctcctggatgaactccaccagcagacagtgtttctgaagacagtactagaggaaaagcaagaggaaagattgacgtgggagatgtcctcaactgctctccacttgaagagctattactggagggtgcaaaggtaccttaaactcatgaagtacaacagctacgcctggatggtggtccgagcagagatcttcaggaactttctcatcattcgaagacttaccagaaacttccaaaactga

SEQ ID NO: 34 - protein, 182 aa, muscus muscus
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN